(19) United States
(12) Patent Application Publication
(10) Pub. No.: US 2025/0282872 A1
(43) Pub. Date: Sep. 11, 2025

(54) B AND T LYMPHOCYTE ATTENUATOR (BTLA) MODULATORS AND METHOD OF USING SAME

(71) Applicant: AnaptysBio, Inc., San Diego, CA (US)

(72) Inventors: Martin Edward Dahl, San Diego, CA (US); Stephen Parmley, San Diego, CA (US); Paul Lizzul, San Diego, CA (US); Eric Hare, San Diego, CA (US)

(21) Appl. No.: 18/859,958

(22) PCT Filed: Apr. 26, 2023
(86) PCT No.: PCT/US2023/066258

§ 371 Date: Oct. 24, 2024

Related U.S. Application Data
(60) Provisional application No. 63/335,623, filed on Apr. 27, 2022.

(51) Int. Cl. C07K 16/28; A61K 39/00; A61P 37/06

(52) U.S. Cl. CPC C07K 16/2818; A61P 37/06; A61K 2039/505; A61K 2039/54; A61K 2039/545; C07K 2317/24; C07K 2317/34; C07K 2317/75; C07K 2317/76; C07K 2317/92

(57) ABSTRACT

A BTLA-binding agent and immunoglobulin heavy chain and light chain polypeptides of the binding agent, as well as methods of using the BTLA-binding agent to treat a disorder or disease that is responsive to BTLA agonism, such as an autoimmune or inflammatory disease.

### CROSS-REFERENCE TO RELATED APPLICATIONS

This patent application claims priority to U.S. provisional patent application 63/335,623, filed on Apr. 27, 2022, the entire disclosure of which is hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 339,641 Byte XML file named “767170.XML” dated Apr. 12, 2023.

### BACKGROUND OF THE INVENTION

T lymphocytes are activated from a naïve state by a combination of T cell receptor (TCR) engagement and positive signals coming from costimulatory molecules. Conversely, coinhibitory molecules play important roles in modulating T cell activity by delivering negative signals to counter balance positive costimulatory signals. Coinhibitory molecules act as checkpoints to maintain immunologic tolerance to self and to control activated T cells after resolving immune insults such as infections and inflammation.

CD28 is the dominant costimulatory molecule on T cells that, once engaged with the ligands B7.1 or B7.2 on the surface of antigen-presenting cells (APC), delivers intracellular signals that enhance T cell proliferation and differentiation with concomitant TCR engagement. T cell proliferation and effector functions are inhibited when the same ligands engage the coinhibitory molecule cytotoxic T lymphocyte antigen-4 (CTLA-4) on T cells (Chambers et al., Ann. Rev. Immunol., 19:565-594, 2001; Egen et al., Nature Immunol., 3:611-618, 2002). Similarly, T cell proliferation and effector functions are inhibited when cells expressing PD-L1 engage the coinhibitory molecule PD-1 on T cells (Carter et al. Eur J Immunol., 32:634-43, 2002).

Dysfunction in negative checkpoint signals can contribute to chronic inflammatory conditions by preventing the suppressive signals that normally control autoreactive B and T lymphocytes. In patients with autoimmune disorders, the immune system reacts to normal body tissues as if they were foreign and tissues can become infiltrated with activated T cells and B cells that have broken tolerance to self-antigens. As a consequence, the autoimmune T and B lymphocytes mediate inflammation and tissue damage. Lymphocytes expressing coinhibitory molecules such as B and T lymphocyte attenuator (BTLA), cytotoxic T lymphocyte antigen-4 (CTLA-4), and PD-1 are normally suppressed by other immune or non-immune cells in the tissues that express the corresponding ligands.

Knock-out of coinhibitory molecules or pharmacologic blockade of the coinhibitory interactions has been shown to release the suppressive breaks and induce expansion of tumor-specific T cell populations and direct them to attack and kill tumor cells in animal models of various cancers. Mice with BTLA knock-out have increased sensitivity to experimental autoimmune encephalomyelitis (Watanabe et al., Nat. Immunol., 4:670-679, 2003). Conversely, agonist antibodies targeting mouse BTLA have been shown to suppress T cell activity and have demonstrated efficacy in mouse models of graft versus-host disease (GvHD) (Albring et al., J. Exp. Med., 207:2551-2559, 2010).

The ligand for BTLA is the Herpes virus entry mediator (HVEM), also known as tumor necrosis factor receptor superfamily member 14 (TNFRSF14). HVEM is also a positive costimulatory molecule that binds two secreted growth factors, lymphotoxin alpha and LIGHT, and is also used by herpes simplex virus (HSV) for entry into cells. Engagement of BTLA on T cells by HVEM expressed on tumor cells or other immune cells results in negative, suppressive signals. Anti-BTLA antibodies that bind and agonize BTLA could induce direct negative signals similar to that delivered by the native ligand HVEM thereby suppressing autoreactive T cell and B cells responses in autoimmune and inflammatory diseases. Furthermore, agonist antibodies that engage BTLA without interfering with the natural HVEM-BTLA interaction could enhance the natural coinhibitory signal.

Thus, agents capable of binding BTLA and modulating the immune checkpoint signals are needed.

### BRIEF SUMMARY OF THE INVENTION

Provided herein are BTLA-binding agents comprising immunoglobulin heavy and light chains polypeptides for use in a method of modulating BTLA signaling, suppressing an immune response, or treating an autoimmune or inflammatory disease.

Related compositions and methods also are provided, as will be apparent from the following detailed description.

### BRIEF DESCRIPTION OF THE SEVERAL VIEWS OF THE DRAWING(S)

FIG. 1 depicts the results of surface plasmon resonance binding kinetics of the 6G3 antibody to human BTLA and cynomolgus monkey BTLA extracellular domain.

FIG. 2 is a graph depicting the results of Kinetic Exclusion Assay binding kinetics of the 6G3 antibody to human BTLA and cynomolgus monkey BTLA extracellular domain.

FIG. 3 is a graph depicting the results of binding of the 6G3 antibody to 293c18 cells stably transfected with human BTLA and cynomolgus monkey BTLA.

FIG. 4 is a graph depicting the results of binding of the 6G3 antibody to normal donor human peripheral blood CD4⁺ T cells, CD8⁺ T cells, and CD20⁺ B cells.

FIG. 5A is a graph depicting the results of binding of the 6G3 antibody to normal cynomolgus monkey peripheral blood CD3⁺ T cells.

FIG. 5B is a graph depicting the results of binding of the 6G3 antibody to normal cynomolgus monkey peripheral blood CD20⁺ B cells.

FIG. 5C is a flow cytometry dot plot depicting the results of binding of a reference anti-BTLA antibody to normal cynomolgus monkey peripheral blood CD3⁻ and CD3⁻ cells.

FIG. 5D is a flow cytometry dot plot depicting the results of binding of the 6G3 antibody to normal cynomolgus monkey peripheral blood CD3⁺ and CD3⁻ cells.

FIG. 6 is a graph depicting the results of a competition assay, which illustrates the ability of anti-BTLA antibodies to compete with HVEM-Fc and a pre-formed HVEM-Fc/trimeric LIGHT complex for binding to 293c18 cells stably transfected with human BTLA.

FIG. 7A is a ribbon-model illustration of the crystal structure of human BTLA extracellular domain (black) docked with a space-filling model of the crystal structure of human HVEM extracellular binding domain (gray). The model depicts the results of a hydrogen-deuterium exchange experiment, which maps the peptides on human BTLA bound by the 6G3 antibody.

FIG. 7B is a ribbon-model illustration of the crystal structure of human BTLA extracellular domain (black) docked with a space-filling model of the crystal structure of human HVEM extracellular binding domain (gray). The molecule is rotated by 30° as compared to the view of the molecule shown in FIG. 7A and depicts the results of a hydrogen-deuterium exchange experiment, which maps the peptides on human BTLA bound by a reference anti-BTLA antagonist antibody.

FIG. 8 is a graph depicting the inhibitory activity of the 6G3 antibody in an HVEM-NF-κB HEK 293 luciferase reporter assay measuring LIGHT-induced HVEM signaling when BTLA and HVEM are expressed on the same cell.

FIG. 9A is a graph depicting the results of a fluorescence resonance energy transfer assay measuring association of BTLA and HVEM on the surface of transfected 293c18 cells, which illustrates the ability of anti-BTLA antibodies to compete with BTLA and HVEM binding on the same cell surface.

FIG. 9B is a graph depicting the results of a fluorescence resonance energy transfer assay measuring association of BTLA and HVEM on the surface of transfected 293c18 cells, which illustrates the ability of fluorescence donor anti-BTLA antibodies to generate an energy transfer signal with an anti-HVEM acceptor antibody.

FIG. 10 is a graph depicting the partial inhibitory activity of the 6G3 antibody in an HVEM-NF-κB HEK 293 luciferase reporter assay measuring BTLA-induced HVEM signaling when BTLA and HVEM are expressed on different cells.

FIG. 11 is a graph depicting the agonist activity of the 6G3 antibody added as a soluble antibody in an SHP2 recruitment PathHunter Jurkat BTLA signaling assay.

FIG. 12 is a graph depicting the inhibitory activity of anti-BTLA antibodies in an SHP2 recruitment PathHunter Jurkat BTLA signaling assay, where BTLA signaling was induced by HVEM on a transfected U-2 OS cell line.

FIG. 13 is a graph depicting the agonist activity of the 6G3 antibody in an SHP2 recruitment PathHunter Jurkat BTLA signaling assay with addition of FcγRIa transfected U-2 OS cells to provide FcγR engagement.

FIG. 14A is a schematic of the xenogeneic NSG/Hu-PBMC mouse model for the Graft vs. Host Disease study described herein, in accordance with embodiments of the invention.

FIG. 14B is a schematic showing the timeline, dosing schedule, and model groups of the NSG/Hu-PBMC Graft vs. Host Disease study described herein, in accordance with embodiments of the invention.

FIG. 14C is a graph depicting the results of overall survival in the NSG/Hu-PBMC Graft vs. Host Disease study for animal groups dosed twice weekly with either 1 mg/kg, 3 mg/kg, or 10 mg/kg of the 6G3 antibody.

FIG. 15 is a plot that shows individual and mean (SD) concentrations of human sBTLA per dose group in a humanized murine model of GvHD following dosing with 6G3 antibody at 1, 3, and 10 mg/kg (IP) twice/week. Plasma samples were collected at a mid-point in the study via cardiac bleed.

FIG. 16 is a plot that shows mean (SD) serum concentrations of cyno sBTLA per dose group in cynomolgus monkeys following dosing with 6G3 IgG4. All animals were administered a single dose of 6G3 IgG4 either IV or SC and blood samples were collected from all animals in all groups predose, 3, 6, 12, 24, 48, 72, 96, 168, 240, 336, 504, 672, and 840 hours postdose.

FIG. 17 is a plot that shows mean (SD) serum concentrations of cyno sBTLA per dose group in cynomolgus monkeys following dosing with 6G3 IgG4. All animals were administered a weekly doses of 6G3 IgG4 on Days 1, 8, and 15 either IV or SC and blood samples were collected from all animals in all groups on Days 1, 8, and 15: Predose, 3, 24, 48, 72, 96 hours postdose.

FIG. 18 is a plot that shows mean (SD) serum concentrations of 6G3 IgG4 (μg/ml) per dose group in cynomolgus monkeys following dosing with 6G3 IgG4. All animals were administered a weekly dose of 6G3 IgG4 on Days 1, 8, and 15 either IV or SC and blood samples were collected from all animals in all groups on Days 1, 8, and 15: Predose, 3, 24, 48, 72, 96 hours postdose.

FIG. 19 is four plots showing various data per dose group in cynomolgus monkeys following dosing with 6G3 IgG4, an isotype control, or CTLA-4-Ig control. The first plot shows BTLA Expression (MFI) per dose group. The second plot shows T cell percentage BTLA+per dose group. The third plot shows the number of human T cells per μl blood per dose group. The fourth plot shows percent CD25 positive per dose group.

FIG. 20 is four plots showing receptor occupancy and BTLA surface expression of T cells and B cells per dose group in cynomolgus monkeys following dosing with 6G3 IgG4. All animals were administered a weekly dose of 6G3 IgG4 or a control on Days1, 8, and 15 either IV or SC. The first plot shows the percent change from baseline of free receptors on T cells. The second plot shows the percent change from baseline of BTLA expression on T cells. The third plot shows the percent change from baseline of free receptors on B cells. The fourth plot shows the percent change from baseline of BTLA expression on B cells.

FIG. 21A is two histograms of healthy control and atopic dermatitis donors presented as the overlaid histograms of isotype control onto 6G3 IgG4 treated CD3⁺ T-cells.

FIG. 21B is two plots showing reduction in T cell proliferation by 6G3 IgG4 in healthy controls and atopic dermatitis donors shown as percentage reduction in proliferation (left) and division index (right).

FIG. 21C is two plots showing IFNγ levels of healthy control and atopic dermatitis donors PBMC culture supernatant 72 hours after anti-CD3 and anti-CD28 stimulation in the presence or absence of 100 nM of 6G3 IgG4 or isotype control.

FIG. 21D is a plot showing the surface BTLA expression level (plotted as mean fluorescence intensity (MFI)) on CD3+ T-cells from healthy controls and atopic dermatitis donors.

FIG. 22A is a plot of receptor occupancy on CD3+ cells following 400 mg SC dosing. The data shows that the 400 mg SC dose provides a half-life of about 9 days and sufficient pharmacokinetic exposure for full receptor occupancy (˜7 μg/mL) lasting greater than 30 days.

FIG. 22B is a plot of receptor occupancy on CD19⁺ cells following 400 mg SC dosing. The data shows that the 400 mg SC dose provides a half-life of about 9 days and sufficient pharmacokinetic exposure for full receptor occupancy (˜7 μg/mL) lasting greater than 30 days.

FIG. 23A is a plot of percent mice remaining alive vs. study day following 10 mg/kg single dosing with 6G3 antibody, 3 mg/kg biweekly dosing for 4 weeks with 6G3 antibody, 10 mg/kg single dosing with an IgG4 control antibody, 3 mg/kg biweekly dosing for 4 weeks with an IgG4 control antibody, or untreated animals in a GvHD model.

FIG. 23B is a plot of weight (% from start) of mice vs. study day following 10 mg/kg single dosing with 6G3 antibody, 3 mg/kg biweekly dosing for 4 weeks with 6G3 antibody, 10 mg/kg single dosing with an IgG4 control antibody, 3 mg/kg bi weekly dosing for 4 weeks with an IgG4 control antibody, or untreated animals in a GvHD model.

### DETAILED DESCRIPTION OF THE INVENTION

Provided herein is a BTLA-binding agent comprising immunoglobulin heavy chain and light chain polypeptides. BTLA is a 30 kilodalton (kD) type 1 transmembrane protein with an immunoglobulin-like extracellular domain, an immunoreceptor tyrosine-based inhibitory motif (ITIM), and an immunoreceptor tyrosine-based switch motif (ITSM). BTLA is expressed on B cells and T cells and acts a negative regulator of both B and T cell activity through interaction with its receptor, Herpes virus entry mediator (HVEM), expressed on tumor cells or APCs (Watanabe et al., Nat. Immunol., 4:670-679, 2003). In some embodiments, the BTLA-binding agent binds to BTLA without inhibiting binding between BTLA and HVEM. In one aspect, the BTLA-binding agent enhances binding between BTLA and HVEM.

The PD-1 binding agent comprises an immunoglobulin heavy chain variable region and an immunoglobulin light chain variable region, each of which comprise three complementarity determining regions (CDRs), usually referred to as CDR1, CDR2, or CDR3. The CDR regions also can be referred to using an “H” or “L” in the nomenclature to denote the heavy or light chain, respectively, i.e., CDRH1, CDRH2, CDRH3, CDRL1, CDRL2, or CDRL3. The CDRs of a given Ig sequence can be determined by any of several conventional numbering schemes, such as Kabat, Chothia, Martin (Enhanced Chothia), IGMT, or AHo (these are commonly used names for numbering schemes widely known in the field and described in published literature see, e.g., Kabat, et al., Sequences of Proteins of Immunological Interest, U.S. Department of Health and Human Services, NIH (1991) describing the “Kabat” numbering scheme; Chothia, et al., Canonical Structures for the Hypervariable Regions of Immunoglobulins, J. Mol. Biol., 196:901-917 (1987) and Al-Lazikani et al., Standard Conformations for the Canonical Structures of Immunoglobulins, J. Mol. Biol., 273:927-948 (1997) describing the “Chothia” numbering scheme; Abhinandan et al., Analysis and Improvements to Kabat and Structurally Correct Numbering of Antibody Variable Domains, Mol. Immunol., 45:3832-3839 (2008) describing the “Martin” or “Enhanced Chothia” numbering scheme; Lefranc et al., The IMGT unique numbering for immunoglobulins, T cell Receptors and Ig-like domains, The Immunologist, 7:132-136 (1999) and Lefranc et al., IMGT unique numbering for immunoglobulin and T cell receptor variable domains and I superfamily V-like domains, Dev. Comp. Immunol., 27:55-77 (2003) describing the “IMGT” numbering scheme; and Honegger et al., Yet another numbering scheme for immunoglobulin variable domains: an automatic modeling and analysis tool, J. Mol. Biol. 309:657-670 (2001) describing the “AHo” numbering scheme). The BTLA-binding agents provided herein are man-made and non-naturally occurring. They have been generated by laboratory techniques and, thus, are properly considered recombinant or synthetic molecules comprising recombinant or synthetic amino acid sequences. The immunoglobulin heavy and light chain polypeptides can be “isolated” in the sense that they are removed from the environment in which they are produced (e.g., cell culture) and purified to any degree.

According to one aspect of the disclosure, the BTLA-binding agent comprises immunoglobulin heavy chain polypeptide of the BTLA-binding agent comprises the amino acid sequence of any one of SEQ ID NOs: 1-15, 207, 208, 217, or 218, or at least the CDRs thereof; or comprises an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to any one of SEQ ID NOs 1-15, 207, 208, 217, or 218. The CDRs can be as determined using any known numbering scheme, such as such as Kabat, Chothia, Martin (Enhanced Chothia), IGMT, or AHo. In some embodiments, CDR1, CDR2, and CDR3 comprise residues 31-35 (CDRH1), 50-66 (CDRH2), and 99-106 (CDRH3) of SEQ ID NOs: 1-15, 207, 208, 217, or 218.

In some embodiments, the immunoglobulin heavy chain comprises the following CDRs:
(a) a CDRH1 comprising Asp Tyr Thr Ile His (SEQ ID NO: 27);
(b) a CDRH2 comprising Trp Ile Tyr Pro Gly Ser Gly Asn Thr Lys Tyr Asn Asp Xaa1 Phe Lys Xaa2 (SEQ ID NO: 30), wherein Xaa1 is lysine (Lys) or glutamic acid (Glu), and Xaa2 is aspartic acid (Asp) or valine (Val) (e.g., SEQ ID NO: 28, 30, 31, 212, or 222); and
(c) a CDRH3 comprising Arg Xaa1 Xaa2 Tyr Xaa3 Met Xaa4 Tyr (SEQ ID NO: 32), wherein Xaa1 is asparagine (Asn) or serine (Ser), Xaa2 is tyrosine (Tyr) or histidine (His), Xaa3 is alanine (Ala) or valine (Val), and Xaa4 is glutamic acid (Glu) or aspartic acid (Asp). Examples of such CDRH3 sequences include, for instance, SEQ ID NOs: 29, 33, 34, 213, or 223.

In some embodiments, the immunoglobulin heavy chain polypeptide comprises the amino acid sequence Gln Val Gln Leu Val Gln Ser Gly Ala Glu Val Lys Lys Pro Gly Ala Ser Val Lys Val Ser Cys Lys Ala Ser Gly Xaa1 Thr Xaa2 Thr Asp Tyr Thr Ile His Trp Val Arg Gln Ala Pro Gly Gln Arg Leu Glu Trp Met Gly Trp Ile Tyr Pro Gly Ser Gly Asn Thr Lys Tyr Asn Asp Xaa3 Phe Lys Xaa4 Arg Val Thr Ile Thr Xaa5 Asp Xaa6 Ser Xaa7 Xaa8 Thr Ala Tyr Met Glu Leu Ser Ser Leu Arg Ser Glu Asp Thr Ala Val Tyr Xaa9 Cys Ala Arg Arg Xaa10 Xaa11 Tyr Xaa12 Met Xaa13 Tyr Trp Gly Gln Gly Thr Thr Val Thr Val Ser Ser Ala (SEQ ID NO: 26), or at least the CDR regions thereof, wherein
Xaa1 is phenylalanine (Phe) or tyrosine (Tyr),
Xaa2 is phenylalanine (Phe) or leucine (Leu),
Xaa3 is lysine (Lys) or glutamic acid (Glu),
Xaa4 is aspartic acid (Asp) or valine (Val),
Xaa5 is alanine (Ala) or arginine (Arg),
Xaa6 is lysine (Lys) or threonine (Thr),
Xaa7 is alanine (Ala) or serine (Ser),
Xaa8 is serine (Ser) or threonine (Thr),
Xaa9 is tyrosine (Tyr) or phenylalanine (Phe), and
Xaa10 is asparagine (Asn) or serine (Ser),
Xaa11 is tyrosine (Tyr) or histidine (His),
Xaa12 is alanine (Ala) or valine (Val), and
Xaa13 is glutamic acid (Glu) or aspartic acid (Asp).
   In some embodiments, the Ig heavy chain polypeptide comprises SEQ ID NO: 26, provided that it retains the same CDRs (CDR1, CDR2, and CDR3) of any of SEQ ID NOs: 1-15, 207, 208, 217, or 218.

According to this aspect of the disclosure, the immunoglobulin light chain polypeptide of the BTLA-binding agent can comprise the amino acid sequence of any one of SEQ ID NOs: 16-25, 209, 210, 219, or 220, or at least the CDRs thereof; or an amino acid sequence with at least 80% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to any one of SEQ ID NOS: 16-25, 209, 210, 219, or 220. As with the Ig heavy chain, the CDRs can be as determined using any known numbering scheme, such as such as Kabat, Chothia, Martin (Enhanced Chothia), IGMT, or AHo. In some embodiments, CDR1, CDR2, and CDR3 comprise residues 24-34 (CDRL1), 50-56 (CDRL2), and 89-97 (CDRL3) of SEQ ID NOs: 16-25, 209, 210, 219, or 220.

In some embodiments, the Ig light chain comprises the following CDRs:
(a) a CDRL1 comprising Lys Ala Ser Gln Asn Val Phe Thr Asn Val Ala (SEQ ID NO: 36);
(b) a CDRL2 comprising Ser Ala Ser Tyr Arg Xaa Ser (SEQ ID NO: 39), wherein Xaa is tyrosine (Tyr) or serine (Ser) (e.g., SEQ ID NO: 37, 40, 215, or 225); and
(c) a CDRL3 comprising Gln Gln Tyr Xaa1 Xaa2 Tyr Pro Tyr Thr (SEQ ID NO: 41), wherein Xaa1 is serine (Ser) or asparagine (Asn), and Xaa2 is threonine (Thr) or serine (Ser) (e.g., SEQ ID NO: 38, 41, 42, 216, or 226).

In other embodiments, the immunoglobulin light chain polypeptide comprises the amino acid sequence Asp Ile Val Met Thr Gln Ser Pro Asp Ser Leu Ala Val Ser Leu Gly Glu Arg Ala Thr Ile Asn Cys Lys Ala Ser Gln Asn Val Phe Thr Asn Val Ala Trp Tyr Gln Gln Lys Pro Gly Gln Xaa1 Pro Lys Xaa2 Leu Ile Tyr Ser Ala Ser Tyr Arg Xaa3 Ser Gly Val Pro Asp Arg Phe Ser Gly Ser Gly Ser Gly Thr Asp Phe Thr Leu Thr Ile Ser Ser Leu Gln Ala Glu Asp Val Ala Val Tyr Xaa4 Cys Gln Gln Tyr Xaa5 Xaa6 Tyr Pro Tyr Thr Phe Gly Gln Gly Thr Lys Leu Glu Ile Lys Arg (SEQ ID NO: 35), or at least the CDR regions thereof, wherein
Xaa1 is serine (Ser) or proline (Pro),
Xaa2 is proline (Pro) or leucine (Leu),
Xaa3 is tyrosine (Tyr) or serine (Ser),
Xaa4 is tyrosine (Tyr) or phenylalanine (Phe),
Xaa5 is serine (Ser) or asparagine (Asn), and
Xaa6 is threonine (Thr) or serine (Ser).
   In some embodiments, the Ig light chain polypeptide comprises SEQ ID NO: 35, provided that it retains the same CDRs (CDR1, CDR2, and CDR3) of any one of SEQ ID NOs: 16-25, 209, 210, 219, or 220.

In another aspect of the disclosure, the BTLA-binding agent comprises an immunoglobulin heavy chain polypeptide comprising any one of SEQ ID NOs: 43-156, or at least the CDRs thereof; or an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to any one of SEQ ID NOs: 43-156. The CDRs can be as determined using any known numbering scheme, such as such as Kabat, Chothia, Martin (Enhanced Chothia), IGMT, or AHo. In some embodiments, CDR1, CDR2, and CDR3 comprise residues 31-35 (CDRH1), 50-66 (CDRH2), and 99-113 (CDRH3) of SEQ ID NOs: 43-156, with the exception that in SEQ ID NO: 66, CDRH1 can be residues 50-67 and CDRH3 can be residues 100-114; and in SEQ ID NOs: 141, 150, 152, 153, 155, and 156 CDRH3 can be residues 100-114.

In some embodiments, the immunoglobulin heavy chain polypeptide comprises the following CDRs:
(a) a CDRH1 comprising X¹SX²MN (SEQ ID NO: 195), wherein X¹ is N or T, and X² is W, F, H, G, P, R, K, D, S, L, V, N, or Y;
(b) a CDRH2 comprising RIYPX¹GX²X³DTNYX⁴GKFK (SEQ ID NO: 196), wherein:
   X¹ is absent or A;
   X² is D, Y, Q, G, L, F, H, S, P, R, or T;
   X³ is G, Y, A, F, S, D, V, T, E, K, or R; and
   X⁴ is N, V, Q, R, A, F, Y, S, G, P, or T; and
(c) a CDRH3 comprising X¹SGTFX²X³GNYX⁴X⁵YFDV (SEQ ID NO: 197), wherein:
   X¹ is K or R;
   X² is N or D;
   X³ is D, S, F, Y, F, V, S, G, T, R, I, L, or E;
   X⁴ is R or H; and
   X⁵ is W, R, F, L, N, Y, P, I, V, A, S, G, R, or K.

In some embodiments, the Ig heavy chain comprises a CDRH1 comprising SEQ ID NO: 201; a CDRH2 comprising SEQ ID NO: 202; and a CDRH3 comprising SEQ ID NO: 203.

In some embodiments, the BTLA-binding agent comprises an immunoglobulin heavy chain polypeptide comprising the sequence:

(SEQ ID NO: 193)
QVQLVQSGAEVKKPGSSVKVSCKASGYX₁FSX₂SX₃MNWVRQAPG
QGLEWMGRIYPX₄GX₅X₆DTNYX₇GKFKGRVTITADKX₈TX₉TAY
MELX₁₀SLRSEX₁₁TAVX₁₂YX₁₃CAX₁₄SGTFX₁₅X₁₆GNYX₁₇
X₁₈YFDVWGKGTTVTVSSA,

or at least the CDR regions thereof, wherein
X¹ is A or V;
X² is N or T;
X³ is W, F, H, G, P, R, K, D, S, L, V, N, or Y;
X⁴ is absent or A;
X⁵ is D, Y, Q, G, L, F, H, S, P, R, or T;
X⁶ is G, Y, A, F, S, D, V, T, E, K, or R;
X¹ is N, V, Q, R, A, F, Y, S, G, P, or T;
X⁸ is S or F;
X⁹ is S, T, or N;
X¹⁰ is S or R;
X¹¹ is D or V;
X¹² is absent or Y;
X¹³ is Y or F;
X¹⁴ is K or R;
X¹⁵ is N or D;
X¹⁶ is D, S, F, Y, F, V, S, G, T, R, I, L, or E;
X¹⁷ is R or H; and
X¹⁸ is W, R, F, L, N, Y, P, I, V, A, S, G, R, or K.
   In some embodiments, the Ig heavy chain polypeptide comprises SEQ ID NO: 193, provided that it retains the same CDRs (CDR1, CDR2, and CDR3) of any of SEQ ID NOs: 43-156.

According to this aspect of the disclosure, the binding agent further comprises an Ig light chain comprising any of SEQ ID NOs: 157-192, or at least the CDRs thereof; or an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to any one of SEQ ID NOs: 157-192. The CDRs can be as determined using any known numbering scheme, such as such as Kabat, Chothia, Martin (Enhanced Chothia), IGMT, or AHo. In some embodiments, CDR1, CDR2, and CDR3 comprise residues 24-34 (CDRL1), 50-56 (CDRL2), and 89-97 (CDRL3) of SEQ ID NOs: 157-192.

In some embodiments, the BTLA-binding agent can comprise an immunoglobulin light chain polypeptide comprising:
(a) a CDRL1 comprising RX¹SENIYX²X³LA (SEQ ID NO: 198), wherein
   X¹ is A or V;
   X² is S or N; and
   X³ is H, N, or Y;
(b) a CDRL2 comprising X¹AX²NLAX³ (SEQ ID NO: 199), wherein
   X¹ is A or N;
   X² is T or K; and
   X³ is N, L, Q, G, F, V, K, S, R, T, H, or P; and
(c) a CDRL3 comprising QX¹FX²GPPLT (SEQ ID NO: 200), wherein
   X¹ is L or H; and
   X² is W, F, Y, P, N, V, K, M, L, G, or S.

In some embodiments, the Ig light comprises a CDRL1 comprising SEQ ID NO: 204; a CDRL2 comprising SEQ ID NO: 205; and a CDRL3 comprising SEQ ID NO: 206.

In some embodiments, the immunoglobulin light chain polypeptide comprises the sequence:

(SEQ ID NO: 194)
X₁IQX₂TQSPSSLSASVGDRVTITCRX₃SENIYX₄X₅LAWYQQKX₆
GKAPKLLIYX₇AX₈NLAX₉GVPSRFSGSGSGTDX₁₀TLTISSLQPE
DFATYYCQX₁₁FX₁₂GPPLTFGGGTKVEIKR,

or at least the CDRs thereof, wherein
X¹ is A or D;
X² is L or M;
X³ is A or V;
X⁴ is S or N;
X⁵ is H, N, or Y;
X⁶ is P or Q;
X⁷ is A or N;
X⁸ is T or K;
X⁹ is N, L, Q, G, F, V, K, S, R, T, H, or P;
X¹⁰ is F or Y;
X¹¹ is L or H;
X¹² is W, F, Y, P, N, V, K, M, L, G, S.
   In some embodiments, the Ig light chain polypeptide comprises SEQ ID NO: 194, provided that it retains the same CDRs (CDR1, CDR2, and CDR3) of any one of SEQ ID NOs: 157-192.

According to one embodiment, the BTLA-binding agent comprises an immunoglobulin heavy chain variable region of SEQ ID NO: 144 or an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) thereto; or an immunoglobulin heavy chain variable region comprising at least the CDRs of SEQ ID NO: 144, wherein the CDR regions are as provided above (e.g., CDR1-SEQ ID NO: 201, CDR2-SEQ ID NO: 202, and CDR3-SEQ ID NO: 203) or as determined in accordance with any of the various known immunoglobulin numbering schemes (e.g., Kabat, Chothia, Martin (Enhanced Chothia), IGMT, or AHo); and an immunoglobulin light chain variable region of SEQ ID NO: 174 or an amino acid sequence with at least 80%, 85%, or 90% sequence identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) thereto, or an immunoglobulin light chain variable region comprising at least the CDRs of SEQ ID NO: 174; wherein the CDR regions are as provided above (e.g., CDR1-SEQ ID NO: 204, CDR2-SEQ ID NO: 205, and CDR3-SEQ ID NO: 206) or as determined in accordance with any of the various known immunoglobulin numbering schemes (e.g., Kabat, Chothia, Martin (Enhanced Chothia), IGMT, or AHo). In some embodiments, the antibody comprises a heavy chain variable region of SEQ ID NO: 144 and light chain variable region of SEQ ID NO: 174, or at least the CDRs thereof as determined by Kabat. In some embodiments, the antibody comprises a heavy chain variable region of SEQ ID NO: 144 and light chain variable region of SEQ ID NO: 174, or at least the CDRs thereof as determined by Chothia. In some embodiments, the antibody comprises a heavy chain variable region of SEQ ID NO: 144 and light chain variable region of SEQ ID NO: 174, or at least the CDRs thereof as determined by Martin. In some embodiments, the antibody comprises a heavy chain variable region of SEQ ID NO: 144 and light chain variable region of SEQ ID NO: 174, or at least the CDRs thereof as determined by IGMT. In some embodiments, the antibody comprises a heavy chain variable region of SEQ ID NO: 144 and light chain variable region of SEQ ID NO: 174, or at least the CDRs thereof as determined by AHo.

Also provided is a BTLA-binding agent that binds to the same epitope as a BTLA-binding agent comprising the immunoglobulin heavy and light chain polypeptides set forth herein. In some embodiments, the BTLA-binding agent binds to the same epitope as a BTLA binding agent comprising a heavy chain variable region of SEQ ID NO: 144 and a light chain variable region of SEQ ID NO: 174. In some embodiments, the BTLA binding agent binds to the same epitope as a BTLA binding agent comprising a heavy chain variable region of SEQ ID NO: 5 and a light chain variable region comprising SEQ ID NO: 17; a BTLA binding agent comprising a heavy chain variable region of SEQ ID NO: 207 and a light chain variable region comprising SEQ ID NO: 209; or a BTLA binding agent comprising a heavy chain variable region of SEQ ID NO: 217 and a light chain variable region comprising SEQ ID NO: 219. A BTLA-binding agent is considered to bind to the same epitope if it competes for binding to BTLA with a BTLA-binding agent comprising the immunoglobulin heavy and light chain polypeptides described herein. In some embodiments, provided herein is a BTLA binding agent that binds to amino acid residues 52-65 and/or 100-106 of human BTLA (e.g., SEQ ID NOs: 227 and/or 228) (reference sequence UniProt ID Q7Z6A9 or corresponding sequence positions of naturally occurring variant human BTLA). In some embodiments, provided herein is a BTLA binding agent that binds to amino acid residues 46-65, 82-91, or 100-106 of human BTLA (e.g., SEQ ID NOs: 229, 230, and/or 231) (reference sequence UniProt ID Q7Z6A9 or corresponding sequence positions of naturally occurring variant human BTLA).

Sequence “identity” as used in reference to nucleic acid or amino acid sequences can be determined by comparing a nucleic acid or amino acid sequence of interest to a reference nucleic acid or amino acid sequence. The percent identity is the percentage of nucleotides or amino acid residues that are the same (i.e., that are identical) as between the sequence of interest and the reference sequence when optimally aligned. A number of mathematical algorithms for obtaining the optimal alignment and calculating identity between two or more sequences are known and publically available. Examples of such programs include CLUSTAL-W, T-Coffee, and ALIGN (for alignment of nucleic acid and amino acid sequences), BLAST programs (e.g., BLAST 2.1, BL2SEQ, and later versions thereof operated by the National Center for Biotechnology Information, Bethesda, MD) and FASTA programs (e.g., FASTA3x, FASTM, and SSEARCH) (for sequence alignment and sequence similarity searches). Sequence alignment algorithms also are disclosed in, for example, Altschul et al., J. Molecular Biol., 215 (3): 403-410 (1990), Beigert et al., Proc. Natl. Acad. Sci. USA, 106 (10): 3770-3775 (2009), Durbin et al., eds., Biological Sequence Analysis: Probalistic Models of Proteins and Nucleic Acids, Cambridge University Press, Cambridge, UK (2009), Soding, Bioinformatics, 21 (7): 951-960 (2005), Altschul et al., Nucleic Acids Res., 25 (17): 3389-3402 (1997), and Gusfield, Algorithms on Strings, Trees and Sequences, Cambridge University Press, Cambridge UK (1997)).

With respect to sequences having less than 100% identity to the heavy and light chain sequences specifically set forth above, one or more amino acids of the aforementioned immunoglobulin heavy chain polypeptides and/or light chain polypeptides can be replaced or substituted with a different amino acid, and/or one of more amino acids can be deleted from or inserted into the disclosed amino acid sequences, provided the activity of the polypeptide (e.g., the ability to bind BTLA when present as part of an BTLA-binding agent) is substantially retained. The “biological activity” of a BTLA-binding agent refers to, for example, binding affinity for a particular BTLA epitope (without inhibiting BTLA-binding to its receptor and/or without inhibiting BTLA activity in vivo (e.g., IC₅₀)), pharmacokinetics, and cross-reactivity (e.g., with non-human homologs or orthologues of the BTLA protein, or with other proteins or tissues). In some embodiments, the biological activity of the BTLA-binding agent includes the ability of the agent to enhance BTLA-binding to its receptor(s) and/or otherwise increase BTLA activity in vivo. Other biological properties or characteristics of an antigen-binding agent recognized in the art include, for example, avidity, selectivity, solubility, folding, immunotoxicity, expression, and formulation. The aforementioned properties or characteristics can be observed, measured, and/or assessed using standard techniques including, but not limited to, ELISA, competitive ELISA, surface plasmon resonance analysis (BIACORE™), or solution phase competition (KINEXA™), in vitro or in vivo neutralization assays, receptor-ligand binding assays, cytokine or growth factor production and/or secretion assays, and signal transduction and immunohistochemistry assays.

An amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra).

Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gln), lysine (K or Lys), and arginine (R or Arg).

Aliphatic amino acids may be sub-divided into four sub-groups. The “large aliphatic non-polar sub-group” consists of valine, leucine, and isoleucine. The “aliphatic slightly-polar sub-group” consists of methionine, serine, threonine, and cysteine. The “aliphatic polar/charged sub-group” consists of glutamic acid, aspartic acid, asparagine, glutamine, lysine, and arginine. The “small-residue sub-group” consists of glycine and alanine. The group of charged/polar amino acids may be sub-divided into three sub-groups: the “positively-charged sub-group” consisting of lysine and arginine, the “negatively-charged sub-group” consisting of glutamic acid and aspartic acid, and the “polar sub-group” consisting of asparagine and glutamine.

Aromatic amino acids may be sub-divided into two sub-groups: the “nitrogen ring sub-group” consisting of histidine and tryptophan and the “phenyl sub-group” consisting of phenylalanine and tyrosine.

Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

The foregoing mutations (e.g., substitutions) may be made in any region of the Ig chain. In some embodiments, amino acid(s) are substituted in a CDR (e.g., CDR1, CDR2, or CDR3) of the immunoglobulin heavy chain polypeptide and/or light chain polypeptide; in other embodiments, the amino acid(s) are substituted in the framework regions and not the CDRs; in still other embodiments, the amino acids substituted in both the framework regions and CDRs. In some embodiments, the foregoing mutations are made in regions other than in the CDRs. In other words, the heavy chain variable region and light chain variable region can have the stated sequence identity to the sequences provided herein, but retain the CDRs of the specifically provided sequence.

In addition, one or more amino acids can be inserted into the aforementioned immunoglobulin heavy chain polypeptides and/or light chain polypeptides, provided it does not abrogate the function of the polypeptide in the context of the BTLA-binding agent (e.g., does not prevent a binding agent comprising the polypeptide from binding to BTLA without inhibiting BTLA from binding to its receptor). Any number of any suitable amino acids can be inserted into the amino acid sequence of the immunoglobulin heavy chain polypeptide and/or light chain polypeptide. In some embodiments, at least one amino acid (e.g., 2 or more, 5 or more, or 10 or more amino acids), but not more than 20 amino acids (e.g., 18 or less, 15 or less, or 12 or less amino acids), can be inserted into the amino acid sequence of the immunoglobulin heavy chain polypeptide and/or light chain polypeptide. In other embodiments, 1-10 amino acids (e.g., 1, 2, 3, 4, 5, 6, 7, 8, 9, or 10 amino acids) are inserted into the amino acid sequence of the immunoglobulin heavy chain polypeptide and/or light chain polypeptide. In this respect, the amino acid(s) can be inserted into any one of the aforementioned immunoglobulin heavy chain polypeptides and/or light chain polypeptides in any suitable location. In some embodiments, the amino acid(s) are inserted into a CDR (e.g., CDR1, CDR2, or CDR3) of the immunoglobulin heavy chain polypeptide and/or light chain polypeptide; in other embodiments, the amino acid(s) are inserted into the framework regions and not the CDRs; in still other embodiments, the amino acids are inserted in both the framework regions and CDRs.

The inventive isolated immunoglobulin heavy chain polypeptide and light chain polypeptides are not limited to polypeptides comprising the specific amino acid sequences described herein, and also includes any heavy chain polypeptide or light chain polypeptide that competes with the inventive immunoglobulin heavy chain polypeptide or light chain polypeptide for binding to BTLA when included in a BTLA-binding agent. In this respect, for example, the immunoglobulin heavy chain polypeptide or light chain polypeptide can be any heavy chain polypeptide or light chain polypeptide that binds to the same epitope of BTLA recognized by the heavy and light chain polypeptides described herein when included in a BTLA-binding agent. Antibody competition can be assayed using routine peptide competition assays, which utilize ELISA, Western blot, or immunohistochemistry methods (see, e.g., U.S. Pat. Nos. 4,828,981 and 8,568,992; and Braitbard et al., Proteome Sci., 4:12 (2006)).

The BTLA-binding agent is a proteinaceous molecule comprising the immunoglobulin heavy chain variable region and light chain variable region set forth herein that specifically binds to the BTLA protein (e.g., an antibody or an antigen-binding fragment thereof). In some embodiments, the BTLA-binding agent binds BTLA without abrogating or, in some embodiments, without inhibiting BTLA-binding to its receptor. In some embodiments, the BTLA-binding agent enhances binding of BTLA to HVEM so as to increase BTLA-mediated signaling. The term “inhibit” as used herein with respect to binding of BTLA to its receptor or BTLA-mediated signaling refers to the ability to substantially antagonize, prohibit, prevent, restrain, slow, disrupt, alter, eliminate, or stop (in whole or in part), the binding of BTLA to its receptor or BTLA-HVEM mediated signaling in the presence of the binding agent as compared to such binding or signaling in the absence of the binding agent. The term “increase” or “enhance” as used in reference to BTLA-binding to its receptor or BTLA-mediated signaling means to increase or enhance such binding or signaling in any way and to any degree in the presence of the binding agent as compared to such binding or signaling in the absence of the binding agent. In some embodiments, BTLA-binding to its receptor or BTLA-mediated signaling is increased sufficiently to reduce or alleviate any symptom of a disease or condition associated with deficient BTLA activity, or which benefits from enhanced BTLA activity, or to reverse the progression or severity of such a disease or condition. In some embodiments, the BTLA-binding agent does not inhibit BTLA-receptor binding by more than 25% (e.g., does not inhibit BTLA-receptor binding by more than 10% or more than 5%). In some embodiments, the BTLA-binding agent increases BTLA-receptor binding by at least about 20%, about 30%, about 40%, about 50%, about 60%, about 70%, about 80%, about 90%, about 95%, about 100%, or a range defined by any two of the foregoing values, as compared to the activity of BTLA in the absence of the BTLA-binding agent.

The BTLA-binding agent can be part of a multispecific (e.g., bispecific or “dual reactive”) construct (e.g., a multispecific antibody, such as a bispecific or dual reactive antibody) that binds BTLA and another antigen. Such a construct can comprise immunoglobulin heavy and light chain polypeptides that bind BTLA as described herein in combination with immunoglobulin heavy chains and light chains from an immunoglobulin that binds an antigen other than BTLA. Such a bispecific BTLA-binding agent can bind, for example, BTLA and another negative regulator of the immune system, such as cytotoxic T lymphocyte antigen-4 (CTLA-4), T Cell Immunoglobulin and Mucin Domain-3 (TIM-3), programmed death 1 (PD-1) and/or the Lymphocyte Activation Gene 3 protein (LAG-3). Immunoglobulins that bind such other target antigens are known in the art.

Antibody conjugates also are provided herein. For example, the BTLA-binding agent can be a conjugate of (1) an anti-BTLA antibody or fragment thereof, and (2) a secondary protein or non-protein moiety. By way of further illustration, the BTLA-binding agent can comprise an anti-BTLA antibody or fragment thereof conjugated to another peptide, a fluorescent molecule, or a chemotherapeutic agent.

In some embodiments, the BTLA-binding agent can be a “whole” immunoglobulin or an antigen-binding immunoglobulin “fragment.” A “whole” immunoglobulin typically consists of four polypeptides: two heavy (H) chain polypeptides and two light (L) chain polypeptides. Each of the heavy chains contains one N-terminal variable (V_{H}) region and three C-terminal constant (C_{H}1, C_{H}2, and C_{H}3) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. In a typical immunoglobulin, each light chain is linked to a heavy chain by disulfide bonds, and the two heavy chains are linked to each other by disulfide bonds. In this configuration, the light chain variable region is generally aligned with the variable region of the heavy chain, and the light chain constant region is generally aligned with the first constant region of the heavy chain. The remaining constant regions of the heavy chains are generally aligned with each other.

The variable regions or hypervariable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region, which are located between the hypervariable or complementary determining regions (CDRs). There are four framework regions in each variable domain, which are designated FR1, FR2, FR3, and FR4. The framework regions form the β sheets that provide the structural framework of the variable region (see, e.g., C. A. Janeway et al. (eds.), Immunobiology, 5th Ed., Garland Publishing, New York, NY (2001)). The framework regions are connected by three complementarity determining regions (CDRs). The three CDRs, known as CDR1, CDR2, and CDR3, form the “hypervariable region” of an antibody, which is generally considered to be responsible for antigen binding.

The term “antibody fragment” and like terms (e.g., “fragment of an antibody,” “antibody fragment,” “functional fragment of an antibody”) are used interchangeably herein to mean one or more fragments or portions of an antibody that retain the ability to specifically bind to an antigen (see, generally, Holliger et al., Nat. Biotech., 23 (9): 1126-1129 (2005)). Antibody “fragments,” as used herein and routinely in the art, include not only fragments or pieces of a whole antibody in the literal sense, but also other known engineered antibody-like constructs, which might include linkers or other elements that do not occur in a natural antibody in addition to a fragment of an antibody. The antibody fragment can comprise, for example, one or more (or all) of the CDRs, the variable regions (or portions thereof), the constant regions (or portions thereof), or some combination thereof. Examples of antibody fragments include, but are not limited to, (i) a Fab fragment, which is a monovalent fragment consisting of the V_{L}, V_{H}, C_{L}, and C_{H}1 domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a domain antibody (dAb), which is an antibody single variable region domain (V_{H} or V_{L}) polypeptide that specifically binds antigen. The BTLA-binding agent also can be a single chain antibody fragment. Examples of single chain antibody fragments include, but are not limited to, (i) a single chain Fv (scFv), which is a monovalent molecule consisting of the two domains of the Fv fragment (i.e., V_{L} and V_{H}) joined by a synthetic linker which enables the two domains to be synthesized as a single polypeptide chain (see, e.g., Bird et al., Science, 242:423-426 (1988); Huston et al., Proc. Natl. Acad. Sci. USA, 85:5879-5883 (1988); and Osbourn et al., Nat. Biotechnol., 16:778 (1998)) and (ii) a diabody, which is a dimer of polypeptide chains, wherein each polypeptide chain comprises a V_{H} connected to a V_{L} by a peptide linker that is too short to allow pairing between the V_{H} and V_{L} on the same polypeptide chain, thereby driving the pairing between the complementary domains on different V_{H}-V_{L} polypeptide chains to generate a dimeric molecule having two functional antigen binding sites. Antibody fragments are known in the art and are described in more detail in, e.g., U.S. Patent Application Publication 2009/0093024 A1.

In some embodiments, the BTLA-binding agent comprises a heavy chain constant region, such as a fragment crystallizable (Fc) region or portion thereof. The Fc region can be of any Ig class/subclass (IgA (IgA1, IgA2), IgD, IgE, IgG (IgG1, IgG2, IgG3 and IgG4), IgM, including variants thereof. In some embodiments, the BTLA-binding agent is a “whole” or “complete” Ig (i.e., an antibody). In some embodiments, the BTLA binding agent comprises an IgG Fc region, such as IgG1 or IgG4. For instance, the BLTA binding agent can be an IgG1 or IgG4 antibody. In some embodiments, the BTLA binding agent comprises a variable heavy chain region and variable light chain region comprising SEQ ID NOs: 144 and 174, respectively, or at least the CDRs thereof (as provided herein or as determined according to Kabat, Chothia, Martin (Enhanced Chothia), IGMT, or AHo numbering), or sequences comprising 90% identity thereto, wherein the BTLA binding agent is an IgG1 antibody. In some embodiments, the BTLA binding agent comprises a variable heavy chain region and variable light chain region comprising SEQ ID NOs: 144 and 174, respectively, or at least the CDRs thereof (as provided herein or as determined according to Kabat, Chothia, Martin (Enhanced Chothia), IGMT, or AHo numbering), or sequences comprising 90% identity thereto, wherein the BTLA binding agent is an IgG4 antibody.

The BTLA-binding agent can be, or can be obtained from, a human antibody, a non-human antibody, or a chimeric antibody. By “chimeric” is meant an antibody or fragment thereof comprising both human and non-human regions. Preferably, the BTLA-binding agent is a humanized antibody. A “humanized” antibody is a monoclonal antibody comprising a human antibody scaffold and at least one CDR obtained or derived from a non-human antibody. Non-human antibodies include antibodies isolated from any non-human animal, such as, for example, a rodent (e.g., a mouse or rat). A humanized antibody can comprise, one, two, or three CDRs obtained or derived from a non-human antibody. In one embodiment of the invention, CDRH3 of the inventive BTLA-binding agent is obtained or derived from a mouse monoclonal antibody, while the remaining variable regions and constant region of the inventive BTLA-binding agent are obtained or derived from a human monoclonal antibody.

A human antibody, a non-human antibody, a chimeric antibody, or a humanized antibody can be obtained by any means, including via in vitro sources (e.g., a hybridoma or a cell line producing an antibody recombinantly) and in vivo sources (e.g., rodents). Methods for generating antibodies are known in the art and are described in, for example, Köhler and Milstein, Eur. J. Immunol., 5:511-519 (1976); Harlow and Lane (eds.), Antibodies: A Laboratory Manual, CSH Press (1988); and Janeway et al. (eds.), Immunobiology, 5th Ed., Garland Publishing, New York, NY (2001)). In certain embodiments, a human antibody or a chimeric antibody can be generated using a transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the Medarex HUMAB-MOUSE™, the Kirin TC MOUSE™, and the Kyowa Kirin KM-MOUSE™ (see, e.g., Lonberg, Nat. Biotechnol., 23 (9): 1117-25 (2005), and Lonberg, Handb. Exp. Pharmacol., 181:69-97 (2008)). A humanized antibody can be generated using any suitable method known in the art (see, e.g., An, Z. (ed.), Therapeutic Monoclonal Antibodies: From Bench to Clinic, John Wiley & Sons, Inc., Hoboken, New Jersey (2009)), including, e.g., grafting of non-human CDRs onto a human antibody scaffold (see, e.g., Kashmiri et al., Methods, 36 (1): 25-34 (2005); and Hou et al., J. Biochem., 144 (1): 115-120 (2008)). In one embodiment, a humanized antibody can be produced using the methods described in, e.g., U.S. Patent Application Publication 2011/0287485 A1.

The BTLA-binding agent is not limited by any particular affinity to its epitope. The term “affinity” refers to the equilibrium constant for the reversible binding of two agents and is expressed as the dissociation constant (K_{D}). However, in some embodiments, the BTLA can have an affinity for BTLA of from about 1 picomolar (pM) to about 100 micromolar (μM) (e.g., from about 1 picomolar (pM) to about 1 nanomolar (nM), from about 1 nM to about 1 micromolar (μM), or from about 1 μM to about 100 μM). In one embodiment, the BTLA-binding agent can bind to an BTLA protein with a K_{D} less than or equal to 1 nanomolar (e.g., 0.9 nM, 0.8 nM, 0.7 nM, 0.6 nM, 0.5 nM, 0.4 nM, 0.3 nM, 0.2 nM, 0.1 nM, 0.05 nM, 0.025 nM, 0.01 nM, 0.001 nM, or a range defined by any two of the foregoing values). In another embodiment, the BTLA-binding agent can bind to BTLA with a K_{D} less than or equal to 200 pM (e.g., 190 pM, 175 pM, 150 pM, 125 pM, 110 pM, 100 pM, 90 pM, 80 pM, 75 pM, 60 pM, 50 pM, 40 pM, 30 pM, 25 pM, 20 pM, 15 pM, 10 pM, 5 pM, 1 pM, or a range defined by any two of the foregoing values). Immunoglobulin affinity for an antigen or epitope of interest can be measured using any art-recognized assay. Such methods include, for example, fluorescence activated cell sorting (FACS), separable beads (e.g., magnetic beads), surface plasmon resonance (SPR), solution phase competition (KINEXA™), antigen panning, competitive binding assays, and/or ELISA (see, e.g., Janeway et al. (eds.), Immunobiology, 5th ed., Garland Publishing, New York, NY, 2001). In some embodiments, the BTLA binding agent has an affinity to BTLA as described above (e.g., 1 nM or less or 200 pM or less) when measured using surface plasmon resonance (SPR). In some embodiments, the BTLA binding agent has an affinity to BTLA as described above (e.g., 1 nM or less or 200 pM or less) when measured using surface plasmon resonance (SPR).

The BTLA-binding agent provided herein can be used for any purpose, such as modulating (e.g., promoting or enhancing) BTLA signaling in a mammal, modulating (e.g., inhibiting) an immune response in a mammal, and/or treating or preventing a disease or condition associated with deficient BTLA signaling (e.g., associated with BTLA-mediated immunoresponse). Thus, in one aspect, the invention provides a method of promoting or enhancing BTLA signaling in a mammal comprising administering the BTLA-binding agent described herein to the mammal, whereby the BTLA-binding agent promotes or enhances binding of BTLA to HVEM or otherwise promotes or enhances BTLA signaling.

The mammal can be a mammal afflicted with a disease or condition associated with deficient BTLA signaling or associated with a BTLA-mediated immunoresponse. For instance, the mammal can be afflicted with a disease or condition that is improved by immunosuppression. Such a disease will be responsive to BTL agonism, such that administration of the BTLA-binding agent to the mammal will treat or prevent the disease or condition. A disease, condition, or disorder that is associated with BTLA signaling and responsive to BTLA agonism can be any disease or disorder in which an increase in BTLA activity has a therapeutic benefit in mammals, preferably humans, or the underexpression or decreased activity of BTLA causes or contributes to the pathological effects of the disease or disorder. Without wishing to be bound by any theory or mechanism of action, it is believed that the BTLA-binding agent facilitates immunosuppressive BTLA-HVEM signaling and, thus, suppresses immune response.

An “immune response” can entail, for example, antibody production and/or the activation of immune effector cells (e.g., T-cells), production of inflammatory cytokines, or any of the indications or disorders described herein or otherwise known in the art. As used herein, the terms “treatment,” “treating,” and the like refer to obtaining a desired pharmacologic and/or physiologic effect. Preferably, the effect is therapeutic, i.e., the effect partially or completely cures a disease and/or adverse symptom attributable to the disease. To this end, the inventive method comprises administering a “therapeutically effective amount” of the BTLA-binding agent. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the BTLA binding agent to elicit a desired response in the individual.

Alternatively, the pharmacologic and/or physiologic effect may be prophylactic, i.e., the effect completely or partially prevents a disease or symptom thereof. In this respect, the inventive method comprises administering a “prophylactically effective amount” of the BTLA binding agent. A “prophylactically effective amount” refers to an amount effective, at dosages and for periods of time necessary, to achieve a desired prophylactic result (e.g., prevention of disease onset).

The BTLA-binding agent is useful for suppressing an immune response and treating any disease or condition associated with an abnormal or excessive immune response. The disease or disorder can be an inflammatory or autoimmune disorder (e.g., a T-cell or B-cell driven inflammatory disease or condition). Examples of inflammatory or autoimmune disorders include, for example, infections (viral, bacterial, fungal and parasitic), endotoxic shock associated with infection, arthritis, rheumatoid arthritis, asthma, Chronic obstructive pulmonary disease (COPD), pelvic inflammatory disease, Behcet disease, Alzheimer's Disease, inflammatory bowel disease including Crohn's disease and ulcerative colitis, Peyronie's Disease, coeliac disease, gallbladder disease, Pilonidal disease, peritonitis, psoriasis, psoriatic arthritis, vasculitis, antineutrophil cytoplasmic antibody-associated (ANCA) vasculitis, surgical adhesions, stroke, Type I Diabetes, lyme disease, arthritis, meningoencephalitis, autoimmune uveitis, immune mediated inflammatory disorders of the central and peripheral nervous system such as multiple sclerosis, lupus (such as systemic lupus erythematosus and chronic discoid lupus erythematosus) and Guillain-Barr syndrome, Atopic dermatitis, polymyositis, dermatomyositis, autoimmune hepatitis, fibrosing alveolitis, Grave's disease, IgA nephropathy, idiopathic thrombocytopenia purpura, Meniere's disease, pemphigus, pemphigoid, primary biliary cholangitis, hepatitis, sarcoidosis, scleroderma (localized scleroderma, systemic scleroderma, and progressive systemic scleroderma), Ganulomatosis with polyangiitis, other autoimmune disorders, cholangitis, pancreatitis, trauma (surgery), graft-versus-host disease, transplant rejection, heart disease including ischaemic diseases such as myocardial infarction as well as atherosclerosis, periarteritis nodosa (polyarteritis nodosa and microscopic polyangiitis), allergic granulomatous angiitis, hypersensitivity angiitis, aortitis syndrome (Takayasu arteritis), temporal arteritis, intravascular coagulation, bone resorption, osteoporosis, osteoarthritis, periodontitis and hypochlorhydia, Still's disease, Cogan's syndrome, RS3PE, polymyalgia rheumatica, fibromyalgia syndrome, antiphospholipid antibody syndrome, eosinophilic fasciitis, Guillain-Barre syndrome, myasthenia gravis, chronic atrophic gastritis, Goodpasture's syndrome, rapidly progressive glomerulonephritis, megaloblastic anemia, hemolytic anemia, autoimmune neutropenia, Hashimoto's thyroiditis, autoimmune adrenal insufficiency, primary hypothyroidism, idiopathic Addison's disease (chronic adrenal insufficiency), herpes gestationis, linear IgA bullous skin disease, epidermolysis bullosa acquisita, alopecia areata, vitiligo, Harada disease, autoimmune optic neuropathy, idiopathic azoospermia, recurrent fetal loss, or infertility related to lack of fetal-maternal tolerance.

In some embodiments, the disease or disorder is arthritis (e.g., rheumatoid arthritis or TNF-refractory rheumatoid arthritis), giant cell arteritis, polymyalgia rheumatica, primary Sjögren's Syndrome, alopecia areata, primary biliary cholangitis (PBC), vitiligo, ANCA Vasculitis, Type 1 Diabetes, noninfectious uveitis psoriasis, graft vs. host disease (GvHD) or inflammatory bowel disease.

As used herein, the terms “treatment,” “treating,” and the like refer to obtaining a desired pharmacologic and/or physiologic effect. Preferably, the effect is therapeutic, i.e., the effect partially or completely cures a disease and/or alleviates to any degree an adverse symptom attributable to the disease. To this end, the inventive method comprises administering a “therapeutically effective amount” of the BTLA-binding agent. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the BTLA-binding agent to elicit a desired response in the individual. For example, a therapeutically effective amount of a BTLA-binding agent of the invention is an amount which increases BTLA bioactivity in a human.

Alternatively, the pharmacologic and/or physiologic effect may be prophylactic, i.e., the effect completely or partially prevents a disease or symptom thereof. In this respect, the inventive method comprises administering a “prophylactically effective amount” of the BTLA-binding agent. A “prophylactically effective amount” refers to an amount effective, at dosages and for periods of time necessary, to achieve a desired prophylactic result (e.g., prevention of disease onset).

In some embodiments, the method of treating an inflammatory disease or condition comprises administering the BTLA-binding agent in a dose of at least 7 mg weekly, such as at least 10 mg weekly (e.g., 20 mg-800 mg weekly, 70 mg-800 mg weekly, 70 mg to about 300 mg weekly, or 70 mg to about 200 mg weekly), wherein the dose is administered intravenously. In some embodiments, the method of treating an inflammatory disease or condition comprises administering the BTLA-binding agent in a dose of at least 20 mg weekly, such as 20 mg-800 mg weekly, or 70 mg-800 mg weekly (e.g., 70 mg to 300 mg weekly, or 70 mg to 200 mg weekly), wherein the dose is administered subcutaneously.

In some embodiments, the method comprises administering the BTLA-binding agent in a single intravenous dose of at least 150 mg, or at least 200 mg, wherein the dose is administered not more than once every 3 weeks or not more than once every 4 weeks (e.g., not more than once every 30 days not more than once every 60 days, not more than once every 90 days, not more than once every 120 days, not more than once every 150 days, or not more than once 180 days). In some embodiments, the method comprises administering the BTLA-binding agent in a single subcutaneous dose of at least 300 mg (e.g., 300 mg to 800 mg, or 300 mg to 500 mg), or at least 400 mg (e.g., 400 mg to 800 mg, or 400 mg to 500 mg), wherein the dose is administered not more than once every 3 weeks or not more than once every 4 weeks (e.g., not more than once every 30 days not more than once every 60 days, not more than once every 90 days, not more than once every 120 days, not more than once every 150 days, or not more than once 180 days). In some embodiments, the BTLA binding agent is administered in a dosage regimen (dosage amount and dosage interval, such as those disclosed above) sufficient for the BTLA binding agent to maintain BTLA receptor occupancy on CD19+ B-cells and/or CD3+ T-cells of greater than or equal to 90%. All dosing regimens can be continued as needed to achieve the desired clinical outcome.

The BTLA-binding agent can be part of a composition suitable for administration to a mammal. Preferably, the composition is a pharmaceutically acceptable (e.g., physiologically acceptable) composition, which comprises a carrier, preferably a pharmaceutically acceptable (e.g., physiologically acceptable) carrier, and the inventive amino acid sequences, antigen-binding agent, or vector. Any suitable carrier can be used within the context of the invention, and such carriers are well known in the art. The choice of carrier will be determined, in part, by the particular site to which the composition may be administered and the particular method used to administer the composition. The composition also can comprise any other excipient used in the formulation of therapeutic molecules (e.g., proteins or antibodies), particularly parenteral formulations, including, for instance, buffers, tonicity modifiers, stabilizers, surfactants and the like. The composition optionally can be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

Therapeutic or prophylactic efficacy can be monitored by periodic assessment of treated patients. For repeated administrations over several days or longer, depending on the condition, the treatment can be repeated until a desired suppression of disease symptoms occurs, or alternatively, the treatment can be continued for the lifetime of the patient. However, other dosage regimens may be useful and are within the scope of the invention. The desired dosage can be delivered by a single bolus administration of the composition, by multiple bolus administrations of the composition, or by continuous infusion administration of the composition.

Administration may be effected using any standard administration techniques, including oral, intravenous, intraperitoneal, subcutaneous, pulmonary, transdermal, intramuscular, intranasal, buccal, sublingual, or suppository administration. The composition preferably is suitable for parenteral administration. The term “parenteral,” as used herein, includes intravenous, intramuscular, subcutaneous, rectal, vaginal, and intraperitoneal administration. More preferably, the composition is administered to a mammal using peripheral systemic delivery by intravenous, intraperitoneal, or subcutaneous injection.

Once administered to a mammal (e.g., a human), the biological activity of the BTLA-binding agent can be measured by any suitable method known in the art. The biological activity may be correlated to the stability of the BTLA-binding agent in the body. In one embodiment of the invention, the BTLA-binding agent (e.g., an antibody) has an in vivo half-life between about 30 minutes and 45 days (e.g., about 30 minutes, about 45 minutes, about 1 hour, about 2 hours, about 4 hours, about 6 hours, about 10 hours, about 12 hours, about 1 day, about 5 days, about 10 days, about 15 days, about 25 days, about 35 days, about 40 days, about 45 days, or a range defined by any two of the foregoing values). In another embodiment, the BTLA-binding agent has an in vivo half-life between about 2 hours and 20 days (e.g., about 5 hours, about 10 hours, about 15 hours, about 20 hours, about 2 days, about 3 days, about 7 days, about 12 days, about 14 days, about 17 days, about 19 days, or a range defined by any two of the foregoing values). In another embodiment, the BTLA-binding agent has an in vivo half-life between about 10 days and about 40 days (e.g., about 10 days, about 13 days, about 16 days, about 18 days, about 20 days, about 23 days, about 26 days, about 29 days, about 30 days, about 33 days, about 37 days, about 38 days, about 39 days, about 40 days, or a range defined by any two of the foregoing values).

The BTLA-binding agent of the invention may be administered alone or in combination with other drugs. For example, the BTLA-binding agent can be administered in combination with other agents for the treatment or prevention of the diseases disclosed herein, such as other agents anti-inflammatory or immunosuppressive agents. In this respect, for example, the BTLA-binding agent can be used in combination with at least one other agent including, for example, any anti-inflammatory agent known in the art, glucocorticoids, small molecule immunosuppressive agents, vaccines, biological therapies (e.g., other monoclonal antibodies, viruses, gene therapy, and adoptive T-cell transfer), and/or surgery. When the inventive method treats an infectious disease, the BTLA-binding agent can be administered in combination with at least one anti-bacterial agent or at least one anti-viral agent. In this respect, the anti-bacterial agent can be any suitable antibiotic known in the art. The anti-viral agent can be any vaccine of any suitable type that specifically targets a particular virus (e.g., live-attenuated vaccines, subunit vaccines, recombinant vector vaccines) and any small molecule anti-viral therapies (e.g., viral replication inhibitors and nucleoside analogs).

In addition to therapeutic uses, the BTLA-binding agent described herein can be used in diagnostic or research applications. In this respect, the BTLA-binding agent can be used in a method to diagnose a disorder or disease in which the improper expression (e.g., underexpression) or decreased activity of BTLA causes or contributes to the pathological effects of the disease or disorder. In a similar manner, the BTLA-binding agent can be used in an assay to monitor BTLA protein levels in a subject being tested for a disease or disorder that is responsive to BTLA promotion. Research applications include, for example, methods that utilize the BTLA-binding agent and a label to detect a BTLA protein in a sample, e.g., in a human body fluid or in a cell or tissue extract. The BTLA-binding agent can be used with or without modification, such as covalent or non-covalent labeling with a detectable moiety. For example, the detectable moiety can be a radioisotope (e.g., ³H, ¹⁴C, ³²P, ³⁵S, or ¹²⁵I), a fluorescent or chemiluminescent compound (e.g., fluorescein isothiocyanate, rhodamine, or luciferin), an enzyme (e.g., alkaline phosphatase, beta-galactosidase, or horseradish peroxidase), or prosthetic groups. Any method known in the art for separately conjugating an antigen-binding agent (e.g., an antibody) to a detectable moiety may be employed in the context of the invention (see, e.g., Hunter et al., Nature, 194:495-496 (1962); David et al., Biochemistry, 13:1014-1021 (1974); Pain et al., J. Immunol. Meth., 40:219-230 (1981); and Nygren, J. Histochem. and Cytochem., 30:407-412 (1982)).

BTLA protein levels can be measured using the BTLA-binding agent provided herein by any suitable method known in the art. Such methods include, for example, radioimmunoassay (RIA), and FACS. Normal or standard expression values of BTLA can be established using any suitable technique, e.g., by combining a sample comprising, or suspected of comprising, BTLA with a BTLA-specific antibody under conditions suitable to form an antigen-antibody complex. The antibody is directly or indirectly labeled with a detectable substance to facilitate detection of the bound or unbound antibody. Suitable detectable substances include various enzymes, prosthetic groups, fluorescent materials, luminescent materials, and radioactive materials (see, e.g., Zola, Monoclonal Antibodies: A Manual of Techniques, CRC Press, Inc. (1987)). The amount of BTLA polypeptide expressed in a sample is then compared with a standard value.

Without wishing to be bound by any particular theory or mechanism of action, it is believed that administration of a BTLA binding agent as described herein causes a portion of BTLA to be shed from at least some of the cells on which it is expressed, resulting in an increase in soluble BTLA (sBTLA) present in the blood, plasma, serum, or tissue (e.g., skin tissue) of the subject to which the BTLA binding agent is administered. Thus, provided herein is a method (e.g., an in vitro method) of detecting, measuring, or monitoring the pharmacological activity of a BTLA binding agent in a subject, the method comprising detecting soluble BTLA (sBTLA) in a sample of blood, plasma, serum, or tissue (e.g., skin tissue) from a subject to whom a BTLA binding agent has been administered. Also provided herein is a method of selecting a patient for treatment with a BTLA binding agent by detecting or measuring (e.g., in vitro) sBTLA in the blood, plasma, serum, or tissue (e.g., skin tissue) sample from a patient, wherein the patient is selected for treatment when the sBTLA levels in the patient (sample from the patient) are decreased as compared to the sBTLA levels of a normal, non-diseased subject of the same type (e.g., human subject). The sBTLA can be detected and, optionally, quantified using any of several techniques known in the art. In some embodiments, the sBTLA is detected by contacting a blood, plasma, serum, or tissue sample from the subject (e.g., in vitro) with a BTLA binding agent provided herein. In some embodiments, the sBTLA detected in the blood, plasma, serum, or tissue sample of the subject is bound to the BTLA binding agent (e.g., a previously administered BTLA binding agent).

In some embodiments, the BTLA binding agent administered to the subject, which induces shedding of BTLA in the subject, is a BTLA binding agent that does not inhibit binding of BTLA to HVEM. The BTLA binding agent can be any BTLA binding agent, such as any of the BTLA binding agents described herein. In some embodiments, the BTLA binding agent comprises the immunoglobulin heavy chain variable region of any one of SEQ ID NOs: 1-15, 207, 208, 217, or 218, or at least the CDRs thereof, or an amino acid sequence with at least 90% sequence identity thereto; and an immunoglobulin light chain variable region of any of SEQ ID NOs: 16-25, 209, 210, 219, or 220, or at least the CDRs thereof; or an amino acid sequence with at least 90% sequence identity thereto. For instance, the BTLA binding agent can comprise CDRs represented by SEQ ID NOs: 27, 30, 32, 36, 39, and 41. In other embodiments, the BTLA binding agent comprises the immunoglobulin heavy chain variable region of any one of SEQ ID NOs: 43-156, or at least the CDRs thereof, or an amino acid sequence with at least 90% sequence identity thereto; and an immunoglobulin light chain variable region of any of SEQ ID NOs: 157-192, or at least the CDRs thereof; or an amino acid sequence with at least 90% sequence identity thereto; having any or all of the other features described herein. For instance, the BTLA binding agent can comprise CDRs represented by SEQ ID NOs: 195-200; for example, a BTLA binding agent comprising CDRs comprising SEQ ID NOs: 201-206, or comprising Ig heavy and light chains of SEQ ID NOs: 193 and 194 or SEQ ID NOs: 144 and 174, or at least the CDRs thereof, or Ig heavy and light chains with 90% or more sequence identity to SEQ ID NOs: 193 and 194 or SEQ ID NOs: 144 and 174, respectively.

In some embodiments, sBTLA in the blood, plasma, serum, or tissue (e.g., skin tissue) of the subject to whom BTLA has been administered is detected using a capture antibody that binds to sBTLA. The capture antibody can be any antibody that binds to BTLA. In some embodiments, the capture antibody is a BTLA antibody provided herein. The capture antibody can be the same or different from the BTLA binding agent administered to the subject. In some embodiments, the sBTLA capture antibody is different from the BTLA binding agent administered to the subject. In some embodiments, the capture antibody comprises a heavy chain variable region comprising any one of SEQ ID NOs: 1-15, 207, 208, 217, or 218, or at least the CDRs thereof, or an amino acid sequence with at least 90% sequence identity thereto; and a light chain variable region comprising any one of SEQ ID NOs: 16-25, 209, 210, 219, or 220, or at least the CDRs thereof, or an amino acid sequence with at least 90% sequence identity thereto; having any or all of the other features described herein. In further embodiments, the capture antibody can comprise heavy chain CDRs 1-3 represented by SEQ ID NOs: 27, 30, and 32; and light chain CDRs 1-3 represented by SEQ ID NOs: 36, 39, and 41; or any of the more specifically recited CDRs provided herein. For example, the capture antibody can comprise an Ig heavy chain of SEQ ID NO: 26 (e.g., any of SEQ ID NOs: 1-15, 207, 208, 217, or 218), and an Ig light chain of SEQ ID NO: 35 (e.g., any of SEQ ID NOs: 16-25, 209, 210, 219, or 220). Optionally, in combination with such embodiments, the BTLA binding agent administered to the subject can comprise the immunoglobulin heavy chain variable region of any one of SEQ ID NOs: 43-156, or at least the CDRs thereof, or an amino acid sequence with at least 90% sequence identity thereto; and an immunoglobulin light chain variable region of any of SEQ ID NOs: 157-192, or at least the CDRs thereof; or an amino acid sequence with at least 90% sequence identity thereto; having any or all of the other features described herein. For instance, the BTLA binding agent can comprise CDRs represented by SEQ ID NOs: 195-200; for example, a BTLA binding agent comprising CDRs comprising SEQ ID NOs: 201-206, or comprising Ig heavy and light chains of SEQ ID NOs: 193 and 194 or SEQ ID NOs: 144 and 174.

Without wishing to be bound by any particular theory or mechanism of action, it is believed that the binding of BTLA (or sBTLA) to an antibody that does not inhibit binding of BTLA to HVEM, as described herein, enhances the binding of the capture antibody to sBTLA. Thus, also provided herein is an assay for detecting and/or quantifying sBTLA in blood, plasma, serum, or tissue (e.g., skin tissue), the method comprising contacting a blood, plasma, serum, or tissue (e.g., skin tissue) sample with a capture antibody and a BTLA binding agent that does not inhibit binding of BTLA to HVEM. In some embodiments, the BTLA binding agent that does not inhibit binding of BTLA to HVEM is a BTLA binding agent that comprises the immunoglobulin heavy chain variable region of any one of SEQ ID NOs: 43-156, or at least the CDRs thereof; or an amino acid sequence with at least 90% sequence identity thereto; and an immunoglobulin light chain variable region of any of SEQ ID NOs: 157-192, or at least the CDRs thereof; or an amino acid sequence with at least 90% sequence identity thereto; having any or all of the other features described herein. For instance, the BTLA binding agent can comprise CDRs represented by SEQ ID NOs: 195-200; for example, a BTLA binding agent comprising CDRs comprising SEQ ID NOs: 201-206, or comprising Ig heavy and light chains of SEQ ID NOs: 193 and 194 or SEQ ID NOs: 144 and 174. In some embodiments, the capture antibody comprises a heavy chain variable region comprising any one of SEQ ID NOs: 1-15, 207, 208, 217, or 218, or at least the CDRs thereof, or an amino acid sequence with at least 90% sequence identity thereto; and a light chain variable region comprising any one of SEQ ID NOs: 16-25, 209, 210, 219, or 220, or at least the CDRs thereof, or an amino acid sequence with at least 90% sequence identity thereto; having any or all of the other features described herein. For instance, the capture antibody can comprise heavy chain CDRs 1-3 represented by SEQ ID NOs: 27, 30, and 32; and light chain CDRs 1-3 represented by SEQ ID NOs: 36, 39, and 41; or any of the more specifically recited CDRs provided herein. For example, the capture antibody can comprise an Ig heavy chain of SEQ ID NO: 26 (e.g., any of SEQ ID NOs: 1-15, 207, 208, 217, or 218), and an Ig light chain of SEQ ID NO: 35 (e.g., any of SEQ ID NOs: 16-25, 209, 210, 219, or 220).

In some embodiments of the foregoing methods of detecting, measuring, or qualifying sBTLA in blood, plasma, serum, or tissue, the method can further comprise comparing the concentration of sBTLA in the blood, plasma, serum, or tissue sample to a reference sBTLA concentration. Any suitable reference concentration can be used. In some embodiments, the reference sBTLA concentration is the concentration of sBTLA in a blood, plasma, serum, or tissue sample from the same patient or subject prior to administration of the BTLA binding agent. Alternatively, or in addition, the reference sBTLA concentration can be provided by the concentration of sBTLA in the blood, plasma, serum, or tissue of another subject, for instance, a normal, non-diseased subject of the same type that has not received administration of a BTLA binding agent, or a reference sBTLA concentration established by the statistical analysis of the sBTLA concentration in the blood, plasma, serum, or tissue of a population of such subjects (e.g., the mean concentration of sBTLA in blood, plasma, serum, or tissue samples from such a population of normal, non-diseased subjects not undergoing treatment with a BTLA binding agent). In some embodiments, the reference sBTLA concentration is established by a method comprising contacting a blood, plasma, serum, or tissue sample of the subject or blood, plasma, serum, or tissue samples from a population of subjects with a capture antibody as described above; optionally also contacting the blood, plasma, serum, or tissue sample with a BTLA binding agent that does not inhibit BTLA binding to HVEM as described herein, simultaneously or sequentially in any order.

In other embodiments, the method comprises comparing the concentration of sBTLA in sample of blood, plasma, serum, or tissue sample from a subject to whom a BTLA binding agent has been administered with the concentration of sBTLA in a sample of blood, plasma, serum, or tissue from the same subject at a different point in time, either before or after the BTLA binding agent had been administered to the subject. For instance, sBTLA concentration can be measured at two or more time points after administration of the BTLA binding agent and compared to evaluate the effect of the BTLA binding agent over time, optionally with one or more additional intervening administrations of the BTLA binding agent. In this manner, treatment with a BTLA binding agent can be monitored.

Further provided herein is a composition comprising a BTLA binding agent that does not inhibit binding of BTLA to HVEM, and a second capture antibody that binds to sBTLA, which is useful in the foregoing methods of detecting, measuring, or monitoring sBTLA in blood, plasma, serum, or tissue. In some embodiments, the BTLA binding agent that does not inhibit binding of BTLA to HVEM comprises the immunoglobulin heavy chain variable region of any one of SEQ ID NOs: 43-156, or at least the CDRs thereof; or an amino acid sequence with at least 90% sequence identity thereto; and an immunoglobulin light chain variable region of any of SEQ ID NOs: 157-192, or at least the CDRs thereof; or an amino acid sequence with at least 90% sequence identity thereto; having any or all of the other features described herein. For instance, the BTLA binding agent can comprise CDRs represented by SEQ ID NOs: 195-200; for example, a BTLA binding agent comprising CDRs comprising SEQ ID NOs: 201-206, or comprising Ig heavy and light chains of SEQ ID NOs: 193 and 194 or SEQ ID NOs: 144 and 174. In some embodiments, the capture antibody comprises a heavy chain variable region comprising any one of SEQ ID NOs: 1-15, 207, 208, 217, or 218, or at least the CDRs thereof, or an amino acid sequence with at least 90% sequence identity thereto; and a light chain variable region comprising any one of SEQ ID NOs: 16-25, 209, 210, 219, or 220, or at least the CDRs thereof, or an amino acid sequence with at least 90% sequence identity thereto; having any or all of the other features described herein. For instance, the capture antibody can comprise heavy chain CDRs 1-3 represented by SEQ ID NOs: 27, 30, and 32; and light chain CDRs 1-3 represented by SEQ ID NOs: 36, 39, and 41; or any of the more specifically recited CDRs provided herein. For example, the capture antibody can comprise an Ig heavy chain of SEQ ID NO: 26 (e.g., any of SEQ ID NOs: 1-15, 207, 208, 217, or 218), and an Ig light chain of SEQ ID NO: 35 (e.g., any of SEQ ID NOs: 16-25, 209, 210, 219, or 220).

The BTLA-binding agent, capture antibody, or composition can be provided in a kit, i.e., a packaged combination of reagents in predetermined amounts with instructions for performing a diagnostic assay. If the BTLA-binding agent is labeled with an enzyme, the kit desirably includes substrates and cofactors required by the enzyme (e.g., a substrate precursor which provides a detectable chromophore or fluorophore). In addition, other additives may be included in the kit, such as stabilizers, buffers (e.g., a blocking buffer or lysis buffer), and the like. The relative amounts of the various reagents can be varied to provide for concentrations in solution of the reagents which substantially optimize the sensitivity of the assay. The reagents may be provided as dry powders (typically lyophilized), including excipients which on dissolution will provide a reagent solution having the appropriate concentration.

### Nucleic Acids

The invention also provides one or more nucleic acids that encode the immunoglobulin heavy chain polypeptide, the immunoglobulin light chain polypeptide, and the BTLA-binding agent provided herein.

The term “nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to, methylated and/or capped polynucleotides. Nucleic acids are typically linked via phosphate bonds to form nucleic acid sequences or polynucleotides, though many other linkages are known in the art (e.g., phosphorothioates, boranophosphates, and the like).

The nucleic acid encoding the immunoglobulin heavy chain polypeptide, the immunoglobulin light chain polypeptide, or the BTLA-binding agent can be part of a vector. The vector can be, for example, a plasmid, episome, cosmid, viral vector (e.g., retroviral or adenoviral), or phage. Suitable vectors and methods of vector preparation are well known in the art (see, e.g., Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994)).

The vector typically comprises expression control sequences, such as a promoter, enhancer, polyadenylation signal, transcription terminator, signal peptide (e.g., the osteonectin signal peptide), internal ribosome entry site (IRES), and the like, that provide for the expression of the coding sequence in a host cell. Exemplary expression control sequences are known in the art and described in, for example, Goeddel, Gene Expression Technology: Methods in Enzymology, Vol. 185, Academic Press, San Diego, Calif. (1990).

A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, the RSV promoter. Inducible promoters include, for example, the Tet system (U.S. Pat. Nos. 5,464,758 and 5,814,618), the Ecdysone inducible system (No et al., Proc. Natl. Acad. Sci., 93:3346-3351 (1996)), the T-REX™ system (Invitrogen, Carlsbad, CA), LACSWITCH™ system (Stratagene, San Diego, CA), and the Cre-ERT tamoxifen inducible recombinase system (Indra et al., Nuc. Acid. Res., 27:4324-4327 (1999); Nuc. Acid. Res., 28: e99 (2000); U.S. Pat. No. 7,112,715; and Kramer & Fussenegger, Methods Mol. Biol., 308:123-144 (2005)).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences.

The vector also can comprise a “selectable marker gene.” The term “selectable marker gene,” as used herein, refers to a nucleic acid sequence that allow cells expressing the nucleic acid sequence to be specifically selected for or against, in the presence of a corresponding selective agent. Suitable selectable marker genes are known in the art and described in, e.g., International Patent Application Publications WO 1992/008796 and WO 1994/028143; Wigler et al., Proc. Natl. Acad. Sci. USA, 77:3567-3570 (1980); O'Hare et al., Proc. Natl. Acad. Sci. USA, 78:1527-1531 (1981); Mulligan & Berg, Proc. Natl. Acad. Sci. USA, 78:2072-2076 (1981); Colberre-Garapin et al., J. Mol. Biol., 150:1-14 (1981); Santerre et al., Gene, 30:147-156 (1984); Kent et al., Science, 237:901-903 (1987); Wigler et al., Cell, 11:223-232 (1977); Szybalska & Szybalski, Proc. Natl. Acad. Sci. USA, 48:2026-2034 (1962); Lowy et al., Cell, 22:817-823 (1980); and U.S. Pat. Nos. 5,122,464 and 5,770,359.

In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11:1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, CA) and pBK-CMV from Stratagene (La Jolla, CA) represent non-limiting examples of an episomal vector that uses T-antigen and the SV40 origin of replication in lieu of EBNA1 and oriP.

Other suitable vectors include integrating expression vectors, which may randomly integrate into the host cell's DNA, or may include a recombination site to enable the specific recombination between the expression vector and the host cell's chromosome. Such integrating expression vectors may utilize the endogenous expression control sequences of the host cell's chromosomes to effect expression of the desired protein. Examples of vectors that integrate in a site specific manner include, for example, components of the flp-in system from Invitrogen (Carlsbad, CA) (e.g., pcDNA™5/FRT), or the cre-lox system, such as can be found in the pExchange-6 Core Vectors from Stratagene (La Jolla, CA). Examples of vectors that randomly integrate into host cell chromosomes include, for example, pcDNA3.1 (when introduced in the absence of T-antigen) from Life Technologies (Carlsbad, CA), UCOE from Millipore (Billerica, MA), and pCI or pFN10A (ACT) FLEXI™ from Promega (Madison, WI).

Viral vectors also can be used. Representative commercially available viral expression vectors include, but are not limited to, the adenovirus-based Per.C6 system available from Crucell, Inc. (Leiden, The Netherlands), the lentiviral-based pLP1 from Invitrogen (Carlsbad, CA), and the retroviral vectors pFB-ERV plus pCFB-EGSH from Stratagene (La Jolla, CA).

Nucleic acid sequences encoding the inventive amino acid sequences can be provided to a cell on the same vector (i.e., in cis). A unidirectional promoter can be used to control expression of each nucleic acid sequence. In another embodiment, a combination of bidirectional and unidirectional promoters can be used to control expression of multiple nucleic acid sequences. Nucleic acid sequences encoding the inventive amino acid sequences alternatively can be provided to the population of cells on separate vectors (i.e., in trans). Each of the nucleic acid sequences in each of the separate vectors can comprise the same or different expression control sequences. The separate vectors can be provided to cells simultaneously or sequentially

The vector(s) comprising the nucleic acid(s) encoding the inventive amino acid sequences can be introduced into a host cell that is capable of expressing the polypeptides encoded thereby, including any suitable prokaryotic or eukaryotic cell. As such, the invention provides an isolated cell comprising the inventive vector. Preferred host cells are those that can be easily and reliably grown, have reasonably fast growth rates, have well characterized expression systems, and can be transformed or transfected easily and efficiently.

Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Erwinia. Particularly useful prokaryotic cells include the various strains of Escherichia coli (e.g., K12, HB101 (ATCC No. 33694), DH5α, DH10, MC1061 (ATCC No. 53338), and CC102).

In some embodiments, the vector is introduced into a eukaryotic cell. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Preferred yeast cells include, for example, Saccharomyces cerevisae and Pichia pastoris.

Suitable insect cells are described in, for example, Kitts et al., Biotechniques, 14:810-817 (1993); Lucklow, Curr. Opin. Biotechnol., 4:564-572 (1993); and Lucklow et al., J. Virol., 67:4566-4579 (1993). Preferred insect cells include Sf-9 and HI5 (Invitrogen, Carlsbad, CA).

In some embodiments, mammalian cells are utilized in the invention. A number of suitable mammalian host cells are known in the art, and many are available from the American Type Culture Collection (ATCC, Manassas, VA). Examples of suitable mammalian cells include, but are not limited to, Chinese hamster ovary cells (CHO) (e.g., CHO-K1 cells, ATCC No. CCL61), CHO DHFR-cells (Urlaub et al., Proc. Natl. Acad. Sci. USA, 97:4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, mouse L-929 cells, and BHK or Hak hamster cell lines, all of which are available from the ATCC. Methods for selecting suitable mammalian host cells and methods for transformation, culture, amplification, screening, and purification of cells are known in the art.

In one embodiment, the mammalian cell is a human cell. For example, the mammalian cell can be a human lymphoid or lymphoid derived cell line, such as a cell line of pre-B lymphocyte origin. Examples of human lymphoid cells lines include, without limitation, RAMOS (CRL-1596), Daudi (CCL-213), EB-3 (CCL-85), DT40 (CRL-2111), 18-81 (Jack et al., Proc. Natl. Acad. Sci. USA, 85:1581-1585 (1988)), Raji cells (CCL-86), PER. C6 cells (Crucell Holland B. V., Leiden, The Netherlands), and derivatives thereof.

A nucleic acid sequence encoding the inventive amino acid sequence may be introduced into a cell by any suitable method, such as by “transfection,” “transformation,” or “transduction.” “Transfection,” “transformation,” or “transduction,” as used herein, refer to the introduction of one or more exogenous polynucleotides into a host cell by using physical or chemical methods. Many suitable techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346:776-777 (1990)); and strontium phosphate DNA co-precipitation (Brash et al., Mol. Cell Biol., 7:2031-2034 (1987)). Phage or viral vectors can be introduced into host cells, after growth of infectious particles in suitable packaging cells, many of which are commercially available.

The nucleic acids and cells can be used for any purpose, such as for the manufacture of the BTLA-binding agent described herein. In this respect, the invention provides a method of preparing the BTLA-binding agent comprising culturing a cell comprising a nucleic acid encoding the heavy and/or light immunoglobulin polypeptides of the BTLA-binding agent. Phrased differently, the method comprises expressing a nucleic acid encoding the immunoglobulin heavy and/or light chains of the BTLA-binding agent in a cell (e.g., an in vitro cell, such as any of the cell lines discussed herein including CHO and CHO-K1 cells). It will be appreciated that the immunoglobulin heavy and light chains can be expressed from a single nucleic acid in a given cell, or the immunoglobulin heavy and light chains can be expressed from separate nucleic acids in the same cell. The method can further comprise harvesting and/or purifying the BTLA-binding agent from the cell or cell culture media using known techniques.

The following examples further illustrate the invention but, of course, should not be construed as in any way limiting its scope.

### EXAMPLES

Table 2 provides Examples of BTLA binding agents (e.g., antibodies or antibody fragments) comprising immunoglobulin heavy chain variable regions of SEQ ID NOs: 1-15 and light chain variable regions of SEQ ID NOs: 16-25.

TABLE 2

| | Heavy Chain | Light Chain |
|---|---|---|
| Ab Identification | Variable Region | Variable Region |
| APE08678 | SEQ ID NO: 1 | SEQ ID NO: 16 |
| APE08729 | SEQ ID NO: 2 | SEQ ID NO: 16 |
| APE08730 | SEQ ID NO: 1 | SEQ ID NO: 18 |
| APE08731 | SEQ ID NO: 1 | SEQ ID NO: 17 |
| APE08756 | SEQ ID NO: 4 | SEQ ID NO: 18 |
| APE08757 | SEQ ID NO: 4 | SEQ ID NO: 16 |
| APE08758 | SEQ ID NO: 3 | SEQ ID NO: 18 |
| APE08759 | SEQ ID NO: 3 | SEQ ID NO: 16 |
| APE08793 | SEQ ID NO: 4 | SEQ ID NO: 17 |
| APE08794 | SEQ ID NO: 3 | SEQ ID NO: 17 |
| APE08795 | SEQ ID NO: 2 | SEQ ID NO: 18 |
| APE08796 | SEQ ID NO: 2 | SEQ ID NO: 17 |
| APE09032 | SEQ ID NO: 2 | SEQ ID NO: 19 |
| APE09033 | SEQ ID NO: 1 | SEQ ID NO: 19 |
| APE09065 | SEQ ID NO: 1 | SEQ ID NO: 21 |
| APE09066 | SEQ ID NO: 1 | SEQ ID NO: 20 |
| APE09067 | SEQ ID NO: 7 | SEQ ID NO: 17 |
| APE09068 | SEQ ID NO: 6 | SEQ ID NO: 17 |
| APE09069 | SEQ ID NO: 5 | SEQ ID NO: 17 |
| APE09356 | SEQ ID NO: 10 | SEQ ID NO: 21 |
| APE09357 | SEQ ID NO: 10 | SEQ ID NO: 17 |
| APE09358 | SEQ ID NO: 9 | SEQ ID NO: 17 |
| APE09359 | SEQ ID NO: 8 | SEQ ID NO: 21 |
| APE09360 | SEQ ID NO: 8 | SEQ ID NO: 17 |
| APE09361 | SEQ ID NO: 7 | SEQ ID NO: 21 |
| APE09362 | SEQ ID NO: 5 | SEQ ID NO: 21 |
| APE09491 | SEQ ID NO: 5 | SEQ ID NO: 19 |
| APE09495 | SEQ ID NO: 11 | SEQ ID NO: 19 |
| APE09496 | SEQ ID NO: 11 | SEQ ID NO: 17 |
| APE09785 | SEQ ID NO: 13 | SEQ ID NO: 19 |
| APE09786 | SEQ ID NO: 13 | SEQ ID NO: 17 |
| APE09787 | SEQ ID NO: 12 | SEQ ID NO: 19 |
| APE09788 | SEQ ID NO: 12 | SEQ ID NO: 17 |
| APE09833 | SEQ ID NO: 8 | SEQ ID NO: 19 |
| APE09834 | SEQ ID NO: 8 | SEQ ID NO: 23 |
| APE09835 | SEQ ID NO: 8 | SEQ ID NO: 22 |
| APE09836 | SEQ ID NO: 5 | SEQ ID NO: 23 |
| APE09837 | SEQ ID NO: 5 | SEQ ID NO: 22 |
| APE09838 | SEQ ID NO: 2 | SEQ ID NO: 23 |
| APE09839 | SEQ ID NO: 2 | SEQ ID NO: 22 |
| APE09840 | SEQ ID NO: 15 | SEQ ID NO: 17 |
| APE09841 | SEQ ID NO: 15 | SEQ ID NO: 19 |
| APE09842 | SEQ ID NO: 14 | SEQ ID NO: 17 |
| APE09843 | SEQ ID NO: 14 | SEQ ID NO: 19 |
| APE09897 | SEQ ID NO: 8 | SEQ ID NO: 25 |
| APE09898 | SEQ ID NO: 8 | SEQ ID NO: 24 |
| APE09899 | SEQ ID NO: 5 | SEQ ID NO: 25 |
| APE09900 | SEQ ID NO: 5 | SEQ ID NO: 24 |
| APE09901 | SEQ ID NO: 2 | SEQ ID NO: 25 |
| APE09902 | SEQ ID NO: 2 | SEQ ID NO: 24 |

For antibodies or antibody fragments in Table 2, Kabat numbered CDRs are as follows: CDRH1 is located at positions 31-35 of the respective V_{H} sequence; CDRH2 is located at positions 50-66 of the respective V_{H} sequence; CDRH3 is located at positions 99-106 of the respective V_{H} sequence; CDRL1 is located at positions 24-34 of the respective V_{L} sequence; CDRL2 is location at positions 50-56 of the respective V_{L} sequence; and CDRL3 is located at positions 89-97 of the respective V_{L} sequence. BTLA binding agents having the pairing of heavy and light chain variable regions set forth in Table 2, or at least the CDRs thereof, provide specific embodiments of the disclosure. Additional pairings of the heavy and light chain variable regions of Table 2, or at least the CDRs thereof, would provide still other BTLA binding agents, and are contemplated as being within the scope of the disclosure.

Table 3 provides Examples of BTLA binding agents (e.g., antibodies or antibody fragments) comprising immunoglobulin heavy chain variable regions of SEQ ID NOs: 43-156 and light chain variable regions of SEQ ID NOs: 157-192.

TABLE 3

| | Heavy Chain | Light Chain |
|---|---|---|
| Ab Identification | Variable Region | Variable Region |
| APE08876 | SEQ ID NO: 45 | SEQ ID NO: 159 |
| APE08894 | SEQ ID NO: 45 | SEQ ID NO: 158 |
| APE08895 | SEQ ID NO: 45 | SEQ ID NO: 157 |
| APE08896 | SEQ ID NO: 44 | SEQ ID NO: 160 |
| APE08897 | SEQ ID NO: 44 | SEQ ID NO: 159 |
| APE08898 | SEQ ID NO: 44 | SEQ ID NO: 158 |
| APE08899 | SEQ ID NO: 44 | SEQ ID NO: 157 |
| APE08900 | SEQ ID NO: 43 | SEQ ID NO: 160 |
| APE08901 | SEQ ID NO: 43 | SEQ ID NO: 159 |
| APE08902 | SEQ ID NO: 43 | SEQ ID NO: 158 |
| APE08903 | SEQ ID NO: 43 | SEQ ID NO: 157 |
| APE08904 | SEQ ID NO: 45 | SEQ ID NO: 160 |
| APE09035 | SEQ ID NO: 49 | SEQ ID NO: 160 |
| APE09036 | SEQ ID NO: 48 | SEQ ID NO: 160 |
| APE09037 | SEQ ID NO: 47 | SEQ ID NO: 160 |
| APE09038 | SEQ ID NO: 46 | SEQ ID NO: 160 |
| APE09043 | SEQ ID NO: 45 | SEQ ID NO: 166 |
| APE09044 | SEQ ID NO: 45 | SEQ ID NO: 165 |
| APE09045 | SEQ ID NO: 45 | SEQ ID NO: 164 |
| APE09046 | SEQ ID NO: 45 | SEQ ID NO: 163 |
| APE09047 | SEQ ID NO: 45 | SEQ ID NO: 162 |
| APE09048 | SEQ ID NO: 45 | SEQ ID NO: 161 |
| APE09076 | SEQ ID NO: 45 | SEQ ID NO: 167 |
| APE09233 | SEQ ID NO: 44 | SEQ ID NO: 167 |
| APE09234 | SEQ ID NO: 44 | SEQ ID NO: 165 |
| APE09235 | SEQ ID NO: 44 | SEQ ID NO: 161 |
| APE09236 | SEQ ID NO: 52 | SEQ ID NO: 161 |
| APE09237 | SEQ ID NO: 51 | SEQ ID NO: 161 |
| APE09238 | SEQ ID NO: 50 | SEQ ID NO: 161 |
| APE09261 | SEQ ID NO: 52 | SEQ ID NO: 160 |
| APE09262 | SEQ ID NO: 51 | SEQ ID NO: 160 |
| APE09263 | SEQ ID NO: 50 | SEQ ID NO: 160 |
| APE09783 | SEQ ID NO: 54 | SEQ ID NO: 160 |
| APE09784 | SEQ ID NO: 53 | SEQ ID NO: 160 |
| APE09987 | SEQ ID NO: 44 | SEQ ID NO: 168 |
| APE09988 | SEQ ID NO: 45 | SEQ ID NO: 168 |
| APE10018 | SEQ ID NO: 61 | SEQ ID NO: 160 |
| APE10019 | SEQ ID NO: 60 | SEQ ID NO: 160 |
| APE10020 | SEQ ID NO: 59 | SEQ ID NO: 160 |
| APE10021 | SEQ ID NO: 58 | SEQ ID NO: 160 |
| APE10022 | SEQ ID NO: 57 | SEQ ID NO: 160 |
| APE10023 | SEQ ID NO: 56 | SEQ ID NO: 160 |
| APE10024 | SEQ ID NO: 55 | SEQ ID NO: 160 |
| APE10068 | SEQ ID NO: 65 | SEQ ID NO: 168 |
| APE10070 | SEQ ID NO: 63 | SEQ ID NO: 168 |
| APE10070 | SEQ ID NO: 64 | SEQ ID NO: 168 |
| APE10071 | SEQ ID NO: 62 | SEQ ID NO: 168 |
| APE10072 | SEQ ID NO: 65 | SEQ ID NO: 160 |
| APE10074 | SEQ ID NO: 64 | SEQ ID NO: 160 |
| APE10074 | SEQ ID NO: 63 | SEQ ID NO: 160 |
| APE10075 | SEQ ID NO: 62 | SEQ ID NO: 160 |
| APE10265 | SEQ ID NO: 83 | SEQ ID NO: 168 |
| APE10266 | SEQ ID NO: 82 | SEQ ID NO: 168 |
| APE10267 | SEQ ID NO: 81 | SEQ ID NO: 168 |
| APE10268 | SEQ ID NO: 80 | SEQ ID NO: 168 |
| APE10269 | SEQ ID NO: 79 | SEQ ID NO: 168 |
| APE10270 | SEQ ID NO: 78 | SEQ ID NO: 168 |
| APE10271 | SEQ ID NO: 77 | SEQ ID NO: 168 |
| APE10272 | SEQ ID NO: 76 | SEQ ID NO: 168 |
| APE10273 | SEQ ID NO: 75 | SEQ ID NO: 168 |
| APE10274 | SEQ ID NO: 74 | SEQ ID NO: 168 |
| APE10275 | SEQ ID NO: 73 | SEQ ID NO: 168 |
| APE10276 | SEQ ID NO: 72 | SEQ ID NO: 168 |
| APE10277 | SEQ ID NO: 71 | SEQ ID NO: 168 |
| APE10278 | SEQ ID NO: 70 | SEQ ID NO: 168 |
| APE10279 | SEQ ID NO: 69 | SEQ ID NO: 168 |
| APE10280 | SEQ ID NO: 68 | SEQ ID NO: 160 |
| APE10281 | SEQ ID NO: 67 | SEQ ID NO: 160 |
| APE10282*† | SEQ ID NO: 66 | SEQ ID NO: 160 |
| APE10284 | SEQ ID NO: 68 | SEQ ID NO: 168 |
| APE10285 | SEQ ID NO: 67 | SEQ ID NO: 168 |
| APE10286*† | SEQ ID NO: 66 | SEQ ID NO: 168 |
| APE10297 | SEQ ID NO: 99 | SEQ ID NO: 168 |
| APE10298 | SEQ ID NO: 98 | SEQ ID NO: 168 |
| APE10299 | SEQ ID NO: 97 | SEQ ID NO: 168 |
| APE10300 | SEQ ID NO: 96 | SEQ ID NO: 168 |
| APE10301 | SEQ ID NO: 95 | SEQ ID NO: 168 |
| APE10302 | SEQ ID NO: 94 | SEQ ID NO: 168 |
| APE10303 | SEQ ID NO: 93 | SEQ ID NO: 168 |
| APE10304 | SEQ ID NO: 92 | SEQ ID NO: 168 |
| APE10305 | SEQ ID NO: 91 | SEQ ID NO: 168 |
| APE10306 | SEQ ID NO: 90 | SEQ ID NO: 168 |
| APE10307 | SEQ ID NO: 89 | SEQ ID NO: 168 |
| APE10308 | SEQ ID NO: 88 | SEQ ID NO: 168 |
| APE10309 | SEQ ID NO: 87 | SEQ ID NO: 168 |
| APE10310 | SEQ ID NO: 86 | SEQ ID NO: 168 |
| APE10311 | SEQ ID NO: 85 | SEQ ID NO: 168 |
| APE10312 | SEQ ID NO: 84 | SEQ ID NO: 168 |
| APE10343 | SEQ ID NO: 65 | SEQ ID NO: 179 |
| APE10344 | SEQ ID NO: 65 | SEQ ID NO: 178 |
| APE10345 | SEQ ID NO: 65 | SEQ ID NO: 177 |
| APE10346 | SEQ ID NO: 65 | SEQ ID NO: 176 |
| APE10347 | SEQ ID NO: 65 | SEQ ID NO: 175 |
| APE10348 | SEQ ID NO: 65 | SEQ ID NO: 174 |
| APE10349 | SEQ ID NO: 65 | SEQ ID NO: 173 |
| APE10350 | SEQ ID NO: 65 | SEQ ID NO: 172 |
| APE10351 | SEQ ID NO: 65 | SEQ ID NO: 171 |
| APE10352 | SEQ ID NO: 65 | SEQ ID NO: 170 |
| APE10353 | SEQ ID NO: 65 | SEQ ID NO: 169 |
| APE10426 | SEQ ID NO: 68 | SEQ ID NO: 187 |
| APE10427 | SEQ ID NO: 68 | SEQ ID NO: 186 |
| APE10428 | SEQ ID NO: 68 | SEQ ID NO: 185 |
| APE10429 | SEQ ID NO: 68 | SEQ ID NO: 184 |
| APE10430 | SEQ ID NO: 68 | SEQ ID NO: 183 |
| APE10431 | SEQ ID NO: 68 | SEQ ID NO: 182 |
| APE10432 | SEQ ID NO: 68 | SEQ ID NO: 181 |
| APE10433 | SEQ ID NO: 68 | SEQ ID NO: 180 |
| APE10462 | SEQ ID NO: 118 | SEQ ID NO: 175 |
| APE10463 | SEQ ID NO: 117 | SEQ ID NO: 175 |
| APE10464 | SEQ ID NO: 116 | SEQ ID NO: 175 |
| APE10465 | SEQ ID NO: 115 | SEQ ID NO: 175 |
| APE10466 | SEQ ID NO: 114 | SEQ ID NO: 175 |
| APE10467 | SEQ ID NO: 113 | SEQ ID NO: 175 |
| APE10468 | SEQ ID NO: 112 | SEQ ID NO: 175 |
| APE10469 | SEQ ID NO: 111 | SEQ ID NO: 175 |
| APE10470 | SEQ ID NO: 139 | SEQ ID NO: 175 |
| APE10471 | SEQ ID NO: 138 | SEQ ID NO: 175 |
| APE10472 | SEQ ID NO: 137 | SEQ ID NO: 175 |
| APE10473 | SEQ ID NO: 136 | SEQ ID NO: 175 |
| APE10474 | SEQ ID NO: 135 | SEQ ID NO: 175 |
| APE10475 | SEQ ID NO: 134 | SEQ ID NO: 175 |
| APE10476 | SEQ ID NO: 133 | SEQ ID NO: 175 |
| APE10477 | SEQ ID NO: 132 | SEQ ID NO: 175 |
| APE10478 | SEQ ID NO: 131 | SEQ ID NO: 175 |
| APE10479 | SEQ ID NO: 130 | SEQ ID NO: 175 |
| APE10480 | SEQ ID NO: 129 | SEQ ID NO: 175 |
| APE10481 | SEQ ID NO: 128 | SEQ ID NO: 175 |
| APE10482 | SEQ ID NO: 127 | SEQ ID NO: 175 |
| APE10483 | SEQ ID NO: 126 | SEQ ID NO: 175 |
| APE10484 | SEQ ID NO: 125 | SEQ ID NO: 175 |
| APE10485 | SEQ ID NO: 124 | SEQ ID NO: 175 |
| APE10486 | SEQ ID NO: 123 | SEQ ID NO: 175 |
| APE10487 | SEQ ID NO: 122 | SEQ ID NO: 175 |
| APE10488 | SEQ ID NO: 121 | SEQ ID NO: 175 |
| APE10489 | SEQ ID NO: 120 | SEQ ID NO: 175 |
| APE10490 | SEQ ID NO: 119 | SEQ ID NO: 175 |
| APE10491 | SEQ ID NO: 110 | SEQ ID NO: 175 |
| APE10492 | SEQ ID NO: 109 | SEQ ID NO: 175 |
| APE10493 | SEQ ID NO: 108 | SEQ ID NO: 175 |
| APE10494 | SEQ ID NO: 107 | SEQ ID NO: 175 |
| APE10495 | SEQ ID NO: 106 | SEQ ID NO: 175 |
| APE10496 | SEQ ID NO: 105 | SEQ ID NO: 175 |
| APE10497 | SEQ ID NO: 104 | SEQ ID NO: 175 |
| APE10498 | SEQ ID NO: 103 | SEQ ID NO: 175 |
| APE10499 | SEQ ID NO: 102 | SEQ ID NO: 175 |
| APE10500 | SEQ ID NO: 101 | SEQ ID NO: 175 |
| APE10501 | SEQ ID NO: 100 | SEQ ID NO: 175 |
| APE10513 | SEQ ID NO: 139 | SEQ ID NO: 174 |
| APE10514 | SEQ ID NO: 138 | SEQ ID NO: 174 |
| APE10515 | SEQ ID NO: 137 | SEQ ID NO: 174 |
| APE10516 | SEQ ID NO: 136 | SEQ ID NO: 174 |
| APE10517 | SEQ ID NO: 135 | SEQ ID NO: 174 |
| APE10518 | SEQ ID NO: 134 | SEQ ID NO: 174 |
| APE10519 | SEQ ID NO: 133 | SEQ ID NO: 174 |
| APE10520 | SEQ ID NO: 132 | SEQ ID NO: 174 |
| APE10521 | SEQ ID NO: 131 | SEQ ID NO: 174 |
| APE10522 | SEQ ID NO: 130 | SEQ ID NO: 174 |
| APE10523 | SEQ ID NO: 129 | SEQ ID NO: 174 |
| APE10524 | SEQ ID NO: 128 | SEQ ID NO: 174 |
| APE10525 | SEQ ID NO: 127 | SEQ ID NO: 174 |
| APE10526 | SEQ ID NO: 126 | SEQ ID NO: 174 |
| APE10527 | SEQ ID NO: 125 | SEQ ID NO: 174 |
| APE10528 | SEQ ID NO: 124 | SEQ ID NO: 174 |
| APE10529 | SEQ ID NO: 123 | SEQ ID NO: 174 |
| APE10530 | SEQ ID NO: 122 | SEQ ID NO: 174 |
| APE10531 | SEQ ID NO: 121 | SEQ ID NO: 174 |
| APE10532 | SEQ ID NO: 120 | SEQ ID NO: 174 |
| APE10533 | SEQ ID NO: 119 | SEQ ID NO: 174 |
| APE10534 | SEQ ID NO: 110 | SEQ ID NO: 174 |
| APE10535 | SEQ ID NO: 109 | SEQ ID NO: 174 |
| APE10536 | SEQ ID NO: 108 | SEQ ID NO: 174 |
| APE10537 | SEQ ID NO: 107 | SEQ ID NO: 174 |
| APE10538 | SEQ ID NO: 106 | SEQ ID NO: 174 |
| APE10539 | SEQ ID NO: 105 | SEQ ID NO: 174 |
| APE10540 | SEQ ID NO: 104 | SEQ ID NO: 174 |
| APE10541 | SEQ ID NO: 103 | SEQ ID NO: 174 |
| APE10542 | SEQ ID NO: 102 | SEQ ID NO: 174 |
| APE10543 | SEQ ID NO: 101 | SEQ ID NO: 174 |
| APE10544 | SEQ ID NO: 100 | SEQ ID NO: 174 |
| APE10545 | SEQ ID NO: 118 | SEQ ID NO: 174 |
| APE10546 | SEQ ID NO: 117 | SEQ ID NO: 174 |
| APE10547 | SEQ ID NO: 116 | SEQ ID NO: 174 |
| APE10548 | SEQ ID NO: 115 | SEQ ID NO: 174 |
| APE10549 | SEQ ID NO: 114 | SEQ ID NO: 174 |
| APE10550 | SEQ ID NO: 113 | SEQ ID NO: 174 |
| APE10551 | SEQ ID NO: 112 | SEQ ID NO: 174 |
| APE10552 | SEQ ID NO: 111 | SEQ ID NO: 174 |
| APE10553 | SEQ ID NO: 68 | SEQ ID NO: 189 |
| APE10554 | SEQ ID NO: 68 | SEQ ID NO: 188 |
| APE10555 | SEQ ID NO: 143 | SEQ ID NO: 174 |
| APE10556 | SEQ ID NO: 142 | SEQ ID NO: 174 |
| APE10570† | SEQ ID NO: 141 | SEQ ID NO: 174 |
| APE10571 | SEQ ID NO: 140 | SEQ ID NO: 174 |
| APE10577 | SEQ ID NO: 68 | SEQ ID NO: 174 |
| APE10580 | SEQ ID NO: 146 | SEQ ID NO: 189 |
| APE10581 | SEQ ID NO: 145 | SEQ ID NO: 189 |
| APE10583 | SEQ ID NO: 146 | SEQ ID NO: 174 |
| APE10584 | SEQ ID NO: 145 | SEQ ID NO: 174 |
| APE10620 | SEQ ID NO: 149 | SEQ ID NO: 189 |
| APE10621 | SEQ ID NO: 148 | SEQ ID NO: 189 |
| APE10622 | SEQ ID NO: 147 | SEQ ID NO: 189 |
| APE10623 | SEQ ID NO: 149 | SEQ ID NO: 174 |
| APE10624 | SEQ ID NO: 148 | SEQ ID NO: 174 |
| APE10625 | SEQ ID NO: 147 | SEQ ID NO: 174 |
| APE10657† | SEQ ID NO: 153 | SEQ ID NO: 174 |
| APE10658† | SEQ ID NO: 152 | SEQ ID NO: 174 |
| APE10659 | SEQ ID NO: 151 | SEQ ID NO: 174 |
| APE10660 | SEQ ID NO: 118 | SEQ ID NO: 191 |
| APE10661 | SEQ ID NO: 147 | SEQ ID NO: 190 |
| APE10663 | SEQ ID NO: 118 | SEQ ID NO: 190 |
| APE10664† | SEQ ID NO: 150 | SEQ ID NO: 189 |
| APE10665† | SEQ ID NO: 150 | SEQ ID NO: 174 |
| APE10677† | SEQ ID NO: 153 | SEQ ID NO: 192 |
| APE10679 | SEQ ID NO: 151 | SEQ ID NO: 192 |
| APE10678† | SEQ ID NO: 152 | SEQ ID NO: 192 |
| APE10680† | SEQ ID NO: 156 | SEQ ID NO: 174 |
| APE10681† | SEQ ID NO: 155 | SEQ ID NO: 174 |
| APE10682 | SEQ ID NO: 154 | SEQ ID NO: 174 |
| APE10840 | SEQ ID NO: 144 | SEQ ID NO: 174 |

For the antibodies or antibody fragments in Table 3, the Kabat numbered CDR regions are as follows: CDRH1 is located at positions 31-35 of the respective V_{H} sequence; CDRH2 is located at positions 50-66 of the respective V_{H} sequence, except for the antibodies marked with *, in which CDRH2 is located a positions 50-67 of the respective V_{H} sequence; CDRH3 is located at positions 99-113 of the respective V_{H} sequence, except for the antibodies marked with ∀, in which CDRH3 is located at positions 100-114; CDRL1 is located at positions 24-34 of the respective V_{L} sequence; CDRL2 is location at positions 50-56 of the respective V_{L} sequence; CDRL3 is located at positions 89-97 of the respective V_{L} sequence. BTLA binding agents having the pairing of heavy and light chain variable regions set forth in Table 3, or at least the CDRs thereof, provide specific embodiments of the disclosure. Additional pairings of the heavy and light chain variable regions of Table 3, or at least the CDRs thereof, would provide still other BTLA binding agents, and are contemplated as being within the scope of the disclosure.

In the following examples, the antibodies referenced are as follows:

| | SEQ ID NOs | | | | | | | |
|---|---|---|---|---|---|---|---|---|
| | | | | | | | Heavy | Light |
| | | | | | | | Chain | Chain |
| | | | | | | | Variable | Variable |
| Ab No. | CDRH1 | CDRH2 | CDRH3 | CDRL1 | CDRL2 | CDRL3 | Region | Region |
| 6G3 | | | | | | | | |
| APE10840.x | 201 | 202 | 203 | 204 | 205 | 206 | 144 | 174 |
| APE12839.x | 201 | 202 | 203 | 204 | 205 | 206 | 144 | 174 |
| APE13308.x | 201 | 202 | 203 | 204 | 205 | 206 | 144 | 174 |
| APE10585.x | 201 | 202 | 203 | 204 | 205 | 206 | 144 | 174 |
| 10D8 | | | | | | | | |
| APE10134 | 211 | 212 | 213 | 214 | 215 | 216 | 207 | 209 |
| APE12774.x | 221 | 222 | 223 | 224 | 225 | 226 | 217 | 219 |
| APE11482 | 221 | 222 | 223 | 224 | 225 | 226 | 217 | 219 |

### Example 1

Antibody 6G3 was derived from a mouse hybridoma generated by standard fusion techniques from spleen cells of a BTLA immunized mouse. The antibody was humanized using standard techniques described herein. The final optimized antibody was expressed in Chinese Hamster Ovary (CHO) cells using the vectors summarized in Table 1.

TABLE 1

| | | H Chain | L Chain |
|---|---|---|---|
| | Expression | Variable | Variable |
| 6G3 | Conditions | Region | Region |
| APE10840 | ExpiCHO-S ™ | SEQ ID NO: 144 | SEQ ID NO: 174 |
| | transient | | |
| APE12839 | ExpiCHO-S sorted | SEQ ID NO: 144 | SEQ ID NO: 174 |
| | stable pool | | |
| APE13308 | CHO-K1 stable pool | SEQ ID NO: 144 | SEQ ID NO: 174 |

### Example 2

This example demonstrates that the 6G3 anti-BTLA antibody disclosed herein has a binding affinity as determined by surface plasmon resonance for human BTLA of K_{D}˜5 nM and for cynomolgus monkey BTLA of K_{D}˜11 nM.

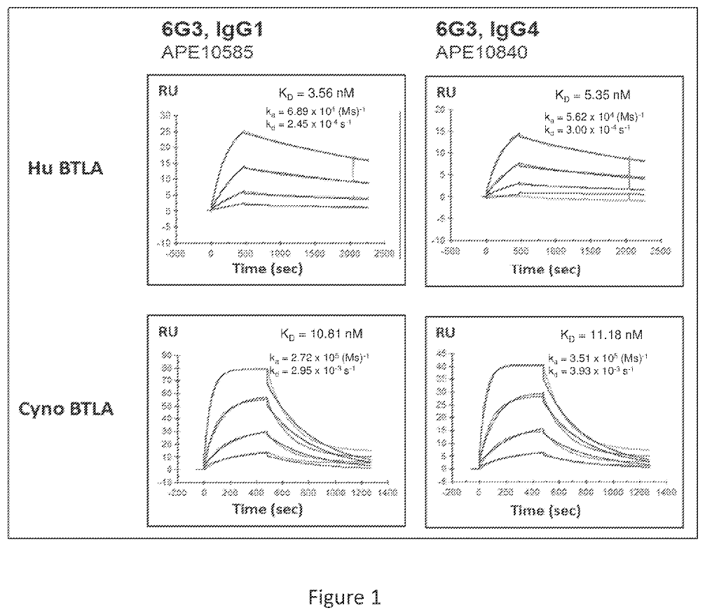

Surface plasmon resonance (SPR) analyses were carried out using a Biacore T200 (GE Healthcare Life Sciences). Kinetic constants were determined using a 1:1 binding model in the Biacore T200 Evaluation Software to calculate on- and off-rates (kₐ and k_{d}, respectively), and dissociation constants as a measure of overall affinity (K_{D}). Anti-human IgG (GE Healthcare Life Sciences) was immobilized on a CM5 chip using EDC-activated amine coupling chemistry (420 seconds contact at a flow rate of 10 μl/min). Antibodies (APE10585.04 6G3 IgG1 and APE10840.05 6G3 IgG4), each at 0.5 μg/ml) were captured in a 60 second contact with the flow cell using a flow rate of 10 μl/min. Monomeric human BTLA-his or cynomolgus monkey BTLA-his at 60 nM, 20 nM, 6.7 nM and 2.2 nM concentrations was flowed over captured antibody (480 seconds association, 1800 seconds dissociation). Runs were at 25° C. and used buffer containing 10 mM HEPES, pH 7.6, 150 mM NaCl, 3 mM EDTA, 0.05% polysorbate 20 (HBS-EP+, pH 7.6; Teknova) for the mobile phase and all dilutions. At the end of each cycle, bound antigen was removed by regeneration with two successive exposures (60 seconds and 90 seconds) to 3 M MgCl2 (30 μl/min). Sensorgrams and binding constants corresponding to the experiment are shown in FIG. 1 (human BTLA) and FIG. 1 (cynomolgus monkey BTLA). Antibody capture levels in resonance units (RU) are listed at the right side of each panel.

### Example 3

This example demonstrates that the 6G3 anti-BTLA antibody disclosed herein has a binding affinity as determined by Kinetic Exclusion Assay for human BTLA of K_{D}˜410 pM and for cynomolgus monkey BTLA of K_{D}˜1.66 nM.

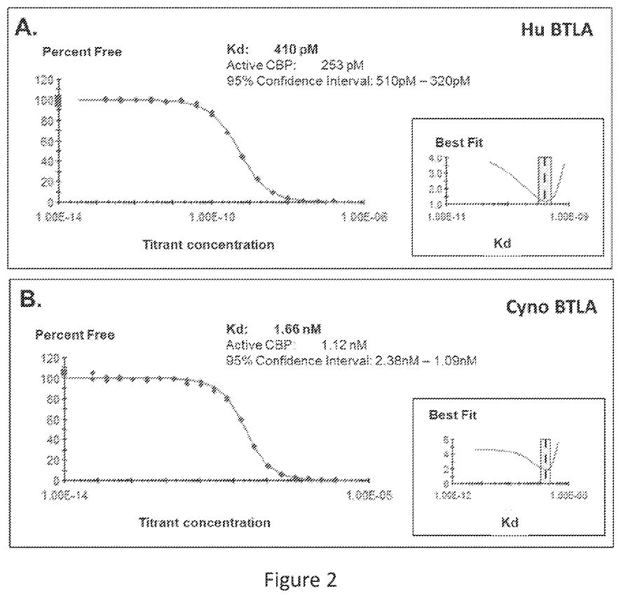

Solution-based affinity measurements of 6G3 IgG4 (APE10840.04 (H Chain SEQ ID NO: 144, L Chain SEQ ID NO: 174)) binding to BTLA were determined on a KinExA 3000 (Sapidyne Instruments). Azlactone beads (ThermoFisher Scientific) were coated with human BTLA ECD-his (30 μg/ml) or cynomolgus monkey BTLA ECD-his (20 μg/ml) in 50 mM Na₂CO₃ for 2 hours at room temperature and blocked with 10 mg/ml BSA. 6G3 IgG4 APE10840.04 concentration was kept constant at 100 pM for binding in both human and cynomolgus monkey BTLA experiments. Human or cynomolgus monkey BTLA ECD-his was added at 25° C. in 2.5-fold dilutions from 1000 nM to 68 fM. Sample sets were equilibrated at 4° C. for 72 hours and brought to room temperature for 6 hours before capturing free antibody with BTLA-coupled Azlactone beads. Secondary antibody for quantifying 6G3 bound to beads was Alexa-Fluor-647 AffiPure Donkey-anti-human IgG (250 ng/ml, Jackson ImmunoResearch Laboratories). Maximum and background signals were determined on samples with 6G3 only and buffer only, respectively. Data were analyzed using KinExA Pro Software 3.2.6. The 95% confidence intervals in the analyses of the K_{D} values for 6G3 antibody binding to human BTLA and cynomolgus monkey BTLA are shown in FIG. 2.

### Example 4

This example demonstrates that the 6G3 antibody disclosed herein exhibits saturation binding to human and cynomolgus monkey BTLA expressed in stably transfected 293c18 cells.

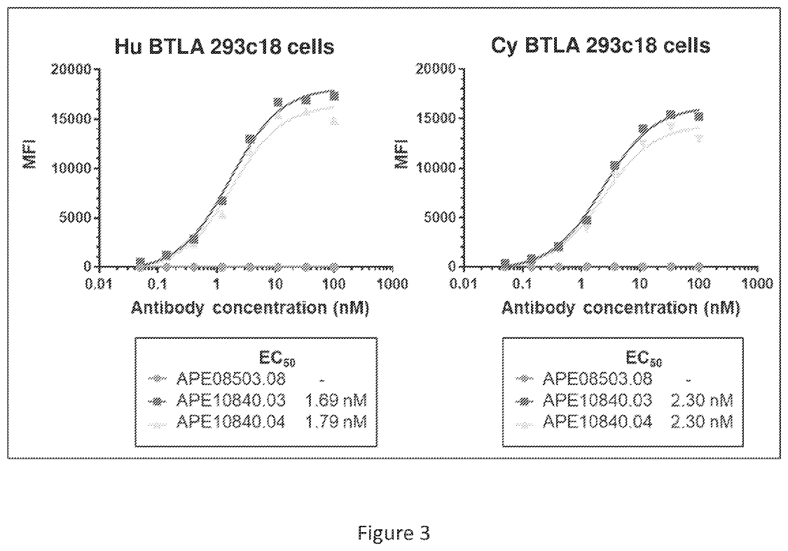

293c18 cell clone 1E4 stably expressing human BTLA construct or clone 1G5 stably expressing cynomolgus monkey BTLA were harvested with Accutase solution (Millipore Sigma/Sigma-Aldrich) and washed once with Phosphate-Buffered Saline, 1% BSA. 293c18 cynomolgus monkey BTLA cells were loaded with the lipophilic carbocyanine dye, DiD (2 μM, 1,1′-dioctadecyl-3,3,3′,3′-tetramenthylindodicarbocyanine; ThermoFisher Scientific) by rocking gently for 10 minutes at room temperature. DiD-stained cells were washed in PBS, 1% BSA and equal amounts of human BTLA 293c18 cells and cynomolgus monkey BTLA 293c18-DiD-stained cells were mixed. Cells (2×10⁵ total cells/sample) were incubated with the indicated concentrations of purified 6G3 IgG4 (two production lots, APE10840.03 and APE10840.04 (H Chain SEQ ID NO: 144, L Chain SEQ ID NO: 174)) or human IgG4 isotype control antibody specific for hen egg lysozyme with gentle shaking for 10 minutes at 4° C. in FACS buffer (PBS, 1% BSA, 0.02% sodium azide). Cells were centrifuged and washed once with FACS buffer, resuspended in FACS buffer, and incubated with gentle shaking for an additional 20 minutes at 4° C. Cells were centrifuged and washed once with FACS buffer without BSA and fixed in 100 μl/well 2% paraformaldehyde in PBS for 10 minutes at room temperature. Cells were washed once in FACS buffer and incubated with gentle shaking for 10 minutes at 4° C. with secondary antibody (Goat Anti-Human Kappa-PE, 0.2 μg/ml in FACS buffer, SouthernBiotech). Cells were washed and resuspended in FACS buffer and analyzed for fluorescence on a BD FACSArray (BD Biosciences). DiD stained cells expressing cynomolgus monkey BTLA (FIG. 3) were analyzed for median fluorescence intensity (MFI) separately from cells expressing human BTLA (FIG. 3). Curves were fit with log (inhibitor) vs. response-variable slope (four parameters) least squares fit analyses in GraphPad Prism (GraphPad Software, Inc.)

Two lots of the 6G3 antibody disclosed herein (APE10840.03 and APE10840.04) demonstrate similar concentration-dependent and saturating binding to human BTLA 293c18 cells (EC₅₀˜1.7 nM and 1.8 nM, respectively) and cynomolgus monkey BTLA 293c18 cells (EC₅₀˜2.3 nM) as shown in FIG. 3.

### Example 5

This example demonstrates that the 6G3 antibody disclosed herein exhibits saturation binding to normal donor human peripheral blood CD4⁺ T cells, CD8⁺ T cells, and CD20⁺ B cells.

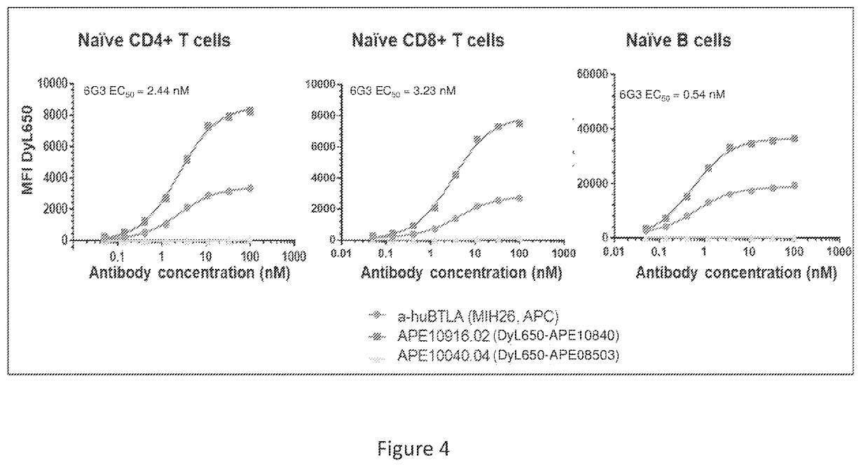

Human peripheral blood mononuclear cells (PBMCs) were isolated by Histopaque (Sigma-Aldrich) density-gradient centrifugation of normal donor blood obtained from the San Diego Blood Bank (San Diego, CA). PBMCs were washed and incubated at 2×10⁷ cells/ml in FACS buffer (PBS, 1% BSA, 0.02% sodium azide) with Human BD Fc Block (2.5 μg/1×10⁶ cells, BD Biosciences) and LIVE/DEAD® Fixable Yellow Dead Cell Stain (30 μl, ThermoFisher Scientific) for 10 minutes on ice. Cells were washed once in FACS buffer, resuspended in FACS buffer at 1×10⁷ cells/ml and the following phenotyping antibodies added: Alexa Fluor 488-anti-human CD38 clone SK7, Brilliant Violet 421-anti-human CD4 clone OKT4, Brilliant Violet 785-anti-human CD8 clone SK1, and Brilliant Violet 570-anti-human CD20 clone 2H7 (50 μl each; all from BioLegend, Inc.). Cells (1×10⁶ cells/sample) with added phenotyping antibodies were plated in a U-bottom 96-well plate and incubated with the indicated concentrations of DyLight 650-labeled anti-BTLA or isotype control antibodies with gentle shaking for 20 minutes at 4° C. in FACS buffer. 6G3 IgG4 (APE10916.02) was APE10840 labeled with DyLight 650 (3.44 mol DyL650/mol antibody). Human IgG4 isotype control antibody specific for hen egg lysozyme was labeled with DyLight 650 (3.22 mol DyL650/mol antibody). Reference anti-BTLA antibody MIH26 was purchased as an Allophycocyanin (APC)-labeled antibody from BioLegend, Inc. Samples were washed once in FACS buffer, resuspended in 150 μl/well FACS buffer and additionally washed for 10 minutes at 4° C. Samples were centrifuged and fixed in 4% paraformaldehyde in Phosphate-Buffered Saline for 10 minutes at room temperature. Samples were washed twice in FACS buffer, resuspended in 150 μl/well FACS buffer, and analyzed for fluorescence on a NovoCyte Flow Cytometer (ACEA Biosciences, Inc.). Data were analyzed using NovoExpress Software (ACEA Biosciences, Inc.). Median fluorescence intensities of anti-BTLA or isotype staining on gated CD4⁺ T cells (FIG. 4), CD8⁺ T cells (FIG. 4), or CD20⁺ B cells (FIG. 4) were graphed and curves fit with log (agonist) vs. response (three parameters) least squares fit for EC₅₀ calculation in GraphPad Prism (GraphPad Software, Inc.).

The 6G3 antibody disclosed herein labeled with DyLight 650 shows concentration-dependent and saturating binding to healthy donor CD4⁺ T cells (FIG. 4), CD8⁺ T cells (FIG. 4), and B cells (FIG. 4). The 6G3 antibody binds to CD4⁺ T cells with an EC₅₀˜2.4 nM, to CD8⁺ T cells with an EC₅₀˜3.2 nM, and to CD20⁺ B cells with an EC₅₀˜0.5 nM. Staining with positive control anti-BTLA antibody MIH26-APC parallels that of the 6G3 antibody. DyLight 650-labeled human IgG4 isotype control antibody does not show staining of these cell populations (FIG. 4).

### Example 6

This example demonstrates that the 6G3 antibody disclosed herein exhibits concentration-dependent binding to normal cynomolgus monkey peripheral blood CD3⁺ T cells and CD20⁺ B cells.

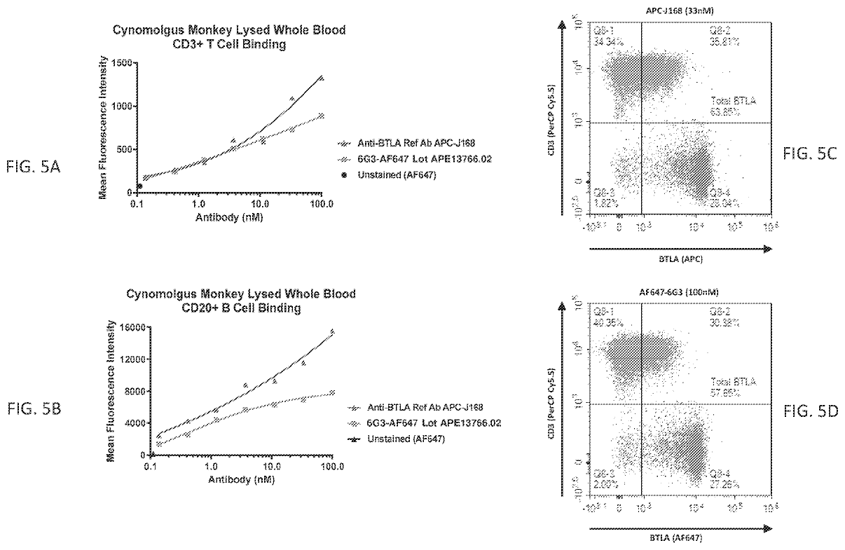

6G3 IgG4 (APE13308) manufactured from a pool of stably transfected CHO-K1 cells was labeled with Alexa Fluor 647 (AF647) (Alexa Fluor Antibody Labeling Kit; ThermoFisher Scientific/Molecular Probes) according to the manufacturer's instructions and designated APE13766.02 (6G3-AF647). Fresh peripheral blood from normal cynomolgus monkeys was obtained from Altasciences. A whole blood sample (800 μl) was incubated for 10 minutes at room temperature with FcR Blocking Reagent, human (Miltenyi Biotec, Inc.) and then stained with a mixture of fluorescently labeled antibodies to distinguish cynomolgus monkey T and B cell populations [PerCP-Cy5.5 Mouse Anti-Human CD3 (clone SP34-2; BD Biosciences), BD Horizon V450 Mouse Anti-Human CD4 (clone L200; BD Biosciences), APC/Cy7 Mouse Anti-Human CD8 (clone SK1; BD Biosciences), Brilliant Violet 785 anti-CD20 (clone 2H7; BioLegend, Inc.), BD Horizon V500 Mouse Anti-NHP CD45 (clone D058-1283; BD Biosciences)] at room temperature in the dark for 20 minutes. Blood was then aliquoted into wells and incubated with the indicated concentrations of 6G3-AF647 (APE13766.02), or APC-Mouse Anti-Human CD272 (BTLA) reference antibody (clone J168-540; BD Biosciences) for 30 minutes at room temperature, in the dark. Following incubation, red blood cells were lysed for 10 minutes by addition of 2.0 ml diluted BD Pharm Lyse lysing solution (BD Biosciences), samples centrifuged at 200×g for 5 minutes, washed in FACS Buffer [Dulbecco's PBS, no calcium, no magnesium (Gibco/ThermoFisher Scientific), 25 mM HEPES, pH 7.2, 0.1% BSA, 0.1% sodium azide] and fixed in 4% paraformaldehyde (200 μl/sample) for 10 minutes at room temperature. Samples were washed twice in FACS Buffer and analyzed for fluorescence on a NovoCyte Quanteon flow cytometer (ACEA Biosciences, Inc.). Data were analyzed using NovoExpress Software (ACEA Biosciences, Inc.). Mean fluorescence intensity (MFI) values were graphed and fit with nonlinear regression analyses in GraphPad Prism (GraphPad Software, Inc.). MFI values for each anti-BTLA antibody on total CD3⁺ cells are shown in FIG. 5A; MFI values for each anti-BTLA antibody on total CD20⁻ cells are shown in FIG. 5B. FIG. 5C shows a dot plot analysis of anti-CD3 staining and 33 nM APC-labeled anti-BTLA reference antibody (clone J168-540) staining. FIG. 5D shows a dot plot analysis of anti-CD3 staining and 100 nM 6G3-AF647 staining. Binding of each anti-BTLA antibody to CD3⁻ cells in FIG. 5C and FIG. 5D (green cells in the bottom quadrants of each panel) reflects the binding to CD20+ B cells.

The 6G3 antibody disclosed herein labeled with AF647 shows concentration-dependent binding to peripheral blood CD3⁺ T cells and CD20⁺ B cells from a normal cynomolgus monkey. The binding to CD3⁺ peripheral blood T cells does not saturate at 100 nM antibody under the staining conditions used (FIG. 5A) (estimated EC₅₀˜3.4 nM), and the binding of 6G3-AF647 to CD20⁺ peripheral blood B cells is starting to saturate (approximate EC₅₀˜1.4 nM) (FIG. 5B). The MFI of 6G3-AF647 staining of B cells is approximately 9-fold greater than the MFI of 6G3-AF647 staining of T cells. The staining pattern of 6G3-AF647 at 100 nM on normal CD3⁺ and CD3⁻ (CD20⁺) cynomolgus monkey cells closely resembles the staining pattern of the commercially available reference APC-labeled anti-BTLA antibody J168 at 33 nM (FIGS. 5D and 5C, respectively).

### Example 7

This example demonstrates that the anti-BTLA antibody disclosed herein does not compete with HVEM or HVEM/LIGHT complex for binding to cell surface BTLA.

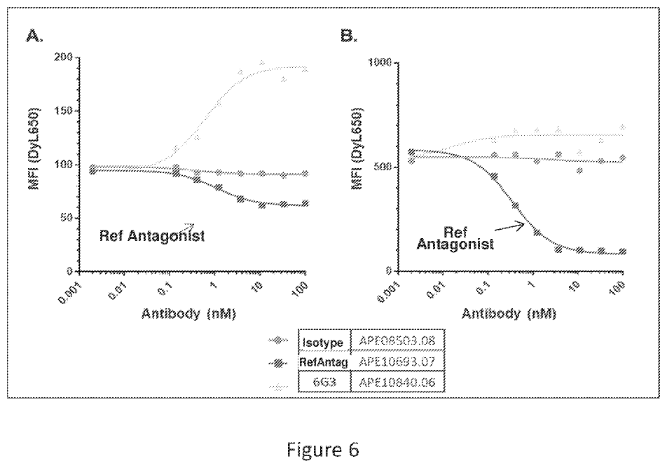

293c18 cells, clone 1E4 stably expressing human BTLA were harvested with Accutase solution (Millipore Sigma/Sigma-Aldrich) and washed once with PBS, 1% BSA. Cells were plated at 2×10⁵/well in U-bottom 96-well plates and placed on ice. Purified antibodies at twice the indicated concentrations were serially diluted 3-fold in FACS buffer (PBS, 1% BSA, 0.02% sodium azide). Antibodies tested were human IgG4 isotype control antibody specific for hen egg lysozyme, a reference BTLA IgG4 antagonist antibody, and 6G3 IgG4 (APE10840.06). Trimeric HVEM/LIGHT complexes were pre-formed by mixing equimolar (60 nM each) amounts of DyLight 650-HVEM human IgG1 Fc and trimeric LIGHT-foldon-his and pre-incubating for 15 minutes at room temperature. Either DyLight 650-HVEM-Fc (final concentration 100 nM; FIG. 6A) or pre-formed DyLight 650-HVEM/LIGHT complexes (final concentration of HVEM/LIGHT, 30 nM each; FIG. 6B) were added to the diluted antibodies (final concentrations of antibodies as indicated) and incubated for 15 minutes on ice. Cells were centrifuged, resuspended gently in the mixture of antibodies and HVEM (FIG. 6A) or antibodies and HVEM/LIGHT complexes (FIG. 6B) and incubated for 30 minutes on ice. Cells were centrifuged, washed once with FACS buffer, and fixed in 2% paraformaldehyde in PBS for 10 minutes at room temperature. Cells were washed once, resuspended in FACS buffer and analyzed for fluorescence on a BD FACSArray (BD Biosciences). Median fluorescence intensities (MFI) were graphed in GraphPad Prism (GraphPad Software, Inc.) and curves fit with log (agonist) vs. response (three parameters) least squares fit.

The 6G3 antibody disclosed herein (APE10840.06) does not compete with either the binding of HVEM-Fc (FIG. 6A) or the binding of the HVEM/LIGHT complex (FIG. 6B) to cell surface BTLA. In the presence of the 6G3 antibody, increased binding of HVEM-Fc to cell surface BTLA is observed (FIG. 6A). The 6G3-dependent increased HVEM binding is also observed when HVEM/LIGHT complexes were used but is more pronounced with HVEM-Fc alone (FIGS. 6A and 6B). A reference antagonist antibody shows a concentration-dependent inhibition of HVEM and HVEM/LIGHT binding to BTLA (FIGS. 6A and 6B). An irrelevant isotype-matched IgG4 antibody does not affect binding of HVEM or HVEM/LIGHT to BTLA.

### Example 8

This example demonstrates that the epitope on human BTLA that is bound by the 6G3 and 10D8 antibodies disclosed herein is on the opposite face of BTLA from the HVEM binding site.

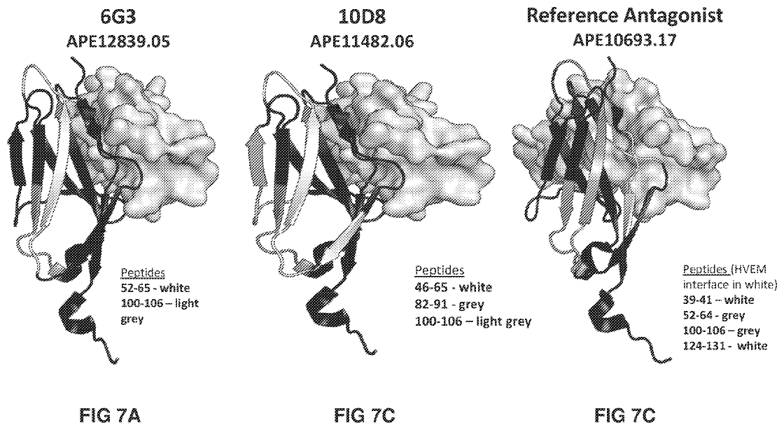

Hydrogen-deuterium exchange mapping of the peptides on BTLA bound by the 6G3 and 10D8 antibodies disclosed herein was performed using recombinant human BTLA monomer. The BTLA used in the experiment was hBTLA amino acid numbers 31-155 (UniProt ID #Q7Z6A9) followed by a 6-his tag. FIGS. 7A, 7B, and 7C show a ribbon model of the crystal structure of human BTLA extracellular domain (black) in complex with a space-filling model of the crystal structure of human HVEM extracellular binding domain (light gray) rendered in PyMOL from Protein Data Bank structure (Compaan et al., J. Biol Chem 280: 39553-39561 (2005)). FIG. 7A summarizes a hydrogen-deuterium exchange experiment using the 6G3 antibody (APE12839.05 (H Chain SEQ ID NO: 144, L Chain SEQ ID NO: 174)), FIG. 7B summarizes a hydrogen-deuterium exchange experiment using the 10D8 antibody (APE11482.06), and FIG. 7C summarizes a hydrogen-deuterium exchange experiment using a reference BTLA antagonist antibody (APE10693.17). For each experiment, the BTLA/antibody mixture or BTLA alone was deuterium labeled for 4, 10, or 60 minutes in order to see the exchange kinetics. After incubation, the BTLA protein was subjected to rapid enzymatic proteolysis at acidic pH and the incorporation of deuterium into resulting peptides quantified by liquid chromatography-mass spectrometry.

Mapping showed involvement of the following residues of BLTA:

6G3 Binding
Peptide 52-65
(SEQ ID NO: 227)
DPFELECPVKYCAN
Peptide 100-106
(SEQ ID NO: 228)
LHFEPVL
10D8 Binding
Peptide 46-65
(SEQ ID NO: 229)
HSILAGDPFELECPVKYCAN
Peptide 82-91
(SEQ ID NO: 230)
LEDRQTSWKE
Peptide 100-106
(SEQ ID NO: 231)
LHFEPVL
Reference Antagonist Binding
Peptide 39-41
(SEQ ID NO: 232)
YIK
Peptide 52-64
(SEQ ID NO: 233)
DPFELECPVKYCA
Peptide 100-106
(SEQ ID NO: 228)
LHFEPVL
Peptide 124-131
(SEQ ID NO: 234)
IESHSTTL

### Example 9

This example demonstrates that when BTLA and HVEM are expressed on the same cell, the 6G3 antibody disclosed herein inhibits LIGHT-induced HVEM signaling in an NF-κB luciferase reporter assay.

293c18 cells stably expressing full-length human BTLA, full-length human HVEM, and an NF-κB-luciferase reporter construct derived from pGL4.32 [luc2P/NF-κB-RE/Hygro] (Promega) were generated, single cell cloned, and designated huHVEM/huBTLA/NF-κB luciferase, clone 8. CHO-S cells stably expressing full-length human LIGHT were generated and sorted twice for the highest LIGHT expression as a stable pool. huHVEM/huBTLA/NF-κB luciferase cells were harvested with Accutase solution (Sigma-Aldrich/Millipore Sigma), resuspended in Dulbecco's Modified Eagle's Medium supplemented with 10% fetal bovine serum, and plated (5×10⁴ cells/well) in a flat-bottom 96-well plate. After 1-hour incubation at 37° C., 5% CO₂, the indicated concentrations of 6G3 IgG4 (APE12839.07 (H Chain SEQ ID NO: 144, L Chain SEQ ID NO: 174)), human IgG4 isotype control antibody specific for hen egg lysozyme, or human IgG4 reference anti-BTLA antagonist antibody were added to the cells. After a 30-minute incubation at room temperature, CHO-S LIGHT cells were harvested and added (0.55×10⁴ cells/well; final conditions, 9:1 HVEM-NF-κB responder: CHO-S LIGHT stimulator cells, which represented the EC₅₀ of the NF-κB response). After 4 hours at 37° C., 5% CO₂, an equal volume of Steady-Glo Luciferase Assay System (Promega) was added to the wells and allowed to incubate at room temperature for 10 minutes. Luminescence of samples was read on an En Vision Multimode Plater Reader (PerkinElmer) with a measurement time of 0.1 seconds. Luminescence in relative light units (RLU) was graphed and curves fit with a log (agonist) vs. response (three parameters) least squares fit in GraphPad Prism (GraphPad Software).

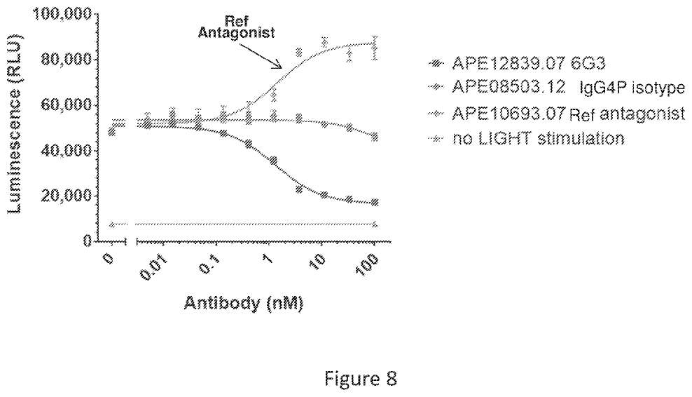

The NF-κB luciferase reporter assay shown in FIG. 8 demonstrates that the 6G3 antibody disclosed herein inhibits HVEM-dependent NF-κB signaling in a concentration-dependent manner in response to CHO-S LIGHT when BTLA and HVEM are expressed on the same cell. This suggests that under these conditions the 6G3 antibody disclosed herein may promote the interaction between HVEM and BTLA on the same cell, resulting in reduced LIGHT-driven HVEM-dependent NF-κB signaling.

The NF-κB luciferase reporter assay shown in FIG. 8 demonstrates that the reference antagonist antibody enhances LIGHT-mediated HVEM-dependent NF-κB signaling in a concentration-dependent manner in response to CHO-S LIGHT when BTLA and HVEM are expressed on the same cell. By disrupting the interaction of BTLA and HVEM on the same cell the antagonist antibody would allow more unbound HVEM molecules to be available to interact with LIGHT, thereby increasing LIGHT-driven HVEM-dependent NF-κB signaling.

### Example 10

This example demonstrates in a fluorescence resonance energy transfer (FRET) assay that the 6G3 antibody disclosed herein does not disrupt the BTLA/HVEM complex on the same cell surface.

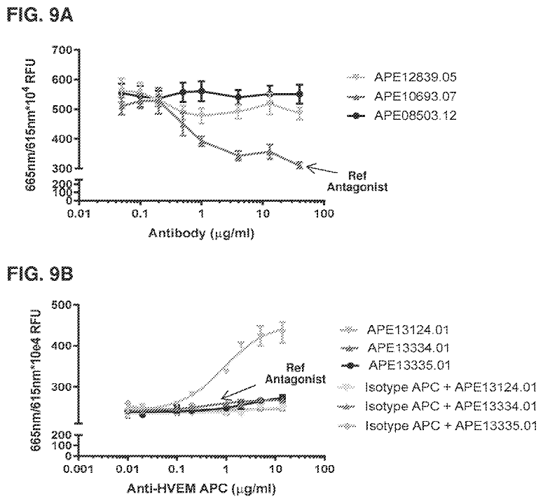

In FIG. 9A, a 293c18 cell clone stably expressing full length human BTLA and human HVEM, was harvested with Accutase solution (Millipore Sigma/Sigma-Aldrich), washed in FACS buffer (PBS, 1% BSA, 0.02% sodium azide), and plated in white U-bottom 96-well plates (2×10⁵ cells/well). Cells were centrifuged, antibodies serially diluted 3-fold at the indicated concentrations were added, and cells were incubated for 1 hour with gentle shaking at 4° C. Antibodies tested were 6G3 IgG4 (APE12839.05 (H Chain SEQ ID NO: 144, L Chain SEQ ID NO: 174)), reference antagonist antibody IgG4, and a human IgG4P isotype control antibody specific for hen egg lysozyme. Cells were centrifuged, washed in FACS buffer, and resuspended in the FRET acceptor antibody, APC-anti-HVEM (15 μg/ml; clone 122, BioLegend). The FRET donor antibody was biotin-humanized 10D8 (APE12774.02) and was pre-complexed at 10 μg/ml with 0.025 μg/ml Streptavidin-Eu (LANCE Eu-W8044-Streptavidin AD0060, PerkinElmer) by incubating for 20 minutes at room temperature. The donor complex biotin-anti-BTLA/Streptavidin-Eu was added to the plate with the acceptor APC-anti-HVEM antibody and incubated 24 hours at 4° C. Plates were washed in cold FACS buffer and fluorescence of samples was read on an EnVision Multimode Plater Reader (PerkinElmer, Santa Clara, CA). The ratio of fluorescence at 665 nm/615 nm was graphed in GraphPad Prism (GraphPad Software). Each point is the mean±SEM of three independent replicate experiments. Each sample condition for each experiment was in 5 replicate wells.

The FRET assay shown in FIG. 9A demonstrates the existence of a BTLA/HVEM complex on the transfected 293c18 cells and that the 6G3 antibody disclosed herein shows no inhibition of the BTLA/HVEM FRET signal at concentrations up to 40 μg/ml, similar to the isotype control antibody. This result confirms that the 6G3 antibody disclosed herein does not inhibit the interaction of BTLA and HVEM on the same cell surface.

The FRET assay shown in FIG. 9A demonstrates that the reference antagonist antibody inhibits the BTLA/HVEM complex FRET signal in a concentration-dependent manner with an IC₅₀ of approximately 0.65 μg/ml.

In FIG. 9B a 293c18 cell clone stably expressing full length human BTLA and human HVEM was harvested with Accutase solution (Millipore Sigma/Sigma-Aldrich), washed in FACS buffer, and plated in white U-bottom 96-well plates (2×10⁵ cells/well). Cells were centrifuged, FRET acceptor antibody APC-anti-HVEM (clone 122, BioLegend) or APC-mouse IgG1 isotype control antibody serially diluted 3-fold at the indicated concentrations was added to the plate. The biotinylated FRET donor antibodies were pre-complexed at 10 μg/ml with 0.025 μg/ml Streptavidin-Eu (LANCE Eu-W8044-Streptavidin AD0060, PerkinElmer) by incubating for 20 minutes at room temperature. Biotin conjugated FRET donor antibodies tested were biotin-6G3 IgG4 (APE13124.01, which was biotin conjugated APE12839.05 (H Chain SEQ ID NO: 144, L Chain SEQ ID NO: 174)), a biotin-reference antagonist IgG4 antibody, and a biotin-human IgG4 isotype control antibody specific for hen egg lysozyme. The donor biotin-antibody/Streptavidin-Eu complex final concentration in all wells was 0.3 μg/ml antibody and 0.75 ng/ml Streptavidin-Eu. Plates were incubated 24 hours at 4° C., washed in cold FACS buffer and fluorescence of samples was read on an En Vision Multimode Plater Reader (PerkinElmer). The ratio of fluorescence at 665 nm/615 nm was graphed in GraphPad Prism (GraphPad Software). Each point is the mean±SEM of 2 replicate wells.

The FRET assay shown in FIG. 9B demonstrates that in the presence of the biotin-6G3 antibody disclosed herein complexed with Streptavidin-Eu, increasing concentrations of APC-anti-HVEM generate a concentration-dependent increasing FRET signal. This example demonstrates that when the 6G3 antibody binds to cell surface BTLA, BTLA is still capable of forming a complex with HVEM on the same cell surface.

The FRET assay shown in FIG. 9B demonstrates that in the presence of the biotin-reference antagonist antibody complexed with Streptavidin-EU the BTLA/HVEM complex FRET signal is not detectable and is similar to that of the biotin-isotype control antibody. This example demonstrates that the reference BTLA antagonist antibody disrupts the BTLA-HVEM complex on the same cell surface.

### Example 11

This example demonstrates that, when BTLA and HVEM are expressed on different cells, the 6G3 antibody disclosed herein partially inhibits BTLA-induced HVEM signaling in an NF-κB luciferase reporter assay.

293c18 cells stably expressing full-length human HVEM and an NF-κB-luciferase reporter construct were generated and designated HVEM/NF-κB luciferase, clone 11. 293c18 cells stably expressing full-length human BTLA were generated, single cell cloned, and designated huBTLA 293c18, clone 2. Human BTLA 293c18 cells were harvested with Accutase solution (Sigma-Aldrich/Millipore Sigma), resuspended in DMEM supplemented with 10% FBS, and plated (0.5×10⁴ cells/well) in a flat-bottom 96-well plate. The indicated concentrations of the 6G3 IgG4 (APE13308.03) antibody manufactured from a pool of stably transfected CHO-K1 cells, human IgG4 isotype control antibody specific for hen egg lysozyme, or IgG4 reference anti-BTLA antagonist antibody) were added to the cells. After a 30-minute incubation at room temperature, HVEM/NF-κB cells were harvested and added (5×10⁴ cells/well). After 5 hours at 37° C., 5% CO₂, an equal volume of Steady-Glo Luciferase Assay System (Promega) was added to the wells and allowed to incubate at room temperature for 10 minutes. Luminescence of samples was read on a GloMax Navigator Microplate Luminometer (Promega) with a measurement time of 0.3 seconds. Luminescence in relative light units (RLU) was graphed and curves fit with a log (agonist) vs. response (three parameters) least squares fit in GraphPad Prism (GraphPad Software).

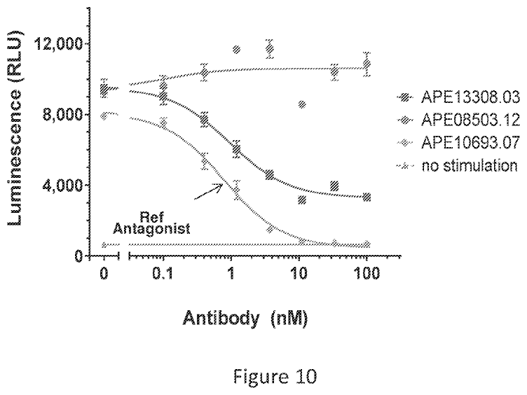

The NF-κB luciferase reporter assay shown in FIG. 10 demonstrates that the 6G3 antibody disclosed herein partially inhibits HVEM-dependent NF-κB signaling in a concentration-dependent manner in response to BTLA 293c18 cells, when HVEM and BTLA are expressed on different cells. This suggests that, under these conditions, the 6G3 antibody disclosed herein may partially inhibit HVEM signaling when BTLA is on a different cell. The same result is obtained when the human BTLA 293c18 cells are fixed with paraformaldehyde prior to incubation with the 6G3 antibody disclosed herein or when cells are treated with an inhibitor of dynamin GTPase that blocks endocytosis.

The NF-κB luciferase reporter assay shown in FIG. 10 demonstrates that addition of a reference antagonist anti-BTLA antibody results in a concentration-dependent complete inhibition of BTLA-mediated HVEM-dependent NF-κB signaling when HVEM and BTLA are expressed on different cells.

### Example 12

This example demonstrates the direct BTLA agonist activity of the 6G3 antibody disclosed herein in an SHP2 recruitment PathHunter Jurkat BTLA signaling assay.

A clonal Jurkat cell line stably expressing β-galactosidase enzyme donor (ED)-tagged human BTLA and β-galactosidase enzyme acceptor (EA)-tagged human SHP2 was generated at Eurofins DiscoverX (Fremont, CA) and designated Jurkat BTLA-ED SHP2-EA cells. For the assay setup Jurkat BTLA-ED SHP2-EA cells were harvested and plated in a 96-well plate (2×10⁴ cells/well). Antibody dilutions were prepared in a separate plate. Antibodies and proteins tested were the 6G3 IgG4 (APE13308.03) antibody manufactured from a pool of stably transfected CHO-K1 cells, human IgG4 isotype control antibody specific for hen egg lysozyme, a reference BTLA antagonist IgG4 antibody, and a soluble complex of human HVEM-IgG1 Fc/trimeric LIGHT (APE11989.16 and APE07872.05 at a molar ratio of 1:1.1). Antibodies and proteins were added to the cell assay plate and incubated for 2 hours at room temperature. PathHunter Bioassay Detection reagent was added to all wells and incubated at room temperature for 20 minutes. Bioassay Detection Reagent 2 was then added to all the wells and incubated at room temperature for 1 hour prior to measuring the luminescence signal on an En Vision Multimode Plater Reader (PerkinElmer) with a 0.1 second integration time. Data were graphed in GraphPad Prism (GraphPad Software). EC₅₀ values were calculated using a sigmoidal dose response curve fit with variable slope (four parameter) with no constraints and using a least squares fit method. Data represent the mean (=standard deviation) of triplicate samples for each point. Assays and data analyses were performed at Eurofins DiscoverX (Fremont, CA) under Project ID: DRX-ANAB-190724.

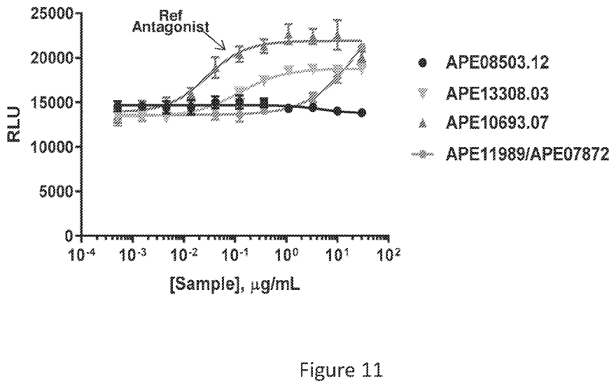

The SHP2 recruitment PathHunter Jurkat BTLA signaling assay shown in FIG. 11 demonstrates that the 6G3 antibody disclosed herein, as a soluble antibody, has direct BTLA agonist activity and induces concentration-dependent low magnitude BTLA signaling (EC₅₀=125 ng/ml). By inducing SHP2 recruitment to the BTLA cytoplasmic domain the 6G3 antibody disclosed herein may function by initiating inhibitory signaling in activated T and B cells.

The SHP2 recruitment PathHunter Jurkat BTLA signaling assay shown in FIG. 11 demonstrates that the reference BTLA antagonist antibody as a soluble antibody has direct BTLA agonist activity and induces concentration-dependent BTLA signaling (EC₅₀=28.8 ng/ml) with a higher maximum signal than the 6G3 antibody. In the SHP2 recruitment PathHunter Jurkat BTLA signaling assay shown in FIG. 11 the soluble HVEM/LIGHT complex induces weak concentration-dependent induced BTLA signaling that does not saturate likely due to the low affinity of soluble HVEM for BTLA as compared with the antibodies.

### Example 13

This example demonstrates in an SHP2 recruitment PathHunter Jurkat BTLA signaling assay that the 6G3 antibody disclosed herein does not inhibit BTLA signaling induced by HVEM on a transfected U-2 OS cell line.

A clonal Jurkat cell line stably expressing β-galactosidase enzyme donor (ED)-tagged human BTLA and β-galactosidase enzyme acceptor (EA)-tagged human SHP2 (Jurkat BTLA-ED SHP2-EA cells) and a U-2 OS osteosarcoma cell line stably expressing human HVEM (U-2 OS hHVEM cells) were generated at Eurofins DiscoverX (Fremont, CA). For the assay setup, Jurkat BTLA-ED SHP2-EA cells were harvested and plated in a 96-well plate (2×10⁴ cells/well). Antibody dilutions were prepared in a separate plate. Antibodies tested were the 6G3 IgG4 (APE13308.03) antibody manufactured from a pool of stably transfected CHO-K1 cells, human IgG4 isotype control antibody specific for hen egg lysozyme, and a reference BTLA antagonist IgG4 antibody. Antibodies at the indicated concentrations were added to the assay plate and incubated for 1 hour in a humidified incubator at 37° C., 5% CO₂. U-2 OS hHVEM cells were harvested, resuspended, and added to the assay plate (5×10⁴ cells/well) with the Jurkat BTLA-ED SHP2-EA cells and incubated for 2 hours at room temperature. PathHunter Bioassay Detection reagent was added to all wells and incubated at room temperature for 30 minutes. Bioassay Detection Reagent 2 was then added to all the wells and incubated at room temperature for 1 hour prior to measuring the luminescence signal on an En Vision Multimode Plater Reader (PerkinElmer) with a 0.1 second integration time. Data were graphed in GraphPad Prism (GraphPad Software); IC₅₀ values were calculated using a sigmoidal dose response curve fit with variable slope (four parameter) with no constraints and using a least squares fit method. Data represent the mean (±standard deviation) of triplicate samples for each point. Assays and data analyses were performed at Eurofins DiscoverX (Fremont, CA) under Project ID: DRX-ANAB-191210.

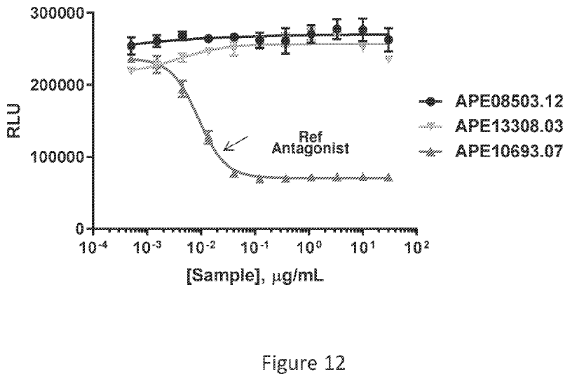

The SHP2 recruitment PathHunter Jurkat BTLA signaling assay shown in FIG. 12 demonstrates that the 6G3 antibody disclosed herein has no effect on SHP2 recruitment to BTLA induced by HVEM on a different cell.

The SHP2 recruitment PathHunter Jurkat BTLA signaling assay shown in FIG. 12 demonstrates that the reference BTLA antagonist antibody exhibits potent concentration-dependent inhibition of BTLA signaling induced by HVEM on a different cell (IC₅₀=8.9 ng/ml).

### Example 14

This example demonstrates the increased direct BTLA agonist activity of the 6G3 antibody disclosed herein in an SHP2 recruitment PathHunter Jurkat BTLA signaling assay with addition of FcγRIa (CD64a) transfected U-2 OS cells to provide FcγR engagement.

A clonal Jurkat cell line stably expressing β-galactosidase enzyme donor (ED)-tagged human BTLA and β-galactosidase enzyme acceptor (EA)-tagged human SHP2 (Jurkat BTLA-ED SHP2-EA cells) and a U-2 OS osteosarcoma cell line stably expressing human FcγRIa/CD64a (U-2 OS hFcγRIa cells) were generated at Eurofins DiscoverX (Fremont, CA). U-2 OS hFcγRIa cells were harvested and plated in a 96-well plate (1×10⁴ cells/well). Antibody dilutions were prepared in a separate plate, added to the assay plate, and incubated for 1 hour in a humidified incubator at 37° C., 5% CO₂. Antibodies tested were the 6G3 IgG4 (APE13308.03) antibody manufactured from a pool of stably transfected CHO-K1 cells, human IgG4 isotype control antibody specific for hen egg lysozyme, and a reference BTLA antagonist IgG4 antibody. Jurkat BTLA-ED SHP2-EA cells were harvested and added to the assay plate (2×10⁴ cells/well) with the U-2 OS hFcγRIa cells. The plate was incubated for 2 hours at room temperature. PathHunter Bioassay Detection reagent was added to all wells and incubated at room temperature for 30 minutes. Bioassay Detection Reagent 2 was added to all the wells and incubated at room temperature for 1 hour prior to measuring the luminescence signal on an En Vision Multimode Plater Reader (PerkinElmer) with a 0.1 second integration time. Data were graphed in GraphPad Prism (GraphPad Software); EC₅₀ values were calculated using a sigmoidal dose response curve fit with variable slope (four parameter) with no constraints and using a least squares fit method. Data represent the mean (=standard deviation) of triplicate samples for each point. Assays and data analyses were performed at Eurofins DiscoverX (Fremont, CA) under Project ID: DRX-ANAB-191210.

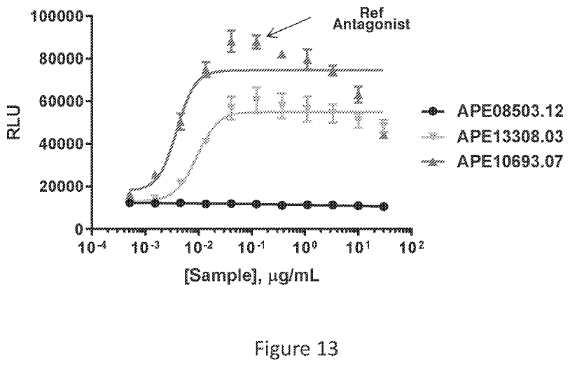

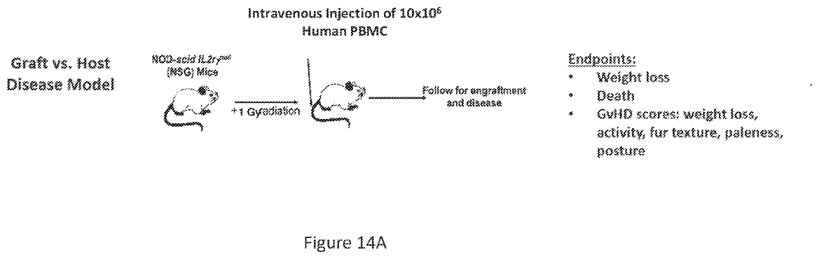

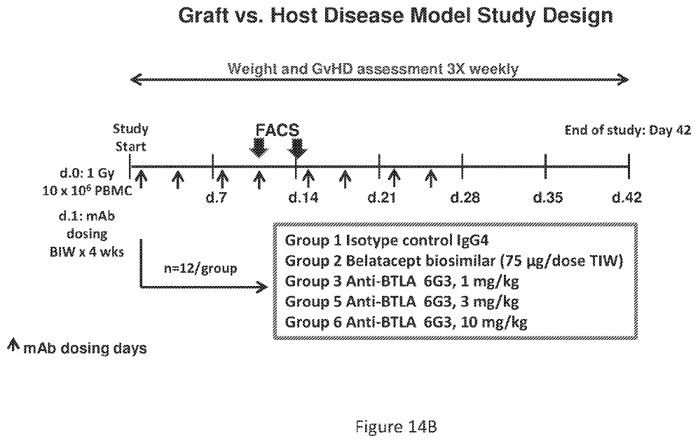

The SHP2 recruitment PathHunter Jurkat BTLA signaling assay shown in FIG. 13 demonstrates that the 6G3 antibody disclosed herein, as a soluble antibody in the presence of cells providing FcγRIa engagement, has increased direct BTLA agonist activity and induces concentration-dependent BTLA signaling (EC₅₀=9.3 ng/ml).

The SHP2 recruitment PathHunter Jurkat BTLA signaling assay shown in FIG. 13 demonstrates that the reference BTLA antagonist antibody as a soluble antibody in the presence of cells providing FcγRIa engagement has direct BTLA agonist activity and induces concentration-dependent BTLA signaling (EC₅₀=3.9 ng/ml).

The SHP2 recruitment PathHunter Jurkat BTLA signaling assay shown in FIG. 13 demonstrates that both the 6G3 antibody disclosed herein and the reference BTLA antagonist antibody show increased potency agonist activity in the presence of cell-associated FcγRIa as compared to the Jurkat BTLA signaling assay without FcγRIa (6G3˜13-fold more potent; reference BTLA antagonist ˜7.4-fold more potent) (compare FIG. 11 and FIG. 13). While soluble 6G3 antibody disclosed herein may induce SHP2 recruitment to the BTLA cytoplasmic domain and initiate inhibitory signaling in activated T and B cells, the potential for FcγRIa-bound 6G3 antibody to induce direct inhibitory signaling may be enhanced.

### Example 15

This example demonstrates that the 6G3 antibody disclosed herein shows efficacy in vivo in a xenogeneic NSG/Hu-PBMC Graft vs. Host Disease (GvHD) model when dosed twice weekly at 1 mg/kg, 3 mg/kg, or 10 mg/kg for 4 weeks.

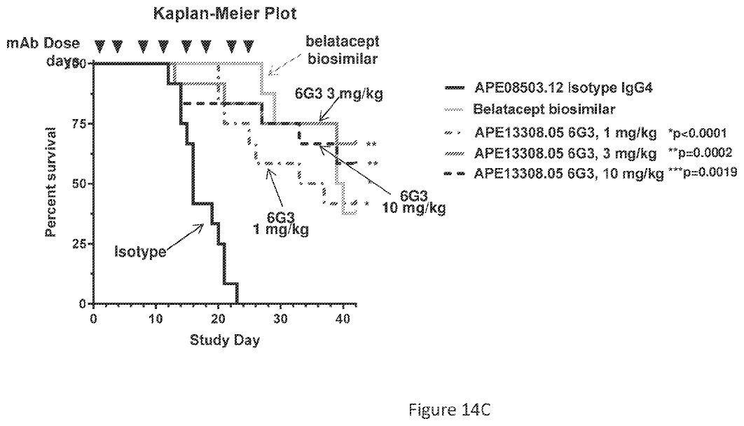

A xenogeneic NSG/Hu-PBMC GvHD model testing the efficacy of the 6G3 anti-BTLA antibody disclosed herein was performed at The Jackson Laboratory JAX® In Vivo Pharmacology Services (Sacramento, CA). NOD-scid IL2rγ^{null} (NSG) mice were irradiated with 1 Gy followed by intravenous injection of 10×10⁶ human PBMCs in each mouse as illustrated in FIG. 14A. Antibodies (human IgG4 isotype control antibody specific for hen egg lysozyme; and 6G3 IgG4 APE13308.05 antibody manufactured from a pool of stably transfected CHO-K1 cells) were dosed intraperitoneally twice weekly for 4 weeks starting the day following PBMC injection. The 6G3 antibody disclosed herein was dosed at either 1 mg/kg, 3 mg/kg, or 10 mg/kg, and the human IgG4 isotype control antibody was dosed at 10 mg/kg. There were 12 animals/group in each antibody treatment group. Belatacept biosimilar positive control was dosed intraperitoneally at 75 μg/mouse three times weekly for 4 weeks. There were 8 animals in the belatacept biosimilar treatment group. Dosing regimens and dose groups in the study are shown in FIG. 14B. Over the course of the 42-day study, disease was monitored three times weekly by body weight loss, death, and GvHD scores measuring: weight loss, activity, fur texture, paleness, and posture. Animals exhibiting more than 10% body weight loss were disease monitored daily, and animals exhibiting more than 20% body weight loss from starting weight were euthanized. Survival included animals found dead and animals removed from the study due to endpoints defined in the study protocol at The Jackson Laboratory JAX® In Vivo Pharmacology Services. Survival data were graphed in GraphPad Prism (GraphPad Software) using a Kaplan-Meier survival analysis, which calculated median survival of each group. Statistical significance of the treatment groups in pairwise comparisons with the isotype control group were in GraphPad Prism using a Gehan-Breslow-Wilcoxon test for calculation of p-values.

The survival results for the GvHD study shown in FIG. 14C demonstrate that the 6G3 anti-BTLA antibody disclosed herein shows highly statistically significant efficacy in prolonging survival at all doses tested as compared with the isotype control antibody. Efficacy of the 6G3 antibody was dose responsive, with the 1 mg/kg dose treatment group exhibiting decreased survival as compared with the 3 mg/kg and 10 mg/kg treatment groups. Median survival times over the course of the study were 16 days for the isotype control group, 35 days for the 1 mg/kg 6G3 treatment group, and not defined for the 3 mg/kg and 10 mg/kg 6G3 treatment groups. A reference antagonist anti-BTLA antibody was dosed at 10 mg/kg as a secondary control. No survival benefit was observed in animals treated with the reference antagonist antibody compared to isotype control treated animals.

### Example 16

This example illustrates the qualification of a 96 well, electrochemiluminescence (ECL) sandwich assay for the detection of BTLA after dosing of a 6G3 IgG4 anti-BTLA antibody in cynomolgus monkey serum.

During method development, assay parameters such as capture reagent concentration, MRD, assay buffer type and detect reagent concentrations were established. Method qualification of the assay was then performed by evaluating intra-assay and inter-assay precision and accuracy, dilution linearity, specificity and freeze thaw stability. The final method will be used to analyze samples from non-GLP single or multi-dose PK, TK and tolerability studies in cynomolgus monkeys.

96-well MSD Standard Bind assay plates (MSD Part #L15XA-3) were coated overnight at 4° C. with 50 μL of 1.0 μg/mL Anti-BTLA clone 10D8 (APE10134). The following day the coated plate was washed 3 times with 1×PBST and blocked with 250 μL Blocking Buffer for 60 to 120 minutes. The calibration curve range for BTLA was 500-7.8 ng/ml with a quantitative range 500-7.8 ng/mL. Standards were prepared using a 2-fold dilution series in 100% cynomolgus serum. Quality controls (QCs) were also prepared and frozen in 100% cynomolgus serum at five different concentrations spanning the quantitative range. All standards, samples and controls were then diluted in assay buffer containing 100 μg/ml 6G3 IgG4 at the minimum required dilution (MRD) of 1:10 and incubated for 1 hour at room temperature on a shaker. After blocking, plates were washed with 1×PBST and 50 μL of the diluted standards, samples and QCs were incubated for 2 hours at room temperature on a shaker between 400-500 rpms. After sample incubation, plates were washed 3 times in 1×PBST and 50 μL of 0.25 μg/mL Biotinylated anti-BTLA polyclonal detection antibody (PA5-95592 by ThermoFisher) was added to each well and incubated for 1 hour at room temperature on a shaker. Plates were then washed 3 times with 1×PBST. Next, 50 μL per well of 0.2 μg/mL Strepavidin-SulfoTag secondary detection reagent was added to each well and incubated for 30 minutes at room temperature on a shaker. Plates were then washed a final 3 times with 1×PBST. 150 μL per well of 2×MSD Read Buffer T was added to each well and the plate was read on MSD Quickplex Plate Reader. Standards, samples and controls replicates were tested in duplicate and serum concentrations back calculated based on the reference standard curves for the lead candidate using SoftMax Pro 7.01 and a 4 parameter curve fit with 1/y\hat{ }2 weighting.

Before beginning method qualification, serum matrix interference was evaluated for the 6G3 IgG4 antibody. Matrix effects were not observed and a minimum required dilution (MRD) of 1:10 was selected.

Antibody 10D8 (APE10134) was used as a capture antibody. It was discovered that the binding of 10D8 to BTLA was increased in the presence of the 6G3 IgG4 antibody potentially through conformational changes of BTLA when bound by 6G3 IgG4 in Cyno serum. In order to normalize the putative conformational changes of BTLA, 100 μg/ml 6G3 was added to the dilution buffer and all standards, samples and controls and diluted according to MRD of 1:10 and incubated for 1 hour at room temperature on a shaker prior to adding samples to 10D8 coated MSD plate.

Standard curves with 500, 50, 5, 0.5, & 0 μg/ml 6G3 IgG4 were diluted in assay buffer containing 100 μg/ml 6G3 IgG4 at MRD 1:10 to assess (% RE) and precision (% CV) of BTLA. All five standard curve conditions incubated with 100 μg/ml 6G3 IgG4 had standard concentrations in the quantitative assay range and passed the acceptance criteria of the mean recovered (% RE) concentration being within 20% of the nominal concentration. The averaged results of the precision (% CV) passed acceptance criteria and did not exceed 20% for all samples. The percent total error (% TE) did not exceed 30% for BTLA.

Inter-assay accuracy (% RE) and precision (% CV) was assessed over six assay runs performed on 2 different days and included a calibration curve on each plate and the five levels of QCs that defined the quantitative range. The five levels of QCs assessed were ULOQ (500 ng/mL), HQC (400 ng/ml), MQC (62.4 ng/mL), LQC (15.6 ng/mL) and LLOQ (7.8 ng/ml). At each QC level per run a total of 3 independent replicates were analyzed in duplicate. The average results of the accuracy (% RE) for all controls and standard concentrations in the quantitative assay range passed the acceptance criteria of the mean recovered concentration being within 20% of the nominal concentration. The averaged results of the precision (% CV) passed acceptance criteria and did not exceed 20% for all samples. The percent total error (% TE) did not exceed 30% for BTLA for either standard curves or for quality controls for any concentrations in the quantitative range of the assay.

The intra-assay accuracy (% RE) and precision (% CV) of the method was assessed in a single run with six independent replicates of the five levels of QCs. The average results of the accuracy (% RE) for the controls passed the acceptance criteria of the mean recovered concentration being within 20% of the nominal concentration. The averaged results of the precision (% CV) passed acceptance criteria and did not exceed 20%.

Dilution Linearity was assessed by performing a range of dilutions on samples spiked with concentrations of BTLA that were above the ULOQ to demonstrate that high concentrations of test article could be diluted into the quantitative range and that the assay did not have a prozone effect. The samples where diluted into the quantitative range of the assay and back calculated concentrations were assessed. The averaged results indicate that it is possible to dilute samples down into the quantitative range with a dilution factor up to 1:1600. There was no prozone “hook effect” present at the concentrations evaluated.

The results show the assay to be sensitive and reproducible to evaluate BTLA concentrations in serum collected from cynomolgus monkey studies dosed with 6G3 IgG4.

### Example 17

The following example illustrates the use of an Electrochemiluminescence (ECL) sandwich assay to quantitatively determine soluble BTLA in cynomolgus monkey serum and NOD scid mouse plasma.

96-well MSD Standard Bind assay plates (MSD Part #L15XA-3) were coated overnight at 4° C. with 50 μL of 1.0 μg/mL anti-BTLA capture reagent clone 10D8 (APE10134). The following day the coated plate was washed 3 times with 1×PBST and blocked with 250 μL Blocking Buffer for 60 to 120 minutes. The calibration curve range for sBTLA was 7.8-500 ng/mL with a quantitative range of 7.8-500 ng/ml in cynomolgus monkey serum, and 2.0-1000 ng/mL for both calibration and quantitative range in CD1 mouse plasma. Standards were prepared using a 2-fold dilution series in 100% species-specific matrix. Quality controls (QCs) were also prepared and frozen in 100% species-specific matrix at five different concentrations spanning the quantitative range. All standards, samples and controls were then diluted in assay buffer containing 100 μg/mL 6G3 at the minimum required dilution (MRD) of 1:10 and incubated for 1 hour at room temperature on a shaker.

After blocking, plates were washed with 1×PBST, and 50 μL of the diluted standards, samples and QCs were incubated for 2 hours at room temperature on a shaker between 400-500 rpms. After sample incubation, plates were washed 3 times in 1×PBST and 50 μL of 0.25 μg/mL Biotinylated anti-BTLA clone PA5-95592 (VC2963104B) detection antibody was added to each well and incubated for 1 hour at room temperature on a shaker. Plates were then washed 3 times with 1×PBST. Next, 50 μL per well of 0.2 μg/mL Strepavidin-SulfoTag secondary detection reagent was added to each well and incubated for 30 minutes at room temperature on a shaker. Plates were then washed a final 3 times with 1×PBST. 150 μL per well of 2×MSD Read Buffer T was added to each well and the plate was read on MSD Quickplex Plate Reader. Standards, samples and controls replicates were tested in duplicate and concentrations back calculated based on the reference standard curves for BTLA using SoftMax Pro 7.01 and a 4-parameter curve fit with 1/y\hat{ }2 weighting.

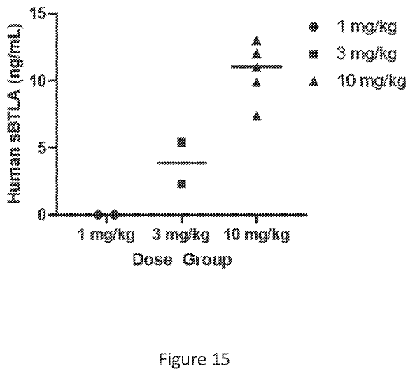

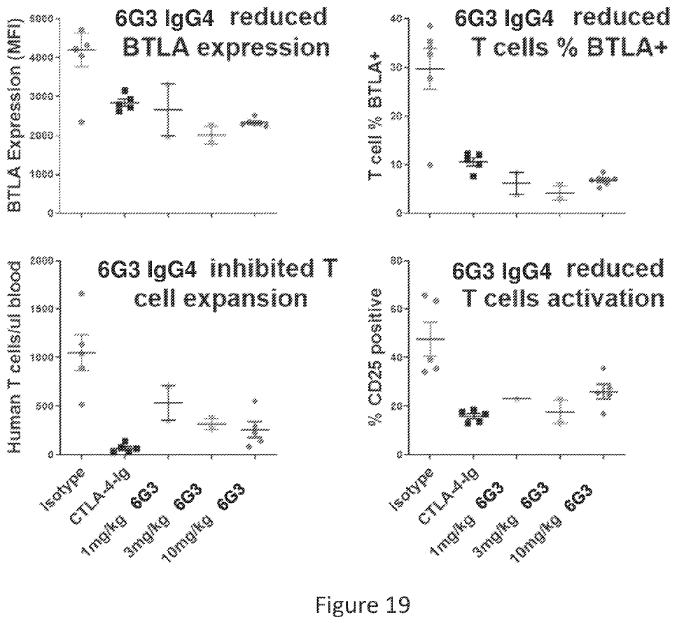

The human PBMC engrafted NOD Scid mice (n=19) were dosed with the 6G3 IgG4 antibody (APE13308) in three dose groups (1, 3, and 10 mg/kg IP). Animals dosed with isotype control IgG4 (10 mg/kg) or CTLA-4-Ig (75 μg) served as controls. Animals were dosed twice per week, and plasma samples were collected mid-point in the study via cardiac bleed. The results are presented in FIG. 15. In addition, circulating human T cells were analyzed by flow cytometry to characterize BTLA expression, enumerate human T cells, and characterize the activation marker CD25. The results are shown in FIG. 19. 6G3 IgG4 reduced BTLA expression on human T cells, inhibited T cell expansion in a dose-dependent manner and reduced expression of the activation marker CD25 at all doses.

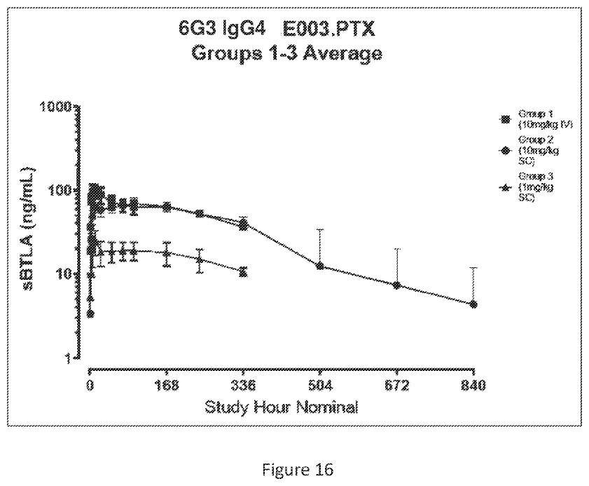

Blood samples were analyzed from Cynomolgus monkeys from two studies. The first study (n=180) consisted of three dosing groups (10 mg/kg IV; 10 mg/kg SC; and 1 mg/kg SC). All animals were administered a single dose of 6G3 IgG4 either IV or SC and blood samples were collected from all animals in all groups predose, 3, 6, 12, 24, 48, 72, 96, 168, 240, 336, 504, 672, and 840 hours postdose. Pre-dose samples and vehicle control dosed animals served as controls. The results are presented in FIG. 16.

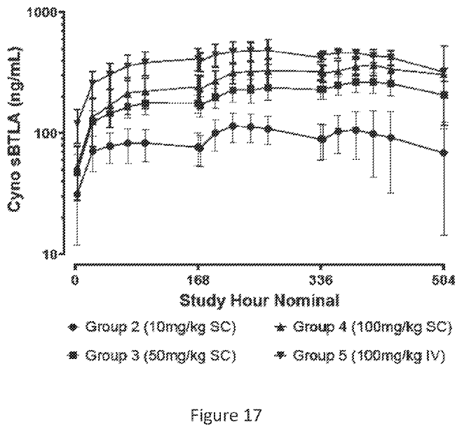

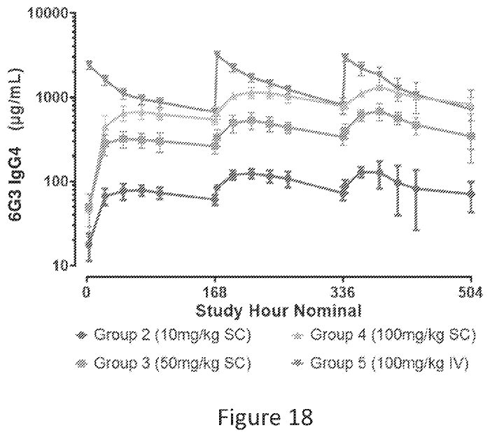

In the second study, cynomolgus monkeys (n=380) were dosed with 6G3 in four dose groups (10, 50, and 100 mg/kg SC, and 100 mg/kg IV). All animals were administered a weekly dose of 6G3 IgG4 on Days 1, 8, and 15 either IV or SC and blood samples were collected from all animals in all groups on Days 1, 8, and 15: Predose, 3, 24, 48, 72, 96 hours postdose. The results are presented in FIG. 17. Serum levels of 6G3 IgG4 were also measured. The results are presented in FIG. 18.

No measurable levels of sBTLA were detected in samples from human PBMC engrafted NOD scid mice dosed with IgG4 Isotype control or CTLA-4-Ig, or in cynomolgus monkey pre-dose or vehicle control treated samples from the cynomolgus monkey studies. Shed sBTLA was detected in serum samples from all cynomolgus monkeys and all plasma samples from human PBMC engrafted NOD scid mice dosed with 6G3 IgG4, with the exception of the lowest dose group (1 mg/kg) in the mouse study. The results suggest that BTLA is shed from the surface of B and T cells and that sBTLA is a pharmacodynamic marker of 6G3 IgG4 activity in vivo.

### Example 18

This Example demonstrates that 6G3 IgG4 achieves receptor occupancy and reduces BTLA expression on T and B cells in cynomolgus monkeys.

Flow cytometry was used to analyze cynomolgus monkey peripheral blood for the effects of multiple doses of 6G3 IgG4 in a dose range finding study (DRFS). Animals were dosed with 6G3 IgG4 in four different treatment groups (10 mg/kg SC, 50 mg/kg SC, 100 mg/kg SC, 100 mg/kg IV) and one control group.

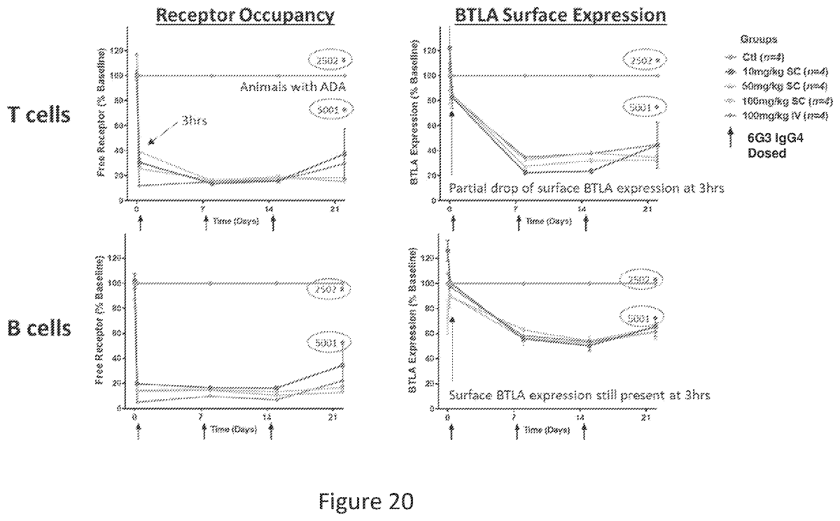

6G3 IgG4 caused no significant changes in T cell, B cell and NK cell absolute counts, or percent distribution compared to vehicle control tested animals. Binding of fluorochrome labeled drug (6G3 IgG4-DyL488) was abrogated in all animals dosed with 6G3 IgG4 compared to the vehicle control treated group, demonstrating receptor occupancy of BLTA with 6G3 IgG4 to be >80% for T cells and ˜70% for B cells, as shown in FIG. 20.

All animals doses with 6G3 IgG4 had reduced BTLA surface expression on T and B cells when detected with a fluorochrome labeled non-competing anti-BTLA antibody (clone 10D8) compared to vehicle control treated animals. Surface expression of BTLA on T and B cells was reduced ˜75% and ˜50%, respectively, as shown in FIG. 20.

It was found that there was a difference in the kinetics of receptor occupancy on T and B cells, which occurred very rapidly, compared to the loss of BTLA expression on T and B cells, which occurred relatively slowly. This suggests an 6G3 IgG4 dependent mechanism leads to BTLA shedding. Two animals that developed anti-drug antibodies (ADA) demonstrated some loss of receptor occupancy and recovery of BTLA expression on T and B cells.

### Example 19

This Example demonstrates that the BTLA extracellular domain (ECD) is cleaved by the serine protease 3 at a proposed recognition site near the transmembrane domain.

Proteinase 3 (PR3) is a neutrophil serine protease that is released into the extracellular space upon neutrophil activation. PR3 has previously been shown to cleave the checkpoint receptor T-cell immunoglobulin and mucin domain 3 (TIM-3), reducing the levels of TIM-3 on the surface of cells.

Six different lanes were incubated for up to 60 minutes with purified recombinant PR3 and/or BTLA-ECD. They consisted of (1) PR3 control, (2) PR3 60 m control, (3) BTLA control, (4) PR3+BTLA Om, (5) PR3+BTLA 30 m, and (5) PR3+BTLA 60 m.

Following co-incubation, soluble BLTA-ECD was cleaved into multiple smaller fragments demonstrating that PR3 has the ability to cleave BTLA. An analysis of the sequence proposed at least one PR2 recognition motif towards the C-terminus of the BTLA-ECD.

### Example 20

This Example demonstrates that 6G3 IgG4 reduces T-cell proliferation and surface BTLA expression in healthy controls (HC) and atopic dermatitis (AD) donors.

HC or AD donors' PBMCs were labeled with 0.5 μM CFSE, and then were stimulated with soluble anti-CD3 (0.5 ng/ml, Biolegend, cat #300332) and soluble anti-CD28 (0.5 ng/ml, Biolegend, cat #302943) in the presence or absence of 100 nM of 6G3 IgG4 or isotype control (IgG4-HyHel) for 72 hours. Proliferating cells were determined by CFSE dilution.

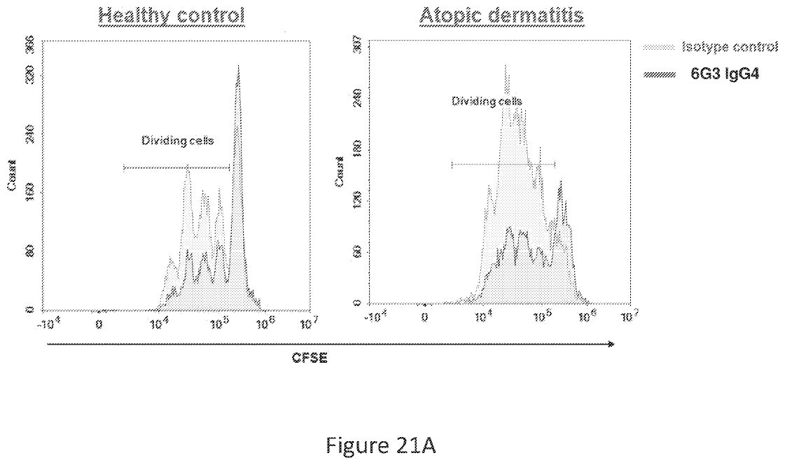

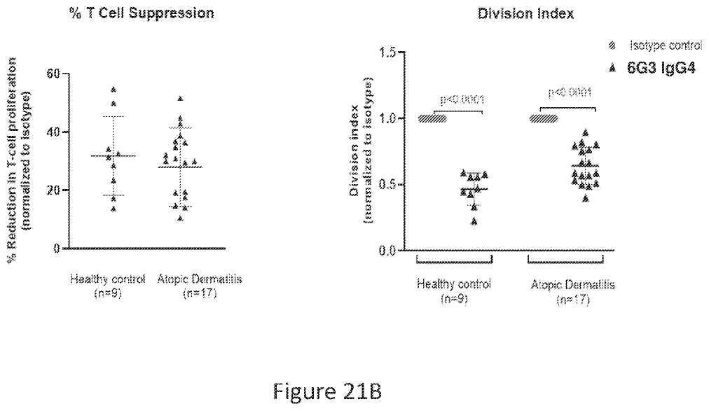

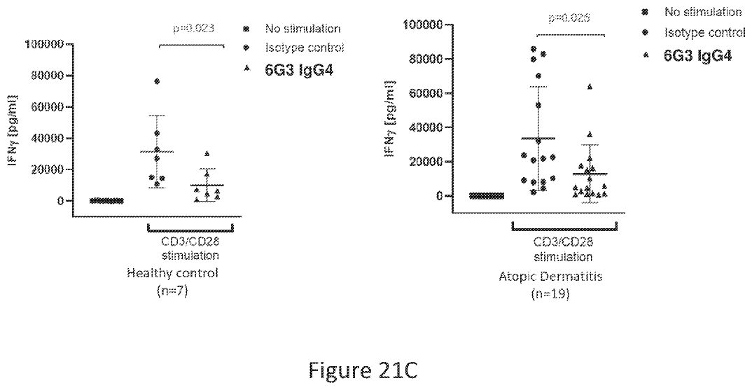

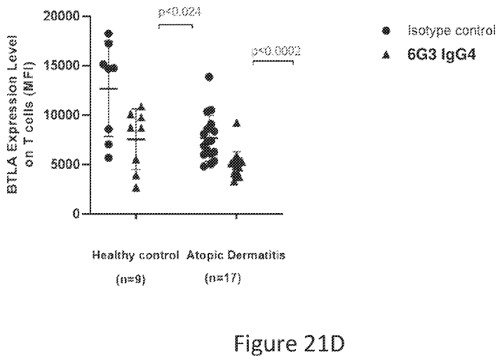

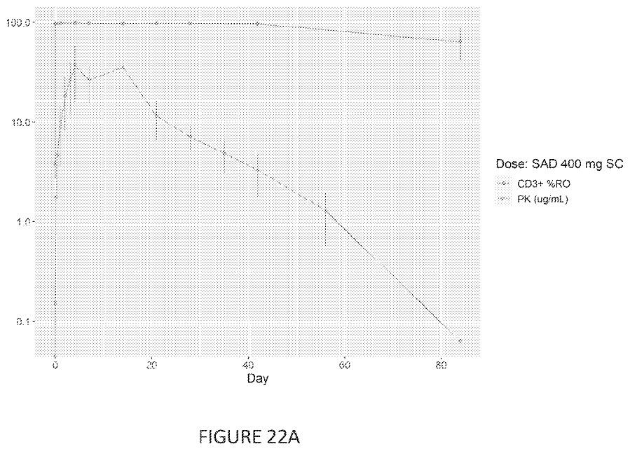

FIG. 21A provides CFSE histograms of HC and AD donors shown as the overlaid histograms of isotype control onto 6G3 IgG4 treated CD3+ T-cells. FIG. 21B shows T cell proliferation percentage reduction in proliferation (left) and division index (right). Division index is the sum of the number of divisions in each generation divided by the number of original cells, and is calculated by NovoExpress software automated cell cycle proliferation analysis. IFNγ levels of HC and AD donors PBMC culture supernatant were measured by Mesoscale MDS assay after 72 hour anti-CD3 and anti-CD28 stimulation in the presence or absence of 100 nM of 6G3 IgG4 or isotype control. Results are shown in FIG. 21C. FIG. 21D shows BTLA surface expression (plotted as mean fluorescence intensity (MFI)) on HC and AD donors CD3+ T-cells determined by AF647 conjugated anti-BTLA (clone #10D8, AnaptysBio.)

The results show that 6G3 IgG4 reduces T-cell proliferation and surface BTLA expression as compared to controls.

### Example 21

This Example demonstrates that 6G3 IgG4 achieves high BTLA receptor occupancy at safe dosages in human patients.

96 subjects were randomized/treated in the study, including 72 subjects in single ascending dose (SAD) cohorts and 24 in multiple ascending dose (MAD) cohorts. SAD cohorts received a single dose of 7 mg, 20 mg, 70 mg, 200 mg, 400 mg, or 800 mg, either via intravenous (IV) or subcutaneous (SC) administration. MAD cohorts received weekly SC administration of 70 mg, 200 mg, or 400 mg over 4 weeks. Each cohort contained 8 subjects, 6 dosed with 6G3 IgG4 antibody, and 2 with placebo. 71 of the 72 SAD subjects completed the study, and 21/24 MAD subjects completed the study. The treatment was well tolerated, with no safety signals evident.

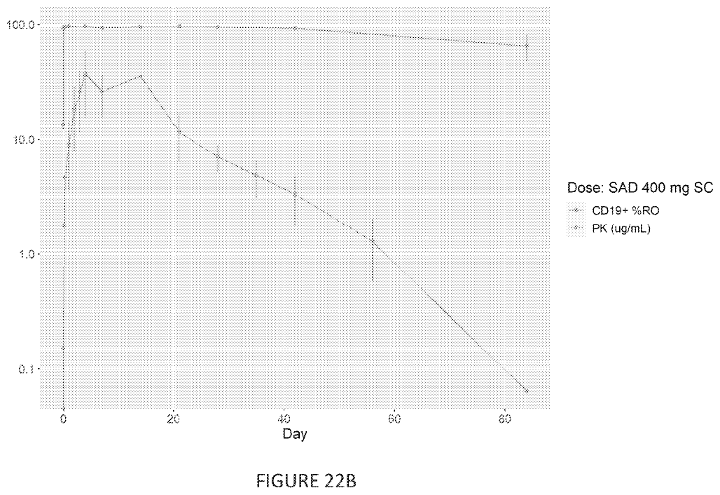

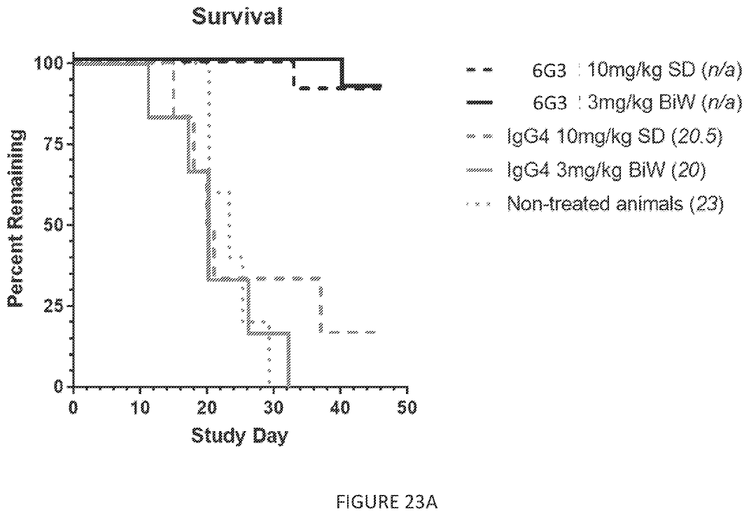

BTLA receptor occupancy on CD3+ T-cells and CD19+ B-cells was determined. Some results are presented in FIGS. 22A and B. It was found that receptor occupancy maximized at 7 mg IV or 20 mg SC, with a dose-dependent increase in duration. Single dosing of 200 mg IV and 400 mg SC provided 90% or greater receptor occupancy for more than 30 days. Maximal receptor occupancy can be sustained with multiple-dose SC administration as demonstrated by the MAD cohort.

### Example 22

This Example demonstrates that the 6G3 antibody disclosed herein shows efficacy in vivo in a Graft vs. Host Disease (GvHD) model when dosed with a single 10 mg/kg dose or biweekly 3 mg/kg dosing for 4 weeks.

Mice were engrafted with 1×10⁷ human peripheral blood mononuclear cells (PBMCs). They were then injected with either a single dose (SD), or biweekly (BiW) dosing for 4 weeks with 6G3 antibody or an IgG4 isotype control at 10 mg/kg, or 3 mg/kg. A control group of non-treated animals was also included to monitor engraftment levels and disease progression, excluding any treatment or placebo injection.

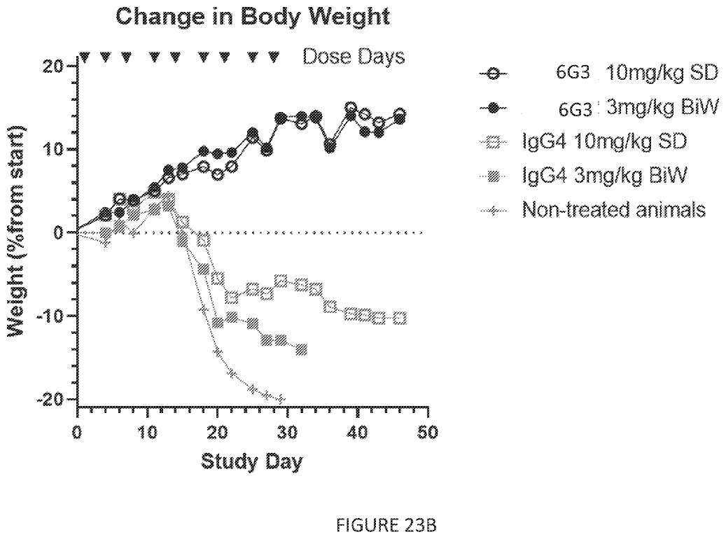

Animals treated with 6G3, either with a single 10 mg/kg dose or BiW 3 mg/kg dosing for 4 weeks showed clear survival benefit out to 46 days. Only one animal in each treatment group (SD, or BiW) succumbed to disease, with all other animals surviving to study day 46 (n=11). Animals treated with IgG4 isotype control with a single 10 mg/kg dose had a mean survival of 20.5 days, or 20 days with BiW 3 mg/kg dosing. Non-treated control animals had a median survival of 23 days. Results are shown in FIGS. 23A and 23B.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1.-8. (canceled)

9. A method of modulating BTLA signaling in a human subject, which method comprises administering a BTLA-binding agent to the mammal, wherein:
(i) the BTLA binding agent is administered in a weekly IV dose of at least 7 mg;
(ii) the BTLA binding agent is administered in a weekly SC dose of at least 20 mg;
(iii) the BTLA binding agent is administered in a single IV dose of at least 150 mg not more than once every 30 days;
(ii) the BTLA-binding agent is administered in a single subcutaneous dose of at least 300 mg not more than once every 30 days;
and wherein the BTLA binding agent comprises:
an immunoglobulin heavy chain variable region comprising:
(a) a CDRH1 comprising X¹SX²MN (SEQ ID NO: 195), wherein X¹ is N or T, and
X² is W, F, H, G, P, R, K, D, S, L, V, N, or Y
(b) a CDRH2 comprising RIYPX¹GX²X³DTNYX⁴GKFK (SEQ ID NO: 196),
wherein:
X¹ is absent or A;
X² is D, Y, Q, G, L, F, H, S, P, R, or T;
X³ is G, Y, A, F, S, D, V, T, E, K, or R; and
X⁴ is N, V, Q, R, A, F, Y, S, G, P, or T;
and
(c) a CDRH3 comprising X¹SGTFX²X³GNYX⁴X⁵YFDV (SEQ ID NO: 197),
wherein:
X¹ is K or R;
X² is N or D;
X³ is D, S, F, Y, F, V, S, G, T, R, I, L, or E;
X⁴ is R or H; and
X⁵ is W, R, F, L, N, Y, P, I, V, A, S, G, R, or K;
or comprising the immunoglobulin heavy chain variable region of any one of SEQ ID NOs: 43-156, or at least the CDRs thereof; or an amino acid sequence with at least 90% sequence identity thereto;
and
an immunoglobulin light chain variable region comprising:
(a) a CDRL1 comprising RX¹SENIYX²X³LA (SEQ ID NO: 198), wherein
X¹ is A or V;
X² is S or N; and
X³ is H, N, or Y;
(b) a CDRL2 comprising X¹AX²NLAX³ (SEQ ID NO: 199), wherein
X¹ is A or N;
X² is T or K; and
X³ is N, L, Q, G, F, V, K, S, R, T, H, or P; and
(c) a CDRL3 comprising QX¹FX²GPPLT (SEQ ID NO: 200), wherein
X¹ is L or H; and
X² is W, F, Y, P, N, V, K, M, L, G, or S;
or comprising the immunoglobulin light chain variable region of any of SEQ ID NOs: 157-192, or at least the CDRs thereof; or an amino acid sequence with at least 90% sequence identity thereto;
or a BTLA binding agent as set forth in Table 3.

10. The method of claim 9, wherein the mammal has a disorder that is responsive to BTLA modulation, and the disorder is thereby treated.

11. The method of claim 10, wherein the disorder is an autoimmune or inflammatory disease.

12. The method of claim 10 or 11, wherein the disease is rheumatoid arthritis, graft vs host disease, psoriasis, or inflammatory bowel disease.

13. The method of claim 9, wherein the immunoglobulin heavy chain polypeptide comprises the sequence:
(SEQ ID NO: 193)
QVQLVQSGAEVKKPGSSVKVSCKASGYX₁FSX₂SX₃MNWVRQAPG
OGLEWMGRIYPX₄GX₅X₆DTNYX₇GKFKGRVTITADKX₈TX₉TAY
MELX₁₀SLRSEX₁₁TAVX₁₂YX₁₃CAX₁₄SGTFX₁₅X₁₆GNYX₁₇
X₁₈YFDVWGKGTTVTVSSA,
wherein
X¹ is A or V;
X² is N or T;
X³ is W, F, H, G, P, R, K, D, S, L, V, N, or Y;
X⁴ is absent or A;
X⁵ is D, Y, Q, G, L, F, H, S, P, R, or T;
X⁶ is G, Y, A, F, S, D, V, T, E, K, or R;
X⁷ is N, V, Q, R, A, F, Y, S, G, P, or T;
X⁸ is S or F;
X⁹ is S, T, or N;
X¹⁰ is S or R;
X¹¹ is D or V;
X¹² is absent or Y;
X¹³ is Y or F;
X¹⁴ is K or R;
X¹⁵ is N or D;
X¹⁶ is D, S, F, Y, F, V, S, G, T, R, I, L, or E;
X¹⁷ is R or H; and
X¹⁸ is W, R, F, L, N, Y, P, I, V, A, S, G, R, or K.

14. The method of claim 9, wherein the immunoglobulin heavy chain polypeptide comprises any one of SEQ ID NOs: 43-156, or at least the CDRs thereof; or an amino acid sequence with at least 90% sequence identity thereto.

15. The method of claim 9, wherein the immunoglobulin light chain polypeptide comprises the sequence:
(SEQ ID NO: 194)
X₁IQX₂TQSPSSLSASVGDRVTITCRX₃SENIYX₄X₅LAWYQQKX₆
GKAPKLLIYX₇AX₈NLAX₉GVPSRFSGSGSGTDX₁₀TLTISSLQ
PEDFATYYCQX₁₁FX₁₂GPPLTFGGGTKVEIKR,
wherein
X¹ is A or D;
X² is L or M;
X³ is A or V;
X⁴ is S or N;
X⁵ is H, N, or Y;
X⁶ is P or Q;
X⁷ is A or N;
X⁸ is T or K;
X⁹ is N, L, Q, G, F, V, K, S, R, T, H, or P;
X¹⁰ is F or Y;
X¹¹ is L or H;
X¹² is W, F, Y, P, N, V, K, M, L, G, S.

16. The method of claim 9, wherein the immunoglobulin light chain polypeptide comprises any of SEQ ID NOs: 157-192, or at least the CDRs thereof; or an amino acid sequence with at least 90% sequence identity thereto.

17. The method of claim 9, wherein the BTLA binding agent comprises
(a) a CDRH1 comprising SEQ ID NO: 201;
(b) a CDRH2 comprising SEQ ID NO: 202;
(c) a CDRH3 comprising SEQ ID NO: 203;
(d) a CDRL1 comprising SEQ ID NO: 204;
(e) a CDRL2 comprising SEQ ID NO: 205; and
(f) a CDRL3 comprising SEQ ID NO: 206.

18. The method of claim 9, wherein the BTLA binding agent comprises an immunoglobulin heavy chain variable region of SEQ ID NO: 144, or at least the CDRs thereof; and an immunoglobulin light chain variable region of SEQ ID NO: 174, or at least the CDRs thereof; or comprising an immunoglobulin heavy chain variable region with 90% or more sequence identity to SEQ ID NO: 144, and an immunoglobulin light chain variable region of SEQ ID NO: 174.

19. The method of claim 9, further comprising selecting the subject for treatment with a BTLA binding agent, the method comprising detecting soluble BTLA (sBTLA) in a sample of blood, plasma, serum, or tissue from a subject; and selecting the subject for treatment if the sBTLA concentration in the sample from the subject is lower than the sBTLA level of a normal, non-diseased subject of the same type.

20. The method of claim 19, wherein the method further comprises comparing the concentration of sBTLA in the blood, plasma, serum, or tissue sample to a reference sBTLA concentration.

21. The method of claim 20, wherein the reference sBTLA concentration is the concentration of sBTLA in the blood, plasma, serum, or tissue of another normal, non-diseased subject of the same type, or a reference sBTLA concentration established by the statistical analysis of the sBTLA concentration in the blood, plasma, serum, or tissue of a population of such subjects.

22. (canceled)

23. The method of claim 19, wherein the BTLA binding agent comprises an immunoglobulin heavy chain variable region of SEQ ID NO: 144, or at least the CDRs thereof; and an immunoglobulin heavy light chain variable region of SEQ ID NO: 174, or at least the CDRs thereof; or comprises an immunoglobulin heavy chain variable region with 90% or more sequence identity to SEQ ID NO: 144, and an immunoglobulin light chain variable region with at least 90% or more sequence identity to SEQ ID NO: 174.

24. The method claim 19, wherein the subject has an autoimmune or inflammatory disease.

25. The method of claim 24, wherein the disease is rheumatoid arthritis, graft vs host disease, psoriasis, or inflammatory bowel disease.


SEQUENCE LISTING (234 sequences)

SEQ ID NO: 1 - protein, 118 aa, synthetic construct
  REGION 1..118: note = Synthetic
QVQLVQSGAEVKKPGASVKVSCKASGFTFTDYTIHWVRQAPGQRLEWMGWIYPGSGNTKYNDKFKDRVTITRDTSASTAYMELSSLRSEDTAVYYCARRNYYAMEYWGQGTTVTVSSA

SEQ ID NO: 2 - protein, 118 aa, synthetic construct
  REGION 1..118: note = synthetic
QVQLVQSGAEVKKPGASVKVSCKASGFTFTDYTIHWVRQAPGQRLEWMGWIYPGSGNTKYNDKFKDRVTITADKSASTAYMELSSLRSEDTAVYYCARRNYYAMEYWGQGTTVTVSSA

SEQ ID NO: 3 - protein, 118 aa, synthetic construct
  REGION 1..118: note = synthetic
QVQLVQSGAEVKKPGASVKVSCKASGFTFTDYTIHWVRQAPGQRLEWMGWIYPGSGNTKYNDKFKDRVTITRDTSSTTAYMELSSLRSEDTAVYYCARRNYYAMEYWGQGTTVTVSSA

SEQ ID NO: 4 - protein, 118 aa, synthetic construct
  REGION 1..118: note = synthetic
QVQLVQSGAEVKKPGASVKVSCKASGFTFTDYTIHWVRQAPGQRLEWMGWIYPGSGNTKYNDKFKDRVTITRDTSASTAYMELSSLRSEDTAVYFCARRNYYAMEYWGQGTTVTVSSA

SEQ ID NO: 5 - protein, 118 aa, synthetic construct
  REGION 1..118: note = synthetic
QVQLVQSGAEVKKPGASVKVSCKASGYTLTDYTIHWVRQAPGQRLEWMGWIYPGSGNTKYNDKFKVRVTITRDTSASTAYMELSSLRSEDTAVYYCARRNYYAMEYWGQGTTVTVSSA

SEQ ID NO: 6 - protein, 118 aa, synthetic construct
  REGION 1..118: note = synthetic
QVQLVQSGAEVKKPGASVKVSCKASGFTFTDYTIHWVRQAPGQRLEWMGWIYPGSGNTKYNDKFKDRVTITRDTSASTAYMELSSLRSEDTAVYYCARRSHYAMDYWGQGTTVTVSSA

SEQ ID NO: 7 - protein, 118 aa, synthetic construct
  REGION 1..118: note = synthetic
QVQLVQSGAEVKKPGASVKVSCKASGYTLTDYTIHWVRQAPGQRLEWMGWIYPGSGNTKYNDKFKVRVTITRDTSASTAYMELSSLRSEDTAVYYCARRSHYAMDYWGQGTTVTVSSA

SEQ ID NO: 8 - protein, 118 aa, synthetic construct
  REGION 1..118: note = synthetic
QVQLVQSGAEVKKPGASVKVSCKASGYTLTDYTIHWVRQAPGQRLEWMGWIYPGSGNTKYNDKFKVRVTITADKSASTAYMELSSLRSEDTAVYYCARRNYYAMEYWGQGTTVTVSSA

SEQ ID NO: 9 - protein, 118 aa, synthetic construct
  REGION 1..118: note = synthetic
QVQLVQSGAEVKKPGASVKVSCKASGFTFTDYTIHWVRQAPGQRLEWMGWIYPGSGNTKYNDKFKDRVTITADKSASTAYMELSSLRSEDTAVYYCARRSHYAMDYWGQGTTVTVSSA

SEQ ID NO: 10 - protein, 118 aa, synthetic construct
  REGION 1..118: note = synthetic
QVQLVQSGAEVKKPGASVKVSCKASGYTLTDYTIHWVRQAPGQRLEWMGWIYPGSGNTKYNDKFKVRVTITADKSASTAYMELSSLRSEDTAVYYCARRSHYAMDYWGQGTTVTVSSA

SEQ ID NO: 11 - protein, 118 aa, synthetic construct
  REGION 1..118: note = synthetic
QVQLVQSGAEVKKPGASVKVSCKASGFTFTDYTIHWVRQAPGQRLEWMGWIYPGSGNTKYNDKFKDRVTITADKSSTTAYMELSSLRSEDTAVYYCARRNYYAMEYWGQGTTVTVSSA

SEQ ID NO: 12 - protein, 118 aa, synthetic construct
  REGION 1..118: note = synthetic
QVQLVQSGAEVKKPGASVKVSCKASGFTFTDYTIHWVRQAPGQRLEWMGWIYPGSGNTKYNDKFKDRVTITADKSASTAYMELSSLRSEDTAVYYCARRNYYVMEYWGQGTTVTVSSA

SEQ ID NO: 13 - protein, 118 aa, synthetic construct
  REGION 1..118: note = synthetic
QVQLVQSGAEVKKPGASVKVSCKASGYTLTDYTIHWVRQAPGQRLEWMGWIYPGSGNTKYNDKFKVRVTITRDTSASTAYMELSSLRSEDTAVYYCARRNYYVMEYWGQGTTVTVSSA

SEQ ID NO: 14 - protein, 118 aa, synthetic construct
  REGION 1..118: note = synthetic
QVQLVQSGAEVKKPGASVKVSCKASGFTFTDYTIHWVRQAPGQRLEWMGWIYPGSGNTKYNDKFKDRVTITADTSASTAYMELSSLRSEDTAVYYCARRNYYAMEYWGQGTTVTVSSA

SEQ ID NO: 15 - protein, 118 aa, synthetic construct
  REGION 1..118: note = synthetic
QVQLVQSGAEVKKPGASVKVSCKASGFTFTDYTIHWVRQAPGQRLEWMGWIYPGSGNTKYNDKFKDRVTITRDKSASTAYMELSSLRSEDTAVYYCARRNYYAMEYWGQGTTVTVSSA

SEQ ID NO: 16 - protein, 108 aa, synthetic construct
  REGION 1..108: note = synthetic
DIVMTQSPDSLAVSLGERATINCKASQNVFTNVAWYQQKPGQPPKLLIYSASYRYSGVPDRFSGSGSGTDFTLTISSLQAEDVAVYYCQQYSTYPYTFGQGTKLEIKR

SEQ ID NO: 17 - protein, 108 aa, synthetic construct
  REGION 1..108: note = synthetic
DIVMTQSPDSLAVSLGERATINCKASQNVFTNVAWYQQKPGQSPKPLIYSASYRYSGVPDRFSGSGSGTDFTLTISSLQAEDVAVYYCQQYSTYPYTFGQGTKLEIKR

SEQ ID NO: 18 - protein, 108 aa, synthetic construct
  REGION 1..108: note = synthetic
DIVMTQSPDSLAVSLGERATINCKASQNVFTNVAWYQQKPGQPPKLLIYSASYRYSGVPDRFSGSGSGTDFTLTISSLQAEDVAVYFCQQYSTYPYTFGQGTKLEIKR

SEQ ID NO: 19 - protein, 108 aa, synthetic construct
  REGION 1..108: note = synthetic
DIVMTQSPDSLAVSLGERATINCKASQNVFTNVAWYQQKPGQSPKPLIYSASYRYSGVPDRFSGSGSGTDFTLTISSLQAEDVAVYFCQQYSTYPYTFGQGTKLEIKR

SEQ ID NO: 20 - protein, 108 aa, synthetic construct
  REGION 1..108: note = synthetic
DIVMTQSPDSLAVSLGERATINCKASQNVFTNVAWYQQKPGQSPKPLIYSASYRSSGVPDRFSGSGSGTDFTLTISSLQAEDVAVYYCQQYSTYPYTFGQGTKLEIKR

SEQ ID NO: 21 - protein, 108 aa, synthetic construct
  REGION 1..108: note = synthetic
DIVMTQSPDSLAVSLGERATINCKASQNVFTNVAWYQQKPGQSPKPLIYSASYRSSGVPDRFSGSGSGTDFTLTISSLQAEDVAVYYCQQYNSYPYTFGQGTKLEIKR

SEQ ID NO: 22 - protein, 108 aa, synthetic construct
  REGION 1..108: note = synthetic
DIVMTQSPDSLAVSLGERATINCKASQNVFTNVAWYQQKPGQSPKLLIYSASYRYSGVPDRFSGSGSGTDFTLTISSLQAEDVAVYYCQQYSTYPYTFGQGTKLEIKR

SEQ ID NO: 23 - protein, 108 aa, synthetic construct
  REGION 1..108: note = synthetic
DIVMTQSPDSLAVSLGERATINCKASQNVFTNVAWYQQKPGQPPKPLIYSASYRYSGVPDRFSGSGSGTDFTLTISSLQAEDVAVYYCQQYSTYPYTFGQGTKLEIKR

SEQ ID NO: 24 - protein, 108 aa, synthetic construct
  REGION 1..108: note = synthetic
DIVMTQSPDSLAVSLGERATINCKASQNVFTNVAWYQQKPGQSPKLLIYSASYRYSGVPDRFSGSGSGTDFTLTISSLQAEDVAVYFCQQYSTYPYTFGQGTKLEIKR

SEQ ID NO: 25 - protein, 108 aa, synthetic construct
  REGION 1..108: note = synthetic
DIVMTQSPDSLAVSLGERATINCKASQNVFTNVAWYQQKPGQPPKPLIYSASYRYSGVPDRFSGSGSGTDFTLTISSLQAEDVAVYFCQQYSTYPYTFGQGTKLEIKR

SEQ ID NO: 26 - protein, 118 aa, synthetic construct
  REGION 1..118: note = synthetic
  SITE 27: note = MISC_FEATURE - Xaa is Phe or Tyr
  SITE 29: note = MISC_FEATURE - Xaa is Phe or Leu
  SITE 63: note = MISC_FEATURE - Xaa is Lys or Glu
  SITE 66: note = MISC_FEATURE - Xaa is Asp or Val
  SITE 72: note = MISC_FEATURE - Xaa is Ala or Arg
  SITE 74: note = MISC_FEATURE - Xaa is Lys or Thr
  SITE 76: note = MISC_FEATURE - Xaa is Ala or Ser
  SITE 77: note = MISC_FEATURE - Xaa is Ser or Thr
  SITE 95: note = MISC_FEATURE - Xaa is Tyr or Phe
  SITE 100: note = MISC_FEATURE - Xaa is Asn or Ser
  SITE 101: note = MISC_FEATURE - Xaa is Tyr or His
  SITE 103: note = MISC_FEATURE - Xaa is Ala or Val
  SITE 105: note = MISC_FEATURE - Xaa is Glu or Asp
QVQLVQSGAEVKKPGASVKVSCKASGXTXTDYTIHWVRQAPGQRLEWMGWIYPGSGNTKYNDXFKXRVTITXDXSXXTAYMELSSLRSEDTAVYXCARRXXYXMXYWGQGTTVTVSSA

SEQ ID NO: 27 - protein, 5 aa, synthetic construct
  REGION 1..5: note = synthetic
DYTIH

SEQ ID NO: 28 - protein, 17 aa, synthetic construct
  REGION 1..17: note = synthetic
WIYPGSGNTKYNDKFKD

SEQ ID NO: 29 - protein, 8 aa, synthetic construct
  REGION 1..8: note = synthetic
RNYYAMEY

SEQ ID NO: 30 - protein, 17 aa, synthetic construct
  REGION 1..17: note = synthetic
  SITE 14: note = MISC_FEATURE - Xaa is Lys or Glu
  SITE 17: note = MISC_FEATURE - Xaa is Asp or Val
WIYPGSGNTKYNDXFKX

SEQ ID NO: 31 - protein, 17 aa, synthetic construct
  REGION 1..17: note = synthetic
WIYPGSGNTKYNDKFKV

SEQ ID NO: 32 - protein, 8 aa, synthetic construct
  REGION 1..8: note = synthetic
  SITE 2: note = MISC_FEATURE - Xaa is Asn or Ser
  SITE 3: note = MISC_FEATURE - Xaa is Tyr or His
  SITE 5: note = MISC_FEATURE - Xaa is Ala or Val
  SITE 7: note = MISC_FEATURE - Xaa is Glu or Asp
RXXYXMXY

SEQ ID NO: 33 - protein, 8 aa, synthetic construct
  REGION 1..8: note = synthetic
RSHYAMDY

SEQ ID NO: 34 - protein, 8 aa, synthetic construct
  REGION 1..8: note = synthetic
RNYYVMEY

SEQ ID NO: 35 - protein, 108 aa, synthetic construct
  REGION 1..108: note = synthetic
  SITE 43: note = MISC_FEATURE - Xaa is Ser or Pro
  SITE 46: note = MISC_FEATURE - Xaa is Pro or Leu
  SITE 55: note = MISC_FEATURE - Xaa is Tyr or Ser
  SITE 87: note = MISC_FEATURE - Xaa is Tyr or Phe
  SITE 92: note = MISC_FEATURE - Xaa is Ser or Asn
  SITE 93: note = MISC_FEATURE - Xaa is Thr or Ser
DIVMTQSPDSLAVSLGERATINCKASQNVFTNVAWYQQKPGQXPKXLIYSASYRXSGVPDRFSGSGSGTDFTLTISSLQAEDVAVYXCQQYXXYPYTFGQGTKLEIKR

SEQ ID NO: 36 - protein, 11 aa, synthetic construct
  REGION 1..11: note = synthetic
KASQNVFTNVA

SEQ ID NO: 37 - protein, 7 aa, synthetic construct
  REGION 1..7: note = synthetic
SASYRYS

SEQ ID NO: 38 - protein, 9 aa, synthetic construct
  REGION 1..9: note = synthetic
QQYSTYPYT

SEQ ID NO: 39 - protein, 7 aa, synthetic construct
  REGION 1..7: note = synthetic
  SITE 6: note = MISC_FEATURE - Xaa is Tyr or Ser
SASYRXS

SEQ ID NO: 40 - protein, 7 aa, synthetic construct
  REGION 1..7: note = synthetic
SASYRSS

SEQ ID NO: 41 - protein, 9 aa, synthetic construct
  REGION 1..9: note = synthetic
  SITE 4: note = MISC_FEATURE - Xaa is Ser or Asn
  SITE 5: note = MISC_FEATURE - Xaa is Thr or Ser
QQYXXYPYT

SEQ ID NO: 42 - protein, 9 aa, synthetic construct
  REGION 1..9: note = synthetic
QQYNSYPYT

SEQ ID NO: 43 - protein, 125 aa, synthetic construct
  REGION 1..125: note = synthetic
QVQLVQSGAEVKKPGSSVKVSCKASGYAFSNSWMNWVRQAPGQGLEWMGRIYPGDGDTNYNGKFKGRVTITADKSTSTAYMELSSLRSEDTAVYYCAKSGTFNDGNYRWYFDVWGKGTTVTVSSA

SEQ ID NO: 44 - protein, 125 aa, synthetic construct
  REGION 1..125: note = synthetic
QVQLVQSGAEVKKPGSSVKVSCKASGYAFSNSWMNWVRQAPGQGLEWMGRIYPGDGDTNYNGKFKGRVTITADKSTSTAYMELSSLRSEDTAVYFCAKSGTFNDGNYRWYFDVWGKGTTVTVSSA

SEQ ID NO: 45 - protein, 125 aa, synthetic construct
  REGION 1..125: note = synthetic
QVQLVQSGAEVKKPGSSVKVSCKASGYAFSNSWMNWVRQAPGQGLEWMGRIYPGDGDTNYNGKFKGRVTITADKSTSTAYMELSSLRSEDTAVYYCARSGTFNDGNYRWYFDVWGKGTTVTVSSA

SEQ ID NO: 46 - protein, 125 aa, synthetic construct
  REGION 1..125: note = synthetic
QVQLVQSGAEVKKPGSSVKVSCKASGYAFSNSWMNWVRQAPGQGLEWMGRIYPGDGDTNYNGKFKGRVTITADKSTSTAYMELSSLRSEDTAVYYCARSGTFDDGNYRWYFDVWGKGTTVTVSSA

SEQ ID NO: 47 - protein, 125 aa, synthetic construct
  REGION 1..125: note = synthetic
QVQLVQSGAEVKKPGSSVKVSCKASGYAFSTSWMNWVRQAPGQGLEWMGRIYPGDGDTNYNGKFKGRVTITADKSTSTAYMELSSLRSEDTAVYYCARSGTFNDGNYRWYFDVWGKGTTVTVSSA

SEQ ID NO: 48 - protein, 125 aa, synthetic construct
  REGION 1..125: note = synthetic
QVQLVQSGAEVKKPGSSVKVSCKASGYAFSSSWMNWVRQAPGQGLEWMGRIYPGDGDTNYNGKFKGRVTITADKSTSTAYMELSSLRSEDTAVYYCARSGTFNDGNYRWYFDVWGKGTTVTVSSA

SEQ ID NO: 49 - protein, 125 aa, synthetic construct
  REGION 1..125: note = synthetic
QVQLVQSGAEVKKPGSSVKVSCKASGYAFSTSWMNWVRQAPGQGLEWMGRIYPGDGDTNYNGKFKGRVTITADKSTSTAYMELSSLRSEDTAVYYCARSGTFDDGNYRWYFDVWGKGTTVTVSSA

SEQ ID NO: 50 - protein, 125 aa, synthetic construct
  REGION 1..125: note = synthetic
QVQLVQSGAEVKKPGSSVKVSCKASGYAFSNSWMNWVRQAPGQGLEWMGRIYPGDGDTNYNGKFKGRVTITADKSTSTAYMELSSLRSEDTAVYFCAKSGTFDDGNYRWYFDVWGKGTTVTVSSA

SEQ ID NO: 51 - protein, 125 aa, synthetic construct
  REGION 1..125: note = synthetic
QVQLVQSGAEVKKPGSSVKVSCKASGYAFSTSWMNWVRQAPGQGLEWMGRIYPGDGDTNYNGKFKGRVTITADKSTSTAYMELSSLRSEDTAVYFCAKSGTFNDGNYRWYFDVWGKGTTVTVSSA

SEQ ID NO: 52 - protein, 125 aa, synthetic construct
  REGION 1..125: note = synthetic
QVQLVQSGAEVKKPGSSVKVSCKASGYAFSTSWMNWVRQAPGQGLEWMGRIYPGDGDTNYNGKFKGRVTITADKSTSTAYMELSSLRSEDTAVYFCAKSGTFDDGNYRWYFDVWGKGTTVTVSSA

SEQ ID NO: 53 - protein, 125 aa, synthetic construct
  REGION 1..125: note = synthetic
QVQLVQSGAEVKKPGSSVKVSCKASGYAFSNSWMNWVRQAPGQGLEWMGRIYPGDGDTNYNGKFKGRVTITADKSTNTAYMELSSLRSEDTAVYFCAKSGTFNDGNYRWYFDVWGKGTTVTVSSA

SEQ ID NO: 54 - protein, 125 aa, synthetic construct
  REGION 1..125: note = synthetic
QVQLVQSGAEVKKPGSSVKVSCKASGYAFSNSWMNWVRQAPGQGLEWMGRIYPGDGDTNYNGKFKGRVTITADKSTNTAYMELSSLRSEDTAVYFCAKSGTFNDGNYHWYFDVWGKGTTVTVSSA

SEQ ID NO: 55 - protein, 125 aa, synthetic construct
  REGION 1..125: note = synthetic
QVQLVQSGAEVKKPGSSVKVSCKASGYAFSNSWMNWVRQAPGQGLEWMGRIYPGDGDTNYNGKFKGRVTITADKSTSTAYMELRSLRSEDTAVYYCARSGTFNDGNYRWYFDVWGKGTTVTVSSA

SEQ ID NO: 56 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYNGKFKGRVTITADKSTSTAYMELSSLRSEDTAVYYCARSGTFNDGNYRWYFDVWGKGTTVTVSSA

SEQ ID NO: 57 - protein, 125 aa, synthetic construct
  REGION 1..125: note = synthetic
QVQLVQSGAEVKKPGSSVKVSCKASGYAFSNSWMNWVRQAPGQGLEWMGRIYPGDGDTNYNGKFKGRVTITADKSTTTAYMELSSLRSEDTAVYYCARSGTFNDGNYRWYFDVWGKGTTVTVSSA

SEQ ID NO: 58 - protein, 125 aa, synthetic construct
  REGION 1..125: note = synthetic
QVQLVQSGAEVKKPGSSVKVSCKASGYAFSNSWMNWVRQAPGQGLEWMGRIYPGDGDTNYNGKFKGRVTITADKSTSTAYMELSSLRSEDTAVYFCARSGTFNDGNYRWYFDVWGKGTTVTVSSA

SEQ ID NO: 59 - protein, 125 aa, synthetic construct
  REGION 1..125: note = synthetic
QVQLVQSGAEVKKPGSSVKVSCKASGYAFSNSWMNWVRQAPGQGLEWMGRIYPGDGDTNYNGKFKGRVTITADKSTSTAYMELRSLRSEDTAVYFCAKSGTFNDGNYRWYFDVWGKGTTVTVSSA

SEQ ID NO: 60 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYNGKFKGRVTITADKSTSTAYMELSSLRSEDTAVYFCAKSGTFNDGNYRWYFDVWGKGTTVTVSSA

SEQ ID NO: 61 - protein, 125 aa, synthetic construct
  REGION 1..125: note = synthetic
QVQLVQSGAEVKKPGSSVKVSCKASGYAFSNSWMNWVRQAPGQGLEWMGRIYPGDGDTNYNGKFKGRVTITADKSTTTAYMELSSLRSEDTAVYFCAKSGTFNDGNYRWYFDVWGKGTTVTVSSA

SEQ ID NO: 62 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYNGKFKGRVTITADKSTSTAYMELRSLRSEDTAVYFCAKSGTFNDGNYRWYFDVWGKGTTVTVSSA

SEQ ID NO: 63 - protein, 125 aa, synthetic construct
  REGION 1..125: note = synthetic
QVQLVQSGAEVKKPGSSVKVSCKASGYAFSNSWMNWVRQAPGQGLEWMGRIYPGDGDTNYNGKFKGRVTITADKSTTTAYMELRSLRSEDTAVYFCAKSGTFNDGNYRWYFDVWGKGTTVTVSSA

SEQ ID NO: 64 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYNGKFKGRVTITADKSTTTAYMELRSLRSEDTAVYFCAKSGTFNDGNYRWYFDVWGKGTTVTVSSA

SEQ ID NO: 65 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYNGKFKGRVTITADKSTTTAYMELSSLRSEDTAVYFCAKSGTFNDGNYRWYFDVWGKGTTVTVSSA

SEQ ID NO: 66 - protein, 126 aa, synthetic construct
  REGION 1..126: note = synthetic
QVQLVQSGAEVKKPGSSVKVSCKASGYVFSNSWMNWVRQAPGQGLEWMGRIYPAGDGDTNYNGKFKGRVTITADKSTTTAYMELSSLRSEDTAVYFCAKSGTFNDGNYRWYFDVWGKGTTVTVSSA

SEQ ID NO: 67 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYNGKFKGRVTITADKFTTTAYMELSSLRSEDTAVYFCAKSGTFNDGNYRWYFDVWGKGTTVTVSSA

SEQ ID NO: 68 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYNGKFKGRVTITADKSTTTAYMELSSLRSEDTAVYFCAKSGTFNDGNYRWYFDVWGKGTTVTVSSA

SEQ ID NO: 69 - protein, 125 aa, synthetic construct
  REGION 1..125: note = synthetic
QVQLVQSGAEVKKPGSSVKVSCKASGYVFSNSWMNWVRQAPGQGLEWMGRIYPGTGDTNYNGKFKGRVTITADKSTTTAYMELSSLRSEDTAVYFCAKSGTFNDGNYRWYFDVWGKGTTVTVSSA

SEQ ID NO: 70 - protein, 125 aa, synthetic construct
  REGION 1..125: note = synthetic
QVQLVQSGAEVKKPGSSVKVSCKASGYVFSNSWMNWVRQAPGQGLEWMGRIYPGRGDTNYNGKFKGRVTITADKSTTTAYMELSSLRSEDTAVYFCAKSGTFNDGNYRWYFDVWGKGTTVTVSSA

SEQ ID NO: 71 - protein, 125 aa, synthetic construct
  REGION 1..125: note = synthetic
QVQLVQSGAEVKKPGSSVKVSCKASGYVFSNSWMNWVRQAPGQGLEWMGRIYPGPGDTNYNGKFKGRVTITADKSTTTAYMELSSLRSEDTAVYFCAKSGTFNDGNYRWYFDVWGKGTTVTVSSA

SEQ ID NO: 72 - protein, 125 aa, synthetic construct
  REGION 1..125: note = synthetic
QVQLVQSGAEVKKPGSSVKVSCKASGYVFSNSWMNWVRQAPGQGLEWMGRIYPGSGDTNYNGKFKGRVTITADKSTTTAYMELSSLRSEDTAVYFCAKSGTFNDGNYRWYFDVWGKGTTVTVSSA

SEQ ID NO: 73 - protein, 125 aa, synthetic construct
  REGION 1..125: note = synthetic
QVQLVQSGAEVKKPGSSVKVSCKASGYVFSNSWMNWVRQAPGQGLEWMGRIYPGHGDTNYNGKFKGRVTITADKSTTTAYMELSSLRSEDTAVYFCAKSGTFNDGNYRWYFDVWGKGTTVTVSSA

SEQ ID NO: 74 - protein, 125 aa, synthetic construct
  REGION 1..125: note = synthetic
QVQLVQSGAEVKKPGSSVKVSCKASGYVFSNSWMNWVRQAPGQGLEWMGRIYPGFGDTNYNGKFKGRVTITADKSTTTAYMELSSLRSEDTAVYFCAKSGTFNDGNYRWYFDVWGKGTTVTVSSA

SEQ ID NO: 75 - protein, 125 aa, synthetic construct
  REGION 1..125: note = synthetic
QVQLVQSGAEVKKPGSSVKVSCKASGYVFSNSWMNWVRQAPGQGLEWMGRIYPGLGDTNYNGKFKGRVTITADKSTTTAYMELSSLRSEDTAVYFCAKSGTFNDGNYRWYFDVWGKGTTVTVSSA

SEQ ID NO: 76 - protein, 125 aa, synthetic construct
  REGION 1..125: note = synthetic
QVQLVQSGAEVKKPGSSVKVSCKASGYVFSNSWMNWVRQAPGQGLEWMGRIYPGGGDTNYNGKFKGRVTITADKSTTTAYMELSSLRSEDTAVYFCAKSGTFNDGNYRWYFDVWGKGTTVTVSSA

SEQ ID NO: 77 - protein, 125 aa, synthetic construct
  REGION 1..125: note = synthetic
QVQLVQSGAEVKKPGSSVKVSCKASGYVFSNSWMNWVRQAPGQGLEWMGRIYPGQGDTNYNGKFKGRVTITADKSTTTAYMELSSLRSEDTAVYFCAKSGTFNDGNYRWYFDVWGKGTTVTVSSA

SEQ ID NO: 78 - protein, 125 aa, synthetic construct
  REGION 1..125: note = synthetic
QVQLVQSGAEVKKPGSSVKVSCKASGYVFSNSWMNWVRQAPGQGLEWMGRIYPGYGDTNYNGKFKGRVTITADKSTTTAYMELSSLRSEDTAVYFCAKSGTFNDGNYRWYFDVWGKGTTVTVSSA

SEQ ID NO: 79 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYTGKFKGRVTITADKSTTTAYMELSSLRSEDTAVYFCAKSGTFNDGNYRWYFDVWGKGTTVTVSSA

SEQ ID NO: 80 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYPGKFKGRVTITADKSTTTAYMELSSLRSEDTAVYFCAKSGTFNDGNYRWYFDVWGKGTTVTVSSA

SEQ ID NO: 81 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYGGKFKGRVTITADKSTTTAYMELSSLRSEDTAVYFCAKSGTFNDGNYRWYFDVWGKGTTVTVSSA

SEQ ID NO: 82 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYVGKFKGRVTITADKSTTTAYMELSSLRSEDTAVYFCAKSGTFNDGNYRWYFDVWGKGTTVTVSSA

SEQ ID NO: 83 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYSGKFKGRVTITADKSTTTAYMELSSLRSEDTAVYFCAKSGTFNDGNYRWYFDVWGKGTTVTVSSA

SEQ ID NO: 84 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYLGKFKGRVTITADKSTTTAYMELSSLRSEDTAVYFCAKSGTFNDGNYRWYFDVWGKGTTVTVSSA

SEQ ID NO: 85 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYYGKFKGRVTITADKSTTTAYMELSSLRSEDTAVYFCAKSGTFNDGNYRWYFDVWGKGTTVTVSSA

SEQ ID NO: 86 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYFGKFKGRVTITADKSTTTAYMELSSLRSEDTAVYFCAKSGTFNDGNYRWYFDVWGKGTTVTVSSA

SEQ ID NO: 87 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYAGKFKGRVTITADKSTTTAYMELSSLRSEDTAVYFCAKSGTFNDGNYRWYFDVWGKGTTVTVSSA

SEQ ID NO: 88 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYRGKFKGRVTITADKSTTTAYMELSSLRSEDTAVYFCAKSGTFNDGNYRWYFDVWGKGTTVTVSSA

SEQ ID NO: 89 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYQGKFKGRVTITADKSTTTAYMELSSLRSEDTAVYFCAKSGTFNDGNYRWYFDVWGKGTTVTVSSA

SEQ ID NO: 90 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYNGKFKGRVTITADKSTTTAYMELSSLRSEDTAVYFCAKSGTFNEGNYRWYFDVWGKGTTVTVSSA

SEQ ID NO: 91 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYNGKFKGRVTITADKSTTTAYMELSSLRSEDTAVYFCAKSGTFNLGNYRWYFDVWGKGTTVTVSSA

SEQ ID NO: 92 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYNGKFKGRVTITADKSTTTAYMELSSLRSEDTAVYFCAKSGTFNIGNYRWYFDVWGKGTTVTVSSA

SEQ ID NO: 93 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYNGKFKGRVTITADKSTTTAYMELSSLRSEDTAVYFCAKSGTFNRGNYRWYFDVWGKGTTVTVSSA

SEQ ID NO: 94 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYNGKFKGRVTITADKSTTTAYMELSSLRSEDTAVYFCAKSGTFNTGNYRWYFDVWGKGTTVTVSSA

SEQ ID NO: 95 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYNGKFKGRVTITADKSTTTAYMELSSLRSEDTAVYFCAKSGTFNGGNYRWYFDVWGKGTTVTVSSA

SEQ ID NO: 96 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYNGKFKGRVTITADKSTTTAYMELSSLRSEDTAVYFCAKSGTFNSGNYRWYFDVWGKGTTVTVSSA

SEQ ID NO: 97 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYNGKFKGRVTITADKSTTTAYMELSSLRSEDTAVYFCAKSGTFNVGNYRWYFDVWGKGTTVTVSSA

SEQ ID NO: 98 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYNGKFKGRVTITADKSTTTAYMELSSLRSEDTAVYFCAKSGTFNFGNYRWYFDVWGKGTTVTVSSA

SEQ ID NO: 99 - protein, 125 aa, synthetic construct
  REGION 1..125: note = synthetic
QVQLVQSGAEVKKPGSSVKVSCKASGYVFSNSWMNWVRQAPGQGLEWMGRIYPGDGDTNYNGKFKGRVTITADKSTTTAYMELSSLRSEDTAVYFCAKSGTFNYGNYRWYFDVWGKGTTVTVSSA

SEQ ID NO: 100 - protein, 125 aa, synthetic construct
  REGION 1..125: note = synthetic
QVQLVQSGAEVKKPGSSVKVSCKASGYVFSTSYMNWVRQAPGQGLEWMGRIYPGDGDTNYNGKFKGRVTITADKSTTTAYMELSSLRSEDTAVYFCAKSGTFNDGNYRWYFDVWGKGTTVTVSSA

SEQ ID NO: 101 - protein, 125 aa, synthetic construct
  REGION 1..125: note = synthetic
QVQLVQSGAEVKKPGSSVKVSCKASGYVFSTSNMNWVRQAPGQGLEWMGRIYPGDGDTNYNGKFKGRVTITADKSTTTAYMELSSLRSEDTAVYFCAKSGTFNDGNYRWYFDVWGKGTTVTVSSA

SEQ ID NO: 102 - protein, 125 aa, synthetic construct
  REGION 1..125: note = synthetic
QVQLVQSGAEVKKPGSSVKVSCKASGYVFSTSVMNWVRQAPGQGLEWMGRIYPGDGDTNYNGKFKGRVTITADKSTTTAYMELSSLRSEDTAVYFCAKSGTFNDGNYRWYFDVWGKGTTVTVSSA

SEQ ID NO: 103 - protein, 125 aa, synthetic construct
  REGION 1..125: note = synthetic
QVQLVQSGAEVKKPGSSVKVSCKASGYVFSTSLMNWVRQAPGQGLEWMGRIYPGDGDTNYNGKFKGRVTITADKSTTTAYMELSSLRSEDTAVYFCAKSGTFNDGNYRWYFDVWGKGTTVTVSSA

SEQ ID NO: 104 - protein, 125 aa, synthetic construct
  REGION 1..125: note = synthetic
QVQLVQSGAEVKKPGSSVKVSCKASGYVFSTSSMNWVRQAPGQGLEWMGRIYPGDGDTNYNGKFKGRVTITADKSTTTAYMELSSLRSEDTAVYFCAKSGTFNDGNYRWYFDVWGKGTTVTVSSA

SEQ ID NO: 105 - protein, 125 aa, synthetic construct
  REGION 1..125: note = synthetic
QVQLVQSGAEVKKPGSSVKVSCKASGYVFSTSDMNWVRQAPGQGLEWMGRIYPGDGDTNYNGKFKGRVTITADKSTTTAYMELSSLRSEDTAVYFCAKSGTFNDGNYRWYFDVWGKGTTVTVSSA

SEQ ID NO: 106 - protein, 125 aa, synthetic construct
  REGION 1..125: note = synthetic
QVQLVQSGAEVKKPGSSVKVSCKASGYVFSTSKMNWVRQAPGQGLEWMGRIYPGDGDTNYNGKFKGRVTITADKSTTTAYMELSSLRSEDTAVYFCAKSGTFNDGNYRWYFDVWGKGTTVTVSSA

SEQ ID NO: 107 - protein, 125 aa, synthetic construct
  REGION 1..125: note = synthetic
QVQLVQSGAEVKKPGSSVKVSCKASGYVFSTSRMNWVRQAPGQGLEWMGRIYPGDGDTNYNGKFKGRVTITADKSTTTAYMELSSLRSEDTAVYFCAKSGTFNDGNYRWYFDVWGKGTTVTVSSA

SEQ ID NO: 108 - protein, 125 aa, synthetic construct
  REGION 1..125: note = synthetic
QVQLVQSGAEVKKPGSSVKVSCKASGYVFSTSPMNWVRQAPGQGLEWMGRIYPGDGDTNYNGKFKGRVTITADKSTTTAYMELSSLRSEDTAVYFCAKSGTFNDGNYRWYFDVWGKGTTVTVSSA

SEQ ID NO: 109 - protein, 125 aa, synthetic construct
  REGION 1..125: note = synthetic
QVQLVQSGAEVKKPGSSVKVSCKASGYVFSTSGMNWVRQAPGQGLEWMGRIYPGDGDTNYNGKFKGRVTITADKSTTTAYMELSSLRSEDTAVYFCAKSGTFNDGNYRWYFDVWGKGTTVTVSSA

SEQ ID NO: 110 - protein, 125 aa, synthetic construct
  REGION 1..125: note = synthetic
QVQLVQSGAEVKKPGSSVKVSCKASGYVFSTSHMNWVRQAPGQGLEWMGRIYPGDGDTNYNGKFKGRVTITADKSTTTAYMELSSLRSEDTAVYFCAKSGTFNDGNYRWYFDVWGKGTTVTVSSA

SEQ ID NO: 111 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYQGKFKGRVTITADKSTTTAYMELSSLRSEDTAVYFCAKSGTFNDGNYRWYFDVWGKGTTVTVSSA

SEQ ID NO: 112 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYVGKFKGRVTITADKSTTTAYMELSSLRSEDTAVYFCAKSGTFNDGNYRWYFDVWGKGTTVTVSSA

SEQ ID NO: 113 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYNGKFKGRVTITADKSTTTAYMELSSLRSEDTAVYFCAKSGTFNFGNYRWYFDVWGKGTTVTVSSA

SEQ ID NO: 114 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYNGKFKGRVTITADKSTTTAYMELSSLRSEDTAVYFCAKSGTFNSGNYRWYFDVWGKGTTVTVSSA

SEQ ID NO: 115 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYQGKFKGRVTITADKSTTTAYMELSSLRSEDTAVYFCAKSGTFNFGNYRWYFDVWGKGTTVTVSSA

SEQ ID NO: 116 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYQGKFKGRVTITADKSTTTAYMELSSLRSEDTAVYFCAKSGTFNSGNYRWYFDVWGKGTTVTVSSA

SEQ ID NO: 117 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYVGKFKGRVTITADKSTTTAYMELSSLRSEDTAVYFCAKSGTFNFGNYRWYFDVWGKGTTVTVSSA

SEQ ID NO: 118 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYVGKFKGRVTITADKSTTTAYMELSSLRSEDTAVYFCAKSGTFNSGNYRWYFDVWGKGTTVTVSSA

SEQ ID NO: 119 - protein, 125 aa, synthetic construct
  REGION 1..125: note = synthetic
QVQLVQSGAEVKKPGSSVKVSCKASGYVFSTSWMNWVRQAPGQGLEWMGRIYPGDRDTNYNGKFKGRVTITADKSTTTAYMELSSLRSEDTAVYFCAKSGTFNDGNYRWYFDVWGKGTTVTVSSA

SEQ ID NO: 120 - protein, 125 aa, synthetic construct
  REGION 1..125: note = synthetic
QVQLVQSGAEVKKPGSSVKVSCKASGYVFSTSWMNWVRQAPGQGLEWMGRIYPGDKDTNYNGKFKGRVTITADKSTTTAYMELSSLRSEDTAVYFCAKSGTFNDGNYRWYFDVWGKGTTVTVSSA

SEQ ID NO: 121 - protein, 125 aa, synthetic construct
  REGION 1..125: note = synthetic
QVQLVQSGAEVKKPGSSVKVSCKASGYVFSTSWMNWVRQAPGQGLEWMGRIYPGDEDTNYNGKFKGRVTITADKSTTTAYMELSSLRSEDTAVYFCAKSGTFNDGNYRWYFDVWGKGTTVTVSSA

SEQ ID NO: 122 - protein, 125 aa, synthetic construct
  REGION 1..125: note = synthetic
QVQLVQSGAEVKKPGSSVKVSCKASGYVFSTSWMNWVRQAPGQGLEWMGRIYPGDTDTNYNGKFKGRVTITADKSTTTAYMELSSLRSEDTAVYFCAKSGTFNDGNYRWYFDVWGKGTTVTVSSA

SEQ ID NO: 123 - protein, 125 aa, synthetic construct
  REGION 1..125: note = synthetic
QVQLVQSGAEVKKPGSSVKVSCKASGYVFSTSWMNWVRQAPGQGLEWMGRIYPGDVDTNYNGKFKGRVTITADKSTTTAYMELSSLRSEDTAVYFCAKSGTFNDGNYRWYFDVWGKGTTVTVSSA

SEQ ID NO: 124 - protein, 125 aa, synthetic construct
  REGION 1..125: note = synthetic
QVQLVQSGAEVKKPGSSVKVSCKASGYVFSTSWMNWVRQAPGQGLEWMGRIYPGDADTNYNGKFKGRVTITADKSTTTAYMELSSLRSEDTAVYFCAKSGTFNDGNYRWYFDVWGKGTTVTVSSA

SEQ ID NO: 125 - protein, 125 aa, synthetic construct
  REGION 1..125: note = synthetic
QVQLVQSGAEVKKPGSSVKVSCKASGYVFSTSWMNWVRQAPGQGLEWMGRIYPGDDDTNYNGKFKGRVTITADKSTTTAYMELSSLRSEDTAVYFCAKSGTFNDGNYRWYFDVWGKGTTVTVSSA

SEQ ID NO: 126 - protein, 125 aa, synthetic construct
  REGION 1..125: note = synthetic
QVQLVQSGAEVKKPGSSVKVSCKASGYVFSTSWMNWVRQAPGQGLEWMGRIYPGDSDTNYNGKFKGRVTITADKSTTTAYMELSSLRSEDTAVYFCAKSGTFNDGNYRWYFDVWGKGTTVTVSSA

SEQ ID NO: 127 - protein, 125 aa, synthetic construct
  REGION 1..125: note = synthetic
QVQLVQSGAEVKKPGSSVKVSCKASGYVFSTSWMNWVRQAPGQGLEWMGRIYPGDFDTNYNGKFKGRVTITADKSTTTAYMELSSLRSEDTAVYFCAKSGTFNDGNYRWYFDVWGKGTTVTVSSA

SEQ ID NO: 128 - protein, 125 aa, synthetic construct
  REGION 1..125: note = synthetic
QVQLVQSGAEVKKPGSSVKVSCKASGYVFSTSWMNWVRQAPGQGLEWMGRIYPGDYDTNYNGKFKGRVTITADKSTTTAYMELSSLRSEDTAVYFCAKSGTFNDGNYRWYFDVWGKGTTVTVSSA

SEQ ID NO: 129 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYNGKFKGRVTITADKSTTTAYMELSSLRSEDTAVYFCAKSGTFNDGNYRKYFDVWGKGTTVTVSSA

SEQ ID NO: 130 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYNGKFKGRVTITADKSTTTAYMELSSLRSEDTAVYFCAKSGTFNDGNYRRYFDVWGKGTTVTVSSA

SEQ ID NO: 131 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYNGKFKGRVTITADKSTTTAYMELSSLRSEDTAVYFCAKSGTFNDGNYRGYFDVWGKGTTVTVSSA

SEQ ID NO: 132 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYNGKFKGRVTITADKSTTTAYMELSSLRSEDTAVYFCAKSGTFNDGNYRSYFDVWGKGTTVTVSSA

SEQ ID NO: 133 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYNGKFKGRVTITADKSTTTAYMELSSLRSEDTAVYFCAKSGTFNDGNYRAYFDVWGKGTTVTVSSA

SEQ ID NO: 134 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYNGKFKGRVTITADKSTTTAYMELSSLRSEDTAVYFCAKSGTFNDGNYRVYFDVWGKGTTVTVSSA

SEQ ID NO: 135 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYNGKFKGRVTITADKSTTTAYMELSSLRSEDTAVYFCAKSGTFNDGNYRIYFDVWGKGTTVTVSSA

SEQ ID NO: 136 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYNGKFKGRVTITADKSTTTAYMELSSLRSEDTAVYFCAKSGTFNDGNYRPYFDVWGKGTTVTVSSA

SEQ ID NO: 137 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYNGKFKGRVTITADKSTTTAYMELSSLRSEDTAVYFCAKSGTFNDGNYRYYFDVWGKGTTVTVSSA

SEQ ID NO: 138 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYNGKFKGRVTITADKSTTTAYMELSSLRSEDTAVYFCAKSGTFNDGNYRNYFDVWGKGTTVTVSSA

SEQ ID NO: 139 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYNGKFKGRVTITADKSTTTAYMELSSLRSEDTAVYFCAKSGTFNDGNYRLYFDVWGKGTTVTVSSA

SEQ ID NO: 140 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYNGKFKGRVTITADKSTTTAYMELSSLRSEVTAVYFCAKSGTFNDGNYRWYFDVWGKGTTVTVSSA

SEQ ID NO: 141 - protein, 126 aa, synthetic construct
  REGION 1..126: note = synthetic
QVQLVQSGAEVKKPGSSVKVSCKASGYVFSTSWMNWVRQAPGQGLEWMGRIYPGDGDTNYNGKFKGRVTITADKSTTTAYMELSSLRSEDTAVYYFCAKSGTFNDGNYRWYFDVWGKGTTVTVSSA

SEQ ID NO: 142 - protein, 125 aa, synthetic construct
  REGION 1..125: note = synthetic
QVQLVQSGAEVKKPGSSVKVSCKASGYVFSTSFMNWVRQAPGQGLEWMGRIYPGDGDTNYNGKFKGRVTITADKSTTTAYMELSSLRSEDTAVYFCAKSGTFNDGNYRWYFDVWGKGTTVTVSSA

SEQ ID NO: 143 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYNGKFKGRVTITADKSTTTAYMELSSLRSEDTAVYFCAKSGTFNDGNYRFYFDVWGKGTTVTVSSA

SEQ ID NO: 144 - protein, 125 aa, synthetic construct
  REGION 1..125: note = synthetic
QVQLVQSGAEVKKPGSSVKVSCKASGYVFSTSWMNWVRQAPGQGLEWMGRIYPGDADTNYVGKFKGRVTITADKSTTTAYMELSSLRSEDTAVYFCAKSGTFNFGNYRWYFDVWGKGTTVTVSSA

SEQ ID NO: 145 - protein, 125 aa, synthetic construct
  REGION 1..125: note = synthetic
QVQLVQSGAEVKKPGSSVKVSCKASGYVFSTSWMNWVRQAPGQGLEWMGRIYPGDGDTNYVGKFKGRVTITADKSTTTAYMELSSLRSEDTAVYFCAKSGTFNFGNYRRYFDVWGKGTTVTVSSA

SEQ ID NO: 146 - protein, 125 aa, synthetic construct
  REGION 1..125: note = synthetic
QVQLVQSGAEVKKPGSSVKVSCKASGYVFSTSWMNWVRQAPGQGLEWMGRIYPGDADTNYVGKFKGRVTITADKSTTTAYMELSSLRSEDTAVYFCAKSGTFNFGNYRRYFDVWGKGTTVTVSSA

SEQ ID NO: 147 - protein, 125 aa, synthetic construct
  REGION 1..125: note = synthetic
QVQLVQSGAEVKKPGSSVKVSCKASGYVFSTSWMNWVRQAPGQGLEWMGRIYPGDYDTNYVGKFKGRVTITADKSTTTAYMELSSLRSEDTAVYFCAKSGTFNSGNYRWYFDVWGKGTTVTVSSA

SEQ ID NO: 148 - protein, 125 aa, synthetic construct
  REGION 1..125: note = synthetic
QVQLVQSGAEVKKPGSSVKVSCKASGYVFSTSFMNWVRQAPGQGLEWMGRIYPGDYDTNYVGKFKGRVTITADKSTTTAYMELSSLRSEDTAVYFCAKSGTFNSGNYRWYFDVWGKGTTVTVSSA

SEQ ID NO: 149 - protein, 125 aa, synthetic construct
  REGION 1..125: note = synthetic
QVQLVQSGAEVKKPGSSVKVSCKASGYVFSTSFMNWVRQAPGQGLEWMGRIYPGDADTNYVGKFKGRVTITADKSTTTAYMELSSLRSEDTAVYFCAKSGTFNFGNYRWYFDVWGKGTTVTVSSA

SEQ ID NO: 150 - protein, 126 aa, synthetic construct
  REGION 1..126: note = synthetic
QVQLVQSGAEVKKPGSSVKVSCKASGYVFSTSWMNWVRQAPGQGLEWMGRIYPGDGDTNYNGKFKGRVTITADKSTTTAYMELSSLRSEVTAVYYFCAKSGTFNDGNYRWYFDVWGKGTTVTVSSA

SEQ ID NO: 151 - protein, 125 aa, synthetic construct
  REGION 1..125: note = synthetic
QVQLVQSGAEVKKPGSSVKVSCKASGYVFSTSWMNWVRQAPGQGLEWMGRIYPGDADTNYVGKFKGRVTITADKSTTTAYMELSSLRSEVTAVYFCAKSGTFNFGNYRWYFDVWGKGTTVTVSSA

SEQ ID NO: 152 - protein, 126 aa, synthetic construct
  REGION 1..126: note = synthetic
QVQLVQSGAEVKKPGSSVKVSCKASGYVFSTSWMNWVRQAPGQGLEWMGRIYPGDADTNYVGKFKGRVTITADKSTTTAYMELSSLRSEDTAVYYFCAKSGTFNFGNYRWYFDVWGKGTTVTVSSA

SEQ ID NO: 153 - protein, 126 aa, synthetic construct
  REGION 1..126: note = synthetic
QVQLVQSGAEVKKPGSSVKVSCKASGYVFSTSWMNWVRQAPGQGLEWMGRIYPGDADTNYVGKFKGRVTITADKSTTTAYMELSSLRSEVTAVYYFCAKSGTFNFGNYRWYFDVWGKGTTVTVSSA

SEQ ID NO: 154 - protein, 125 aa, synthetic construct
  REGION 1..125: note = synthetic
QVQLVQSGAEVKKPGSSVKVSCKASGYAFSTSWMNWVRQAPGQGLEWMGRIYPGDADTNYVGKFKGRVTITADKSTSTAYMELSSLRSEVTAVYFCAKSGTFNYGNYRWYFDVWGKGTTVTVSSA

SEQ ID NO: 155 - protein, 126 aa, synthetic construct
  REGION 1..126: note = synthetic
QVQLVQSGAEVKKPGSSVKVSCKASGYAFSTSWMNWVRQAPGQGLEWMGRIYPGDADTNYVGKFKGRVTITADKSTSTAYMELSSLRSEDTAVYYFCAKSGTFNYGNYRWYFDVWGKGTTVTVSSA

SEQ ID NO: 156 - protein, 126 aa, synthetic construct
  REGION 1..126: note = synthetic
QVQLVQSGAEVKKPGSSVKVSCKASGYAFSTSWMNWVRQAPGQGLEWMGRIYPGDADTNYVGKFKGRVTITADKSTSTAYMELSSLRSEVTAVYYFCAKSGTFNYGNYRWYFDVWGKGTTVTVSSA

SEQ ID NO: 157 - protein, 108 aa, synthetic construct
  REGION 1..108: note = synthetic
AIQLTQSPSSLSASVGDRVTITCRASENIYSHLAWYQQKPGKAPKLLIYAATNLANGVPSRFSGSGSGTDFTLTISSLQPEDFATYYCQLFWGPPLTFGGGTKVEIKR

SEQ ID NO: 158 - protein, 108 aa, synthetic construct
  REGION 1..108: note = synthetic
DIQMTQSPSSLSASVGDRVTITCRASENIYSHLAWYQQKPGKAPKLLIYAATNLANGVPSRFSGSGSGTDFTLTISSLQPEDFATYYCQLFWGPPLTFGGGTKVEIKR

SEQ ID NO: 159 - protein, 108 aa, synthetic construct
  REGION 1..108: note = synthetic
AIQLTQSPSSLSASVGDRVTITCRASENIYSHLAWYQQKQGKAPKLLIYAATNLANGVPSRFSGSGSGTDFTLTISSLQPEDFATYYCQLFWGPPLTFGGGTKVEIKR

SEQ ID NO: 160 - protein, 108 aa, synthetic construct
  REGION 1..108: note = synthetic
AIQLTQSPSSLSASVGDRVTITCRASENIYSHLAWYQQKPGKAPKLLIYAATNLANGVPSRFSGSGSGTDYTLTISSLQPEDFATYYCQLFWGPPLTFGGGTKVEIKR

SEQ ID NO: 161 - protein, 108 aa, synthetic construct
  REGION 1..108: note = synthetic
AIQLTQSPSSLSASVGDRVTITCRASENIYSHLAWYQQKPGKAPKLLIYAATNLANGVPSRFSGSGSGTDYTLTISSLQPEDFATYYCQHFWGPPLTFGGGTKVEIKR

SEQ ID NO: 162 - protein, 108 aa, synthetic construct
  REGION 1..108: note = synthetic
AIQLTQSPSSLSASVGDRVTITCRASENIYSHLAWYQQKPGKAPKLLIYNAKNLANGVPSRFSGSGSGTDYTLTISSLQPEDFATYYCQLFWGPPLTFGGGTKVEIKR

SEQ ID NO: 163 - protein, 108 aa, synthetic construct
  REGION 1..108: note = synthetic
AIQLTQSPSSLSASVGDRVTITCRASENIYSNLAWYQQKPGKAPKLLIYNAKNLANGVPSRFSGSGSGTDYTLTISSLQPEDFATYYCQLFWGPPLTFGGGTKVEIKR

SEQ ID NO: 164 - protein, 108 aa, synthetic construct
  REGION 1..108: note = synthetic
AIQLTQSPSSLSASVGDRVTITCRASENIYSYLAWYQQKPGKAPKLLIYAATNLANGVPSRFSGSGSGTDYTLTISSLQPEDFATYYCQLFWGPPLTFGGGTKVEIKR

SEQ ID NO: 165 - protein, 108 aa, synthetic construct
  REGION 1..108: note = synthetic
AIQLTQSPSSLSASVGDRVTITCRASENIYSNLAWYQQKPGKAPKLLIYAATNLANGVPSRFSGSGSGTDYTLTISSLQPEDFATYYCQLFWGPPLTFGGGTKVEIKR

SEQ ID NO: 166 - protein, 108 aa, synthetic construct
  REGION 1..108: note = synthetic
AIQLTQSPSSLSASVGDRVTITCRASENIYSYLAWYQQKPGKAPKLLIYAATNLANGVPSRFSGSGSGTDYTLTISSLQPEDFATYYCQHFWGPPLTFGGGTKVEIKR

SEQ ID NO: 167 - protein, 108 aa, synthetic construct
  REGION 1..108: note = synthetic
AIQLTQSPSSLSASVGDRVTITCRASENIYSNLAWYQQKPGKAPKLLIYAATNLANGVPSRFSGSGSGTDYTLTISSLQPEDFATYYCQHFWGPPLTFGGGTKVEIKR

SEQ ID NO: 168 - protein, 108 aa, synthetic construct
  REGION 1..108: note = synthetic
AIQLTQSPSSLSASVGDRVTITCRVSENIYSHLAWYQQKPGKAPKLLIYAATNLANGVPSRFSGSGSGTDYTLTISSLQPEDFATYYCQLFWGPPLTFGGGTKVEIKR

SEQ ID NO: 169 - protein, 108 aa, synthetic construct
  REGION 1..108: note = synthetic
AIQLTQSPSSLSASVGDRVTITCRVSENIYSHLAWYQQKPGKAPKLLIYAATNLAPGVPSRFSGSGSGTDYTLTISSLQPEDFATYYCQLFWGPPLTFGGGTKVEIKR

SEQ ID NO: 170 - protein, 108 aa, synthetic construct
  REGION 1..108: note = synthetic
AIQLTQSPSSLSASVGDRVTITCRVSENIYSHLAWYQQKPGKAPKLLIYAATNLAHGVPSRFSGSGSGTDYTLTISSLQPEDFATYYCQLFWGPPLTFGGGTKVEIKR

SEQ ID NO: 171 - protein, 108 aa, synthetic construct
  REGION 1..108: note = synthetic
AIQLTQSPSSLSASVGDRVTITCRVSENIYSHLAWYQQKPGKAPKLLIYAATNLATGVPSRFSGSGSGTDYTLTISSLQPEDFATYYCQLFWGPPLTFGGGTKVEIKR

SEQ ID NO: 172 - protein, 108 aa, synthetic construct
  REGION 1..108: note = synthetic
AIQLTQSPSSLSASVGDRVTITCRVSENIYSHLAWYQQKPGKAPKLLIYAATNLARGVPSRFSGSGSGTDYTLTISSLQPEDFATYYCQLFWGPPLTFGGGTKVEIKR

SEQ ID NO: 173 - protein, 108 aa, synthetic construct
  REGION 1..108: note = synthetic
AIQLTQSPSSLSASVGDRVTITCRVSENIYSHLAWYQQKPGKAPKLLIYAATNLASGVPSRFSGSGSGTDYTLTISSLQPEDFATYYCQLFWGPPLTFGGGTKVEIKR

SEQ ID NO: 174 - protein, 108 aa, synthetic construct
  REGION 1..108: note = synthetic
AIQLTQSPSSLSASVGDRVTITCRVSENIYSHLAWYQQKPGKAPKLLIYAATNLALGVPSRFSGSGSGTDYTLTISSLQPEDFATYYCQLFWGPPLTFGGGTKVEIKR

SEQ ID NO: 175 - protein, 108 aa, synthetic construct
  REGION 1..108: note = synthetic
AIQLTQSPSSLSASVGDRVTITCRVSENIYSHLAWYQQKPGKAPKLLIYAATNLAKGVPSRFSGSGSGTDYTLTISSLQPEDFATYYCQLFWGPPLTFGGGTKVEIKR

SEQ ID NO: 176 - protein, 108 aa, synthetic construct
  REGION 1..108: note = synthetic
AIQLTQSPSSLSASVGDRVTITCRVSENIYSHLAWYQQKPGKAPKLLIYAATNLAVGVPSRFSGSGSGTDYTLTISSLQPEDFATYYCQLFWGPPLTFGGGTKVEIKR

SEQ ID NO: 177 - protein, 108 aa, synthetic construct
  REGION 1..108: note = synthetic
AIQLTQSPSSLSASVGDRVTITCRVSENIYSHLAWYQQKPGKAPKLLIYAATNLAFGVPSRFSGSGSGTDYTLTISSLQPEDFATYYCQLFWGPPLTFGGGTKVEIKR

SEQ ID NO: 178 - protein, 108 aa, synthetic construct
  REGION 1..108: note = synthetic
AIQLTQSPSSLSASVGDRVTITCRVSENIYSHLAWYQQKPGKAPKLLIYAATNLAGGVPSRFSGSGSGTDYTLTISSLQPEDFATYYCQLFWGPPLTFGGGTKVEIKR

SEQ ID NO: 179 - protein, 108 aa, synthetic construct
  REGION 1..108: note = synthetic
AIQLTQSPSSLSASVGDRVTITCRVSENIYSHLAWYQQKPGKAPKLLIYAATNLAQGVPSRFSGSGSGTDYTLTISSLQPEDFATYYCQLFWGPPLTFGGGTKVEIKR

SEQ ID NO: 180 - protein, 108 aa, synthetic construct
  REGION 1..108: note = synthetic
AIQLTQSPSSLSASVGDRVTITCRVSENIYSHLAWYQQKPGKAPKLLIYAATNLANGVPSRFSGSGSGTDYTLTISSLQPEDFATYYCQLFSGPPLTFGGGTKVEIKR

SEQ ID NO: 181 - protein, 108 aa, synthetic construct
  REGION 1..108: note = synthetic
AIQLTQSPSSLSASVGDRVTITCRVSENIYSHLAWYQQKPGKAPKLLIYAATNLANGVPSRFSGSGSGTDYTLTISSLQPEDFATYYCQLFGGPPLTFGGGTKVEIKR

SEQ ID NO: 182 - protein, 108 aa, synthetic construct
  REGION 1..108: note = synthetic
AIQLTQSPSSLSASVGDRVTITCRVSENIYSHLAWYQQKPGKAPKLLIYAATNLANGVPSRFSGSGSGTDYTLTISSLQPEDFATYYCQLFLGPPLTFGGGTKVEIKR

SEQ ID NO: 183 - protein, 108 aa, synthetic construct
  REGION 1..108: note = synthetic
AIQLTQSPSSLSASVGDRVTITCRVSENIYSHLAWYQQKPGKAPKLLIYAATNLANGVPSRFSGSGSGTDYTLTISSLQPEDFATYYCQLFMGPPLTFGGGTKVEIKR

SEQ ID NO: 184 - protein, 108 aa, synthetic construct
  REGION 1..108: note = synthetic
AIQLTQSPSSLSASVGDRVTITCRVSENIYSHLAWYQQKPGKAPKLLIYAATNLANGVPSRFSGSGSGTDYTLTISSLQPEDFATYYCQLFKGPPLTFGGGTKVEIKR

SEQ ID NO: 185 - protein, 108 aa, synthetic construct
  REGION 1..108: note = synthetic
AIQLTQSPSSLSASVGDRVTITCRVSENIYSHLAWYQQKPGKAPKLLIYAATNLANGVPSRFSGSGSGTDYTLTISSLQPEDFATYYCQLFVGPPLTFGGGTKVEIKR

SEQ ID NO: 186 - protein, 108 aa, synthetic construct
  REGION 1..108: note = synthetic
AIQLTQSPSSLSASVGDRVTITCRVSENIYSHLAWYQQKPGKAPKLLIYAATNLANGVPSRFSGSGSGTDYTLTISSLQPEDFATYYCQLFNGPPLTFGGGTKVEIKR

SEQ ID NO: 187 - protein, 108 aa, synthetic construct
  REGION 1..108: note = synthetic
AIQLTQSPSSLSASVGDRVTITCRVSENIYSHLAWYQQKPGKAPKLLIYAATNLANGVPSRFSGSGSGTDYTLTISSLQPEDFATYYCQLFPGPPLTFGGGTKVEIKR

SEQ ID NO: 188 - protein, 108 aa, synthetic construct
  REGION 1..108: note = synthetic
AIQLTQSPSSLSASVGDRVTITCRVSENIYSHLAWYQQKPGKAPKLLIYAATNLALGVPSRFSGSGSGTDYTLTISSLQPEDFATYYCQLFYGPPLTFGGGTKVEIKR

SEQ ID NO: 189 - protein, 108 aa, synthetic construct
  REGION 1..108: note = synthetic
AIQLTQSPSSLSASVGDRVTITCRVSENIYSHLAWYQQKPGKAPKLLIYAATNLALGVPSRFSGSGSGTDYTLTISSLQPEDFATYYCQLFFGPPLTFGGGTKVEIKR

SEQ ID NO: 190 - protein, 108 aa, synthetic construct
  REGION 1..108: note = synthetic
AIQLTQSPSSLSASVGDRVTITCRVSENIYNHLAWYQQKPGKAPKLLIYAATNLALGVPSRFSGSGSGTDYTLTISSLQPEDFATYYCQLFWGPPLTFGGGTKVEIKR

SEQ ID NO: 191 - protein, 108 aa, synthetic construct
  REGION 1..108: note = synthetic
AIQLTQSPSSLSASVGDRVTITCRVSENIYNHLAWYQQKPGKAPKLLIYAATNLALGVPSRFSGSGSGTDYTLTISSLQPEDFATYYCQLFFGPPLTFGGGTKVEIKR

SEQ ID NO: 192 - protein, 108 aa, synthetic construct
  REGION 1..108: note = synthetic
AIQLTQSPSSLSASVGDRVTITCRASENIYSHLAWYQQKPGKAPKLLIYAATNLALGVPSRFSGSGSGTDYTLTISSLQPEDFATYYCQLFWGPPLTFGGGTKVEIKR

SEQ ID NO: 193 - protein, 127 aa, synthetic construct
  REGION 1..127: note = synthetic
  SITE 28: note = MISC_FEATURE - Xaa is Ala or Val
  SITE 31: note = MISC_FEATURE - Xaa is Asn or Thr
  SITE 33: note = MISC_FEATURE - Xaa is Trp, Phe, His, Gly, Pro, Arg, Lys, Asp, Ser, Leu, Val, Asn, or Tyr
  SITE 54: note = MISC_FEATURE - Xaa is absent or Ala
  SITE 56: note = MISC_FEATURE - Xaa is Asp, Tyr, Gln, Gly, Leu, Phe, His, Ser, Pro, Arg or Thr
  SITE 57: note = MISC_FEATURE - Xaa is Gly, Tyr, Ala, Phe, Ser, Asp, Val, Thr, Glu, Lys or Arg
  SITE 62: note = MISC_FEATURE - Xaa is Asn, Val, Gln, Arg, Ala, Phe, Tyr, Ser, Gly, Pro or Thr
  SITE 76: note = MISC_FEATURE - Xaa is Ser or Phe
  SITE 78: note = MISC_FEATURE - Xaa is Ser, Thr or Asn
  SITE 85: note = MISC_FEATURE - Xaa is Ser or Arg
  SITE 91: note = MISC_FEATURE - Xaa is Asp or Val
  SITE 95: note = MISC_FEATURE - Xaa is absent or Tyr
  SITE 97: note = MISC_FEATURE - Xaa is Tyr or Phe
  SITE 100: note = MISC_FEATURE - Xaa is Lys or Arg
  SITE 105: note = MISC_FEATURE - Xaa is Asn or Asp
  SITE 106: note = MISC_FEATURE - Xaa is Asp, Ser, Phe, Tyr, Val, Gly, Thr, Arg, Ile, Leu or Glu
  SITE 110: note = MISC_FEATURE - Xaa is Arg or His
  SITE 111: note = MISC_FEATURE - Xaa is Trp, Arg, Phe, Leu, Asn, Tyr, Pro, Ile, Val, Ala, Ser, Gly, Arg or Lys
QVQLVQSGAEVKKPGSSVKVSCKASGYXFSXSXMNWVRQAPGQGLEWMGRIYPXGXXDTNYXGKFKGRVTITADKXTXTAYMELXSLRSEXTAVXYXCAXSGTFXXGNYXXYFDVWGKGTTVTVSSA

SEQ ID NO: 194 - protein, 108 aa, synthetic construct
  REGION 1..108: note = synthetic
  SITE 1: note = MISC_FEATURE - Xaa is Ala or Asp
  SITE 4: note = MISC_FEATURE - Xaa is Leu or Met
  SITE 25: note = MISC_FEATURE - Xaa is Ala or Val
  SITE 31: note = MISC_FEATURE - Xaa is Ser or Asn
  SITE 32: note = MISC_FEATURE - Xaa is His, Asn or Tyr
  SITE 40: note = MISC_FEATURE - Xaa is Pro or Gln
  SITE 50: note = MISC_FEATURE - Xaa is Ala or Asn
  SITE 52: note = MISC_FEATURE - Xaa is Thr or Lys
  SITE 56: note = MISC_FEATURE - Xaa is Asn, Leu, Gln, Gly, Phe, Val, Lys, Ser, Arg, Thr, His or Pro
  SITE 71: note = MISC_FEATURE - Xaa is Phe or Tyr
  SITE 90: note = MISC_FEATURE - Xaa is Leu or His
  SITE 92: note = MISC_FEATURE - Xaa is Trp, Phe, Tyr, Pro, Asn, Val, Lys, Met, Leu, Gly or Ser
XIQXTQSPSSLSASVGDRVTITCRXSENIYXXLAWYQQKXGKAPKLLIYXAXNLAXGVPSRFSGSGSGTDXTLTISSLQPEDFATYYCQXFXGPPLTFGGGTKVEIKR

SEQ ID NO: 195 - skipped

SEQ ID NO: 196 - protein, 17 aa, synthetic construct
  REGION 1..17: note = synthetic
  SITE 5: note = MISC_FEATURE - Xaa is absent or Ala
  SITE 7: note = MISC_FEATURE - Xaa is Asp, Tyr, Gln, Gly, Leu, Phe, His, Ser, Pro, Arg or Thr
  SITE 8: note = MISC_FEATURE - Xaa is Gly, Tyr, Ala, Phe, Ser, Asp, Val, Thr, Glu, Lys, or Arg
  SITE 13: note = MISC_FEATURE - Xaa is Asn, Val, Gln, Arg, Ala, Phe, Tyr, Ser, Gly, Pro or Thr
RIYPXGXXDTNYXGKFK

SEQ ID NO: 197 - protein, 16 aa, synthetic construct
  REGION 1..16: note = synthetic
  SITE 1: note = MISC_FEATURE - Xaa is Lys or Arg
  SITE 6: note = MISC_FEATURE - Xaa is Asn or Asp
  SITE 7: note = MISC_FEATURE - Xaa is Asp, Ser, Phe, Tyr, Phe, Val, Ser, Gly, Thr, Arg, Ile, Leu or Glu
  SITE 11: note = MISC_FEATURE - Xaa is Arg or His
  SITE 12: note = MISC_FEATURE - Xaa is Trp, Arg, Phe, Leu, Asn, Tyr, Pro, Ile, Val, Ala, Ser, Gly, Arg or Lys
XSGTFXXGNYXXYFDV

SEQ ID NO: 198 - protein, 11 aa, synthetic construct
  REGION 1..11: note = synthetic
  SITE 2: note = MISC_FEATURE - Xaa is Ala or Val
  SITE 8: note = MISC_FEATURE - Xaa is Ser or Asn
  SITE 9: note = MISC_FEATURE - Xaa is His, Asn or Tyr
RXSENIYXXLA

SEQ ID NO: 199 - protein, 7 aa, synthetic construct
  REGION 1..7: note = synthetic
  SITE 1: note = MISC_FEATURE - Xaa is Ala or Asn
  SITE 3: note = MISC_FEATURE - Xaa is Thr or Lys
  SITE 7: note = MISC_FEATURE - Xaa is Asn, Leu, Gln, Gly, Phe, Val, Lys, Ser, Arg, Thr, His or Pro
XAXNLAX

SEQ ID NO: 200 - protein, 9 aa, synthetic construct
  REGION 1..9: note = synthetic
  SITE 2: note = MISC_FEATURE - Xaa is Leu or His
  SITE 4: note = MISC_FEATURE - Xaa is Trp, Phe, Tyr, Pro, Asn, Val, Lys, Met, Leu, Gly or Ser
QXFXGPPLT

SEQ ID NO: 201 - protein, 5 aa, synthetic construct
  REGION 1..5: note = synthetic
TSWMN

SEQ ID NO: 202 - protein, 17 aa, synthetic construct
  REGION 1..17: note = synthetic
RIYPGDADTNYVGKFKG

SEQ ID NO: 203 - protein, 15 aa, synthetic construct
  REGION 1..15: note = synthetic
SGTFNFGNYRWYFDV

SEQ ID NO: 204 - protein, 11 aa, synthetic construct
  REGION 1..11: note = synthetic
RVSENIYSHLA

SEQ ID NO: 205 - protein, 7 aa, synthetic construct
  REGION 1..7: note = synthetic
AATNLAL

SEQ ID NO: 206 - protein, 9 aa, synthetic construct
  REGION 1..9: note = synthetic
QLFWGPPLT

SEQ ID NO: 207 - protein, 118 aa, synthetic construct; identical to SEQ ID NO: 5
  REGION 1..118: note = synthetic

SEQ ID NO: 208 - protein, 447 aa, synthetic construct
  REGION 1..447: note = synthetic
QVQLVQSGAEVKKPGASVKVSCKASGYTLTDYTIHWVRQAPGQRLEWMGWIYPGSGNTKYNDKFKVRVTITRDTSASTAYMELSSLRSEDTAVYYCARRNYYAMEYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 209 - protein, 108 aa, synthetic construct; identical to SEQ ID NO: 17
  REGION 1..108: note = synthetic

SEQ ID NO: 210 - protein, 214 aa, synthetic construct
  REGION 1..214: note = synthetic
DIVMTQSPDSLAVSLGERATINCKASQNVFTNVAWYQQKPGQSPKPLIYSASYRYSGVPDRFSGSGSGTDFTLTISSLQAEDVAVYYCQQYSTYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 211 - protein, 5 aa, synthetic construct
  REGION 1..5: note = synthetic
DYTIH

SEQ ID NO: 212 - protein, 17 aa, synthetic construct
  REGION 1..17: note = synthetic
WIYPGSGNTKYNDKFKV

SEQ ID NO: 213 - protein, 8 aa, synthetic construct
  REGION 1..8: note = synthetic
RNYYAMEY

SEQ ID NO: 214 - protein, 11 aa, synthetic construct
  REGION 1..11: note = synthetic
KASQNVFTNVA

SEQ ID NO: 215 - protein, 7 aa, synthetic construct
  REGION 1..7: note = synthetic
SASYRYS

SEQ ID NO: 216 - protein, 9 aa, synthetic construct
  REGION 1..9: note = synthetic
QQYSTYPYT

SEQ ID NO: 217 - protein, 117 aa, synthetic construct
  REGION 1..117: note = synthetic
QVQLVQSGAEVKKPGASVKVSCKASGYTLTDYTIHWVRQAPGQRLEWMGWIYPGSGNTKYNDEFKVRVTITRDTSASTAYMELSSLRSEDTAVYYCARRNYYAMEYWGQGTTVTVSS

SEQ ID NO: 218 - protein, 444 aa, synthetic construct
  REGION 1..444: note = synthetic
QVQLVQSGAEVKKPGASVKVSCKASGYTLTDYTIHWVRQAPGQRLEWMGWIYPGSGNTKYNDEFKVRVTITRDTSASTAYMELSSLRSEDTAVYYCARRNYYAMEYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 219 - protein, 108 aa, synthetic construct
  REGION 1..108: note = synthetic
DIQMTQSPSSLSASVGDRVTITCKASQNVFTNVAWFQQKPGKAPKPLIYSASYRYSGVPSRFSGSGSGTDFTLTISSLQPEDFATYYCQQYSTYPYTFGQGTKLEIKR

SEQ ID NO: 220 - protein, 214 aa, synthetic construct
  REGION 1..214: note = synthetic
DIQMTQSPSSLSASVGDRVTITCKASQNVFTNVAWFQQKPGKAPKPLIYSASYRYSGVPSRFSGSGSGTDFTLTISSLQPEDFATYYCQQYSTYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 221 - protein, 5 aa, synthetic construct
  REGION 1..5: note = synthetic
DYTIH

SEQ ID NO: 222 - protein, 17 aa, synthetic construct
  REGION 1..17: note = synthetic
WIYPGSGNTKYNDEFKV

SEQ ID NO: 223 - protein, 8 aa, synthetic construct
  REGION 1..8: note = synthetic
RNYYAMEY

SEQ ID NO: 224 - protein, 11 aa, synthetic construct
  REGION 1..11: note = synthetic
KASQNVFTNVA

SEQ ID NO: 225 - protein, 7 aa, synthetic construct
  REGION 1..7: note = synthetic
SASYRYS

SEQ ID NO: 226 - protein, 9 aa, synthetic construct
  REGION 1..9: note = synthetic
QQYSTYPYT

SEQ ID NO: 227 - protein, 14 aa, synthetic construct
  REGION 1..14: note = synthetic
DPFELECPVKYCAN

SEQ ID NO: 228 - protein, 7 aa, synthetic construct
  REGION 1..7: note = synthetic
LHFEPVL

SEQ ID NO: 229 - protein, 20 aa, synthetic construct
  REGION 1..20: note = synthetic
HSILAGDPFELECPVKYCAN

SEQ ID NO: 230 - protein, 10 aa, synthetic construct
  REGION 1..10: note = synthetic
LEDRQTSWKE

SEQ ID NO: 231 - protein, 7 aa, synthetic construct
  REGION 1..7: note = synthetic
LHFEPVL

SEQ ID NO: 232 - skipped

SEQ ID NO: 233 - protein, 13 aa, synthetic construct
  REGION 1..13: note = synthetic
DPFELECPVKYCA

SEQ ID NO: 234 - protein, 8 aa, synthetic construct
  REGION 1..8: note = synthetic
IESHSTTL